SEQUENCE LISTING (328 sequences)

SEQ ID NO: 1 - DNA, 93 bp, artificial sequence
  synthetic transcription template
catcgatgctagtcgtaacgatccnnnnnnnnnnnnnnnnnnnnnnnnnnnnnncgagaacgttctctcctctccctatagtgagtcgtatta

SEQ ID NO: 2 - DNA, 92 bp, artificial sequence
  synthetic transcription template
catgcatcgcgactgactagccgnnnnnnnnnnnnnnnnnnnnnnnnnnnnnngtagaacgttctctcctctccctatagtgagtcgtatta

SEQ ID NO: 3 - DNA, 92 bp, artificial sequence
  synthetic transcription template
catcgatcgatcgatcgacagcgnnnnnnnnnnnnnnnnnnnnnnnnnnnnnngtagaacgttctctcctctccctatagtgagtcgtatta

SEQ ID NO: 4 - DNA, 76 bp, artificial sequence
  synthetic transcription template
gggagacaagaataaagcgagttnnnnnnnnnnnnnnnnnnnnnnnnnnnnnnaagagtcgatgatgcttagctag

SEQ ID NO: 5 - DNA, 50 bp, artificial sequence
  synthetic primer
gatcgatcgatcgatcgatctaatacgactcactatagggagacaagaat

SEQ ID NO: 6 - DNA, 23 bp, artificial sequence
  synthetic primer
ctagctaagcatcatcgactctt

SEQ ID NO: 7 - DNA, 77 bp, artificial sequence
  synthetic aptamer
gggagacaagaauaaagcgaguuuguauucuuucgaacuuucuaacacaucacaagagucgaugaugcuuagcuagt

SEQ ID NO: 8 - DNA, 77 bp, artificial sequence
  synthetic aptamer
gggagacaagaauaaagcgaguuugcuaugucauucuaguucacaucuucacaaagagucgaugaugcuuagcuagt

SEQ ID NO: 9 - RNA, 123 nt, artificial sequence
  synthetic aptamer
gggagacaagaauaaagcgaguuaagcaguagauugauauccgauagaaaaguaagagucgaugaugcuuagcuagaagggcgaauucgcggccgcuaaauucauuucgaccucucugcuagc

SEQ ID NO: 10 - RNA, 47 nt, artificial sequence
  synthetic aptamer
gggagacaagaauaaagcgaguuaacaauuucgaccucucugcuagc

SEQ ID NO: 11 - RNA, 57 nt, artificial sequence
  synthetic aptamer
gggagacaagaauaaagcgaguucaaacacagcauccguuucgaccucucugcuagc

SEQ ID NO: 12 - RNA, 54 nt, artificial sequence
  synthetic aptamer
gggagacaagaauaaagcgaguuacacagugccaguuucgaccucucugcuagc

SEQ ID NO: 13 - RNA, 43 nt, artificial sequence
  synthetic aptamer
gggagacaagaauaaagcgaguuuuuucgaccucucugcuagc

SEQ ID NO: 14 - RNA, 47 nt, artificial sequence
  synthetic aptamer
gggagacaagaauaaagcgaguuuguuguuucgaccucucugcuagc

SEQ ID NO: 15 - RNA, 52 nt, artificial sequence
  synthetic aptamer
gggagacaagaauaaagcgaguuuggaguuagauuucgaccucucugcuagc

SEQ ID NO: 16 - RNA, 122 nt, artificial sequence
  synthetic aptamer
gggagacaagaauaaagcgaguuccacacuaaauggaggguuggaauuagucaagagucgaugaugcuuagcuagaagggcgaauucgcggccgcuaaauucauuucgaccucucugcuagc

SEQ ID NO: 17 - RNA, 50 nt, artificial sequence
  synthetic aptamer
gggagacaagaauaaagcgaguucgaucuucuuucgaccucucugcuagc

SEQ ID NO: 18 - RNA, 47 nt, artificial sequence
  synthetic aptamer
gggagacaagaauaaagcgaguuugccuuuucgaccucucugcuagc

SEQ ID NO: 19 - RNA, 54 nt, artificial sequence
  synthetic aptamer
gggagacaagaauaaagcgaguuuaaaauagugauuuucgaccucucugcuagc

SEQ ID NO: 20 - RNA, 52 nt, artificial sequence
  synthetic aptamer
gggagacaagaauaaagcgaguugacaaucacuuuucgaccucucugcuagc

SEQ ID NO: 21 - RNA, 48 nt, artificial sequence
  synthetic aptamer
gggagacaagaauaaagcgaguuaguccauuucgaccucucugcuagc

SEQ ID NO: 22 - RNA, 123 nt, artificial sequence
  synthetic aptamer
gggagacaagaauaaagcgaguuccguagugcaaggcucaaggucuaccacugaagagucgaugaugcuuagcuagaagggcgaauucgcggccgcuaaauucauuucgaccucucugcuagc

SEQ ID NO: 23 - RNA, 50 nt, artificial sequence
  synthetic aptamer
gggagacaagaauaaagcgaguuugcagaucuuucgaccucucugcuagc

SEQ ID NO: 24 - RNA, 119 nt, artificial sequence
  synthetic aptamer
gggagacaagaauaaagcgaguuucguaacuaauguagaugguggauaaguucaagagucgaugaugcuuagcuagaagggcgaauucgcggccgcuaaauuucgaccucucugcuagc

SEQ ID NO: 25 - RNA, 46 nt, artificial sequence
  synthetic aptamer
gggagacaagaauaaagcgaguuacguuuucgaccucucugcuagc

SEQ ID NO: 26 - RNA, 77 nt, artificial sequence
  synthetic aptamer
gggagacaagaauaaagcgaguuaucagccuauccguuaaagauuauucauugaagaguuucgaccucucugcuagc

SEQ ID NO: 27 - RNA, 45 nt, artificial sequence
  synthetic aptamer
gggagacaagaauaaagcgaguucaguuucgaccucucugcuagc

SEQ ID NO: 28 - RNA, 49 nt, artificial sequence
  synthetic aptamer
gggagacaagaauaaagcgaguucuguacguuucgaccucucugcuagc

SEQ ID NO: 29 - RNA, 50 nt, artificial sequence
  synthetic aptamer
gggagacaagaauaaagcgaguuacggagauuuucgaccucucugcuagc

SEQ ID NO: 30 - RNA, 123 nt, artificial sequence
  synthetic aptamer
gggagacaagaauaaagcgaguuccuaaaauucaccgagauuuaucaggcguuaagagucgaugaugcuuagcuagaagggcgaauucgcggccgcuaaauucauuucgaccucucugcuagc

SEQ ID NO: 31 - RNA, 55 nt, artificial sequence
  synthetic aptamer
gggagacaagaauaaagcgaguuuuuuaaagauguguuucgaccucucugcuagc

SEQ ID NO: 32 - RNA, 57 nt, artificial sequence
  synthetic aptamer
gggagacaagaauaaagcgaguuuaagguuuacucccauuucgaccucucugcuagc

SEQ ID NO: 33 - RNA, 49 nt, artificial sequence
  synthetic aptamer
gggagacaagaauaaagcgaguuugaacccuuucgaccucucugcuagc

SEQ ID NO: 34 - RNA, 54 nt, artificial sequence
  synthetic aptamer
gggagacaagaauaaagcgaguucacuccguuuaauuucgaccucucugcuagc

SEQ ID NO: 35 - RNA, 45 nt, artificial sequence
  synthetic aptamer
gggagacaagaauaaagcgaguuauuuuucgaccucucugcuagc

SEQ ID NO: 36 - RNA, 43 nt, artificial sequence
  synthetic aptamer
gggagacaagaauaaagcgaguuauuucgaccucucugcuagc

SEQ ID NO: 37 - RNA, 123 nt, artificial sequence
  synthetic aptamer
gggagacaagaauaaagcgaguuucucuggccucgauuuuguuaccauagcuaaagagucgaugaugcuuagcuagaagggcgaauucgcggccgcuaaauucauuucgaccucucugcuagc

SEQ ID NO: 38 - RNA, 123 nt, artificial sequence
  synthetic aptamer
gggagacaagaauaaagcgaguugacagcuuuauuucaugcaaaacaaaacggaagagucgaugaugcuuagcuagaagggcgaauucgcggccgcuaaauucauuucgaccucucugcuagc

SEQ ID NO: 39 - RNA, 52 nt, artificial sequence
  synthetic aptamer
gggagacaagaauaaagcgaguuaauucuacucuuucgaccucucugcuagc

SEQ ID NO: 40 - RNA, 123 nt, artificial sequence
  synthetic aptamer
gggagacaagaauaaagcgaguuacacauaccgucaucgcacggucuuaagcaaagagucgaugaugcuuagcuagaagggcgaauucgcggccgcuaaauucauuucgaccucucugcuagc

SEQ ID NO: 41 - RNA, 146 nt, artificial sequence
  synthetic aptamer
gggagacaagaauaaagcgaguuauagcacuggcguacuguucauuaauuagcaagagucgaugaugcuuagcuagaagggcgaauucguuuaaaccugcaggacuagucccuuuagugaggguuaauuucgaccucucugcuagc

SEQ ID NO: 42 - RNA, 53 nt, artificial sequence
  synthetic aptamer
gggagacaagaauaaagcgaguugauaauuaccauuucgaccucucugcuagc

SEQ ID NO: 43 - RNA, 123 nt, artificial sequence
  synthetic aptamer
gggagacaagaauaaagcgaguuccaaaugaguuguuuccuuaaaaaaacaugaagagucgaugaugcuuagcuagaagggcgaauucgcggccgcuaaauucauuucgaccucucugcuagc

SEQ ID NO: 44 - RNA, 49 nt, artificial sequence
  synthetic aptamer
gggagacaagaauaaagcgaguucaaagcauuucgaccucucugcuagc

SEQ ID NO: 45 - RNA, 123 nt, artificial sequence
  synthetic aptamer
gggagacaagaauaaagcgaguuguaccaaaaugguuguaaaccauaucacgcaagagucgaugaugcuuagcuagaagggcgaauucgcggccgcuaaauucauuucgaccucucugcuagc

SEQ ID NO: 46 - RNA, 48 nt, artificial sequence
  synthetic aptamer
gggagacaagaauaaagcgaguuauguaauuucgaccucucugcuagc

SEQ ID NO: 47 - RNA, 119 nt, artificial sequence
  synthetic aptamer
gggagacaagaauaaagcgaguuguauaucuuaacgcaagcgagguaauuugcaagagucgaugaugcuuagcuagaagggcgaauucgcggccgcuaaauuucgaccucucugcuagc

SEQ ID NO: 48 - RNA, 56 nt, artificial sequence
  synthetic aptamer
gggagacaagaauaaagcgaguuauuguugcauacgcuuucgaccucucugcuagc

SEQ ID NO: 49 - RNA, 76 nt, artificial sequence
  synthetic aptamer
gggagacaagaauaaagcgaguuuaccuucaugaaugcaaacagcaugacauaaagagucgaugaugcuuagcuag

SEQ ID NO: 50 - RNA, 76 nt, artificial sequence
  synthetic aptamer
gggagacaagaauaaagcgaguuuucacucucugccauuuucaaaaugacacgaagagucgaugaugcuuagcuag

SEQ ID NO: 51 - RNA, 76 nt, artificial sequence
  synthetic aptamer
gggagacaagaauaaagcgaguuguauuuuugcgcacuagauaacaucucacaaagagucgaugaugcuuagcuag

SEQ ID NO: 52 - RNA, 76 nt, artificial sequence
  synthetic aptamer
gggagacaagaauaaagcgaguuugugcaacaaaagcuugcgaccuguauaucaagagucgaugaugcuuagcuag

SEQ ID NO: 53 - RNA, 76 nt, artificial sequence
  synthetic aptamer
gggagacaagaauaaagcgaguuuuucuagcgucacacucagaauagaauucaaagagucgaugaugcuuagcuag

SEQ ID NO: 54 - RNA, 76 nt, artificial sequence
  synthetic aptamer
gggagacaagaauaaagcgaguuagcacauuacuuuucuugcaauagcagugaaagagucgaugaugcuuagcuag

SEQ ID NO: 55 - RNA, 76 nt, artificial sequence
  synthetic aptamer
gggagacaagaauaaagcgaguuuguuauaucacgucacuuaguuagcaucacaagagucgaugaugcuuagcuag

SEQ ID NO: 56 - RNA, 76 nt, artificial sequence
  synthetic aptamer
gggagacaagaauaaagcgaguugucauggcaguaucacaacucuaguuaacuaagagucgaugaugcuuagcuag

SEQ ID NO: 57 - RNA, 76 nt, artificial sequence
  synthetic aptamer
gggagacaagaauaaagcgaguucagcauacuguugugcacaucaaccauccaaagagucgaugaugcuuagcuag

SEQ ID NO: 58 - RNA, 76 nt, artificial sequence
  synthetic aptamer
gggagacaagaauaaagcgaguuagcucaugcccuuuaucguauugacaauauaagagucgaugaugcuuagcuag

SEQ ID NO: 59 - RNA, 76 nt, artificial sequence
  synthetic aptamer
gggagacaagaauaaagcgaguuuccacaaaucaaucaaccuaacguccucacaagagucgaugaugcuuagcuag

SEQ ID NO: 60 - RNA, 76 nt, artificial sequence
  synthetic aptamer
gggagacaagaauaaagcgaguuucaaacuacgcagauugcuaucacaucaccaagagucgaugaugcuuagcuag

SEQ ID NO: 61 - RNA, 76 nt, artificial sequence
  synthetic aptamer
gggagacaagaauaaagcgaguucucaacacucauguaauagucagcuucacuaagagucgaugaugcuuagcuag

SEQ ID NO: 62 - RNA, 76 nt, artificial sequence
  synthetic aptamer
gggagacaagaauaaagcgaguuuccuugaaccaaaauaucuaucauaucacaaagagucgaugaugcuuagcuag

SEQ ID NO: 63 - RNA, 75 nt, artificial sequence
  synthetic aptamer
gggagacaagaauaaagcgaguuggcauucgcauuaaucaugauuaacuacgaagagucgaugaugcuuagcuag

SEQ ID NO: 64 - RNA, 76 nt, artificial sequence
  synthetic aptamer
gggagacaagaauaaagcgaguugccagcuugccugcuuccaucauuacauguaagagucgaugaugcuuagcuag

SEQ ID NO: 65 - RNA, 76 nt, artificial sequence
  synthetic aptamer
gggagacaagaauaaagcgaguucauggcauuagguugugaguccacaaauuuaagagucgaugaugcuuagcuag

SEQ ID NO: 66 - RNA, 76 nt, artificial sequence
  synthetic aptamer
gggagacaagaauaaagcgaguuaucccaugcacaucuaguuuucauuaucacaagagucgaugaugcuuagcuag

SEQ ID NO: 67 - RNA, 76 nt, artificial sequence
  synthetic aptamer
gggagacaagaauaaagcgaguucuugacauucaguugccuuagacacacaugaagagucgaugaugcuuagcuag

SEQ ID NO: 68 - RNA, 76 nt, artificial sequence
  synthetic aptamer
gggagacaagaauaaagcgaguugcaaaaacacgcgcaaguuaacuuccucacaagagucgaugaugcuuagcuag

SEQ ID NO: 69 - RNA, 76 nt, artificial sequence
  synthetic aptamer
gggagacaagaauaaagcgaguuggcauuacuucaacugcaagcguuguuaguaagagucgaugaugcuuagcuag

SEQ ID NO: 70 - RNA, 76 nt, artificial sequence
  synthetic aptamer
gggagacaagaauaaagcgaguucucauucuuaguuacuguuucgcgugacauaagagucgaugaugcuuagcuag

SEQ ID NO: 71 - RNA, 76 nt, artificial sequence
  synthetic aptamer
gggagacaagaauaaagcgaguuggacuuacuacagcuaucauaaucacggucaagagucgaugaugcuuagcuag

SEQ ID NO: 72 - RNA, 76 nt, artificial sequence
  synthetic aptamer
gggagacaagaauaaagcgaguuacaacucauaguuaucaucaugcucacuucaagagucgaugaugcuuagcuag

SEQ ID NO: 73 - RNA, 76 nt, artificial sequence
  synthetic aptamer
gggagacaagaauaaagcgaguuguacuuccaauaauccagcucucauaucacaagagucgaugaugcuuagcuag

SEQ ID NO: 74 - RNA, 77 nt, artificial sequence
  synthetic aptamer
gggagacaagaauaaagcgaguuugacagcuagaccucgcucuugcgcuaagucaagagucgaugaugcuuagcuag

SEQ ID NO: 75 - RNA, 75 nt, artificial sequence
  synthetic aptamer
gggagacaagaauaaagcgaguucacgcacuuugacauuuucacuauggcacaagagucgaugaugcuuagcuag

SEQ ID NO: 76 - RNA, 76 nt, artificial sequence
  synthetic aptamer
gggagacaagaauaaagcgaguucaagauucucauaucucacuuaggcuuuugaagagucgaugaugcuuagcuag

SEQ ID NO: 77 - RNA, 76 nt, artificial sequence
  synthetic aptamer
gggagacaagaauaaagcgaguuuuccucacuuaaaaaugcuaccacugucacaagagucgaugaugcuuagcuag

SEQ ID NO: 78 - RNA, 76 nt, artificial sequence
  synthetic aptamer
gggagacaagaauaaagcgaguuuauuaacaucaccacacuuaggacaaacguaagagucgaugaugcuuagcuag

SEQ ID NO: 79 - RNA, 76 nt, artificial sequence
  synthetic aptamer
gggagacaagaauaaagcgaguuuagcaaaaucuguuaugcuaaucgaucuguaagagucgaugaugcuuagcuag

SEQ ID NO: 80 - RNA, 76 nt, artificial sequence
  synthetic aptamer
gggagacaagaauaaagcgaguucugccuuacuuacaauagcaagaucagcccaagagucgaugaugcuuagcuag

SEQ ID NO: 81 - RNA, 76 nt, artificial sequence
  synthetic aptamer
gggagacaagaauaaagcgaguuugaucuuaguagcuuuaauuacgcggacauaagagucgaugaugcuuagcuag

SEQ ID NO: 82 - RNA, 75 nt, artificial sequence
  synthetic aptamer
gggagacaagaauaaagcgaguucauggcauuacuuguacacuucgcaaaguaagagucgaugaugcuuagcuag

SEQ ID NO: 83 - RNA, 76 nt, artificial sequence
  synthetic aptamer
gggagacaagaauaaagcgaguuuccacaauucaaucucuaauaacuuaucacaagagucgaugaugcuuagcuag

SEQ ID NO: 84 - RNA, 76 nt, artificial sequence
  synthetic aptamer
gggagacaagaauaaagcgaguuuccacaaacaaaauuuacuaacacuaucacaagagucgaugaugcuuagcuag

SEQ ID NO: 85 - RNA, 76 nt, artificial sequence
  synthetic aptamer
gggagacaagaauaaagcgaguucggguacuaaacacaacgaccuaucccaaaaagagucgaugaugcuuagcuag

SEQ ID NO: 86 - RNA, 76 nt, artificial sequence
  synthetic aptamer
gggagacaagaauaaagcgaguuauauaugucaauccaagcuaacucaaucacaagagucgaugaugcuuagcuag

SEQ ID NO: 87 - RNA, 76 nt, artificial sequence
  synthetic aptamer
gggagacaagaauaaagcgaguugcuagcgucaugucauuugauaucaaucacaagagucgaugaugcuuagcuag

SEQ ID NO: 88 - RNA, 76 nt, artificial sequence
  synthetic aptamer
gggagacaagaauaaagcgaguuuaccuacuaucucaaagcucucaccaucacaagagucgaugaugcuuagcuag

SEQ ID NO: 89 - RNA, 76 nt, artificial sequence
  synthetic aptamer
gggagacaagaauaaagcgaguucaaacagcaagcuaacacgucaccauuuucaagagucgaugaugcuuagcuag

SEQ ID NO: 90 - RNA, 76 nt, artificial sequence
  synthetic aptamer
gggagacaagaauaaagcgaguucgaugaucauugcuaauucgugcuaguaucaagagucgaugaugcuuagcuag

SEQ ID NO: 91 - RNA, 76 nt, artificial sequence
  synthetic aptamer
gggagacaagaauaaagcgaguuucaucauccaauaccuaucuaacuaaucacaagagucgaugaugcuuagcuag

SEQ ID NO: 92 - RNA, 76 nt, artificial sequence
  synthetic aptamer
gggagacaagaauaaagcgaguucucauucuuucaacgcacauggaugcuguaaagagucgaugaugcuuagcuag

SEQ ID NO: 93 - RNA, 76 nt, artificial sequence
  synthetic aptamer
gggagacaagaauaaagcgaguuaauucacuacuaugcacaucugccaauuucaagagucgaugaugcuuagcuag

SEQ ID NO: 94 - RNA, 76 nt, artificial sequence
  synthetic aptamer
gggagacaagaauaaagcgaguuagucguuucaagcucacgccucuacacaguaagagucgaugaugcuuagcuag

SEQ ID NO: 95 - RNA, 76 nt, artificial sequence
  synthetic aptamer
gggagacaagaauaaagcgaguuguaaacgugaaauuagcuagcaccacuacuaagagucgaugaugcuuagcuag

SEQ ID NO: 96 - RNA, 76 nt, artificial sequence
  synthetic aptamer
gggagacaagaauaaagcgaguucauucuuggcauucuaacuuucacugucacaagagucgaugaugcuuagcuag

SEQ ID NO: 97 - RNA, 76 nt, artificial sequence
  synthetic aptamer
gggagacaagaauaaagcgaguuacuagcacaucaccuuuccucauauaggcaaagagucgaugaugcuuagcuag

SEQ ID NO: 98 - RNA, 76 nt, artificial sequence
  synthetic aptamer
gggagacaagaauaaagcgaguuuccgcaaauccaaacuaccucacacaucacaagagucgaugaugcuuagcuag

SEQ ID NO: 99 - RNA, 75 nt, artificial sequence
  synthetic aptamer
gggagacaagaauaaagcgaguuggccuuuaucggcgaucaaacaccagauaaagagucgaugaugcuuagcuag

SEQ ID NO: 100 - RNA, 76 nt, artificial sequence
  synthetic aptamer
gggagacaagaauaaagcgaguuuguauucuuucgaacuuucuaacacaucacaagagucgaugaugcuuagcuag

SEQ ID NO: 101 - RNA, 76 nt, artificial sequence
  synthetic aptamer
gggagacaagaauaaagcgaguucgcucauugucaaucuagcuuccacgucacaagagucgaugaugcuuagcuag

SEQ ID NO: 102 - RNA, 76 nt, artificial sequence
  synthetic aptamer
gggagacaagaauaaagcgaguuuccacgcaccaaucuaacuaacacuaucacaagagucgaugaugcuuagcuag

SEQ ID NO: 103 - RNA, 75 nt, artificial sequence
  synthetic aptamer
gggagacaagaauaaagcgaguuugcuuugucaaucuaacuaacacaaucacaagagucgaugaugcuuagcuag

SEQ ID NO: 104 - RNA, 76 nt, artificial sequence
  synthetic aptamer
gggagacaagaauaaagcgaguuugcuaugucauucuaguucacaucuucacaaagagucgaugaugcuuagcuag

SEQ ID NO: 105 - RNA, 76 nt, artificial sequence
  synthetic aptamer
gggagacaagaauaaagcgaguuuccacacacagucucuaacuaacuauucacaagagucgaugaugcuuagcuag

SEQ ID NO: 106 - RNA, 74 nt, artificial sequence
  synthetic aptamer
gggagacaagaauaaagcgaguuuauucuuucgaacuuucuaacacaucacaagagucgaugaugcuuagcuag

SEQ ID NO: 107 - RNA, 76 nt, artificial sequence
  synthetic aptamer
gggagacaagaauaaagcgaguuccacacauccaaacuaguuaacaccaacacaagagucgaugaugcuuagcuag

SEQ ID NO: 108 - RNA, 76 nt, artificial sequence
  synthetic aptamer
gggagacaagaauaaagcgaguuggcuuaugucaaacuagauaacauccuuacaagagucgaugaugcuuagcuag

SEQ ID NO: 109 - RNA, 76 nt, artificial sequence
  synthetic aptamer
gggagacaagaauaaagcgaguucguuuugucaaacuagcuaacuacaacacaaagagucgaugaugcuuagcuag

SEQ ID NO: 110 - RNA, 76 nt, artificial sequence
  synthetic aptamer
gggagacaagaauaaagcgaguuacgccaugucauacuuucuaacacaucacaaagagucgaugaugcuuagcuag

SEQ ID NO: 111 - RNA, 76 nt, artificial sequence
  synthetic aptamer
gggagacaagaauaaagcgaguuuccuccaagucuuacuaacuuucacaucacaagagucgaugaugcuuagcuag

SEQ ID NO: 112 - RNA, 76 nt, artificial sequence
  synthetic aptamer
gggagacaagaauaaagcgaguuuccacaaauaaucacuuguuaacacaucacaagagucgaugaugcuuagcuag

SEQ ID NO: 113 - RNA, 76 nt, artificial sequence
  synthetic aptamer
gggagacaagaauaaagcgaguuuccacacauccaaacuagucaacuuaucacaagagucgaugaugcuuagcuag

SEQ ID NO: 114 - RNA, 76 nt, artificial sequence
  synthetic aptamer
gggagacaagaauaaagcgaguuuuucuccacacacaccaguuaacuuaucacaagagucgaugaugcuuagcuag

SEQ ID NO: 115 - RNA, 75 nt, artificial sequence
  synthetic aptamer
gggagacaagaauaaagcgaguuccacaccauccaacuaacuaacuuaucacaagagucgaugaugcuuagcuag

SEQ ID NO: 116 - RNA, 76 nt, artificial sequence
  synthetic aptamer
gggagacaagaauaaagcgaguugugcuucuaucaaucuaguuaacuuaucacaagagucgaugaugcuuagcuag

SEQ ID NO: 117 - RNA, 76 nt, artificial sequence
  synthetic aptamer
gggagacaagaauaaagcgaguucuauucuuugcaacuagcugacacuaucacaagagucgaugaugcuuagcuag

SEQ ID NO: 118 - RNA, 76 nt, artificial sequence
  synthetic aptamer
gggagacaagaauaaagcgaguuauugcauguuaguccucguagacucuccauaagagucgaugaugcuuagcuag

SEQ ID NO: 119 - RNA, 76 nt, artificial sequence
  synthetic aptamer
gggagacaagaauaaagcgaguuuccacgauccacaacuaucuaacauaucacaagagucgaugaugcuuagcuag

SEQ ID NO: 120 - RNA, 76 nt, artificial sequence
  synthetic aptamer
gggagacaagaauaaagcgaguuuccgcgaauaacaucuaacuaacuaaucacaagagucgaugaugcuuagcuag

SEQ ID NO: 121 - RNA, 76 nt, artificial sequence
  synthetic aptamer
gggagacaagaauaaagcgaguucacgaucuacaccgauuaucuuauugguguaagagucgaugaugcuuagcuag

SEQ ID NO: 122 - RNA, 77 nt, artificial sequence
  synthetic aptamer
gggagacaagaauaaagcgaguuugucaaacggcgucacacucuucuauaauguaagagucgaugaugcuuagcuag

SEQ ID NO: 123 - RNA, 76 nt, artificial sequence
  synthetic aptamer
gggagacaagaauaaagcgaguuugcuaugucuauacuagcuuccauugucacaagagucgaugaugcuuagcuag

SEQ ID NO: 124 - RNA, 76 nt, artificial sequence
  synthetic aptamer
gggagacaagaauaaagcgaguuuccacaaacgcaaucuagcuaucauaucacaagagucgaugaugcuuagcuag

SEQ ID NO: 125 - RNA, 76 nt, artificial sequence
  synthetic aptamer
gggagacaagaauaaagcgaguuacgcucaugucaucuagcuuccaacgucacaagagucgaugaugcuuagcuag

SEQ ID NO: 126 - RNA, 73 nt, artificial sequence
  synthetic aptamer
gggagacaagaauaaagcgaguuguuugucaaucuagcuuccaauaucacaagagucgaugaugcuuagcuag

SEQ ID NO: 127 - RNA, 76 nt, artificial sequence
  synthetic aptamer
gggagacaagaauaaagcgaguuuugucaucuaucgucacgccucuacacaguaagagucgaugaugcuuagcuag

SEQ ID NO: 128 - RNA, 76 nt, artificial sequence
  synthetic aptamer
gggagacaagaauaaagcgaguuuccgcgaaacacaucuagcucacauuucacaagagucgaugaugcuuagcuag

SEQ ID NO: 129 - RNA, 76 nt, artificial sequence
  synthetic aptamer
gggagacaagaauaaagcgaguuuacgcauugucauccuaguuaacauaucacaagagucgaugaugcuuagcuag

SEQ ID NO: 130 - RNA, 76 nt, artificial sequence
  synthetic aptamer
gggagacaagaauaaagcgaguuguauucuuuucacacuaucuuccaugacacaagagucgaugaugcuuagcuag

SEQ ID NO: 131 - RNA, 76 nt, artificial sequence
  synthetic aptamer
gggagacaagaauaaagcgaguuucugcaaccacaaacuaauuaacacaucacaagagucgaugaugcuuagcuag

SEQ ID NO: 132 - RNA, 76 nt, artificial sequence
  synthetic aptamer
gggagacaagaauaaagcgaguuguguucuugcaaacuaacuaacgugaucacaagagucgaugaugcuuagcuag

SEQ ID NO: 133 - RNA, 76 nt, artificial sequence
  synthetic aptamer
gggagacaagaauaaagcgaguuguauucuugcaaacuaucuaucauuguuacaagagucgaugaugcuuagcuag

SEQ ID NO: 134 - RNA, 76 nt, artificial sequence
  synthetic aptamer
gggagacaagaauaaagcgaguuauauucuuaucaacuaguugacgccaacacaagagucgaugaugcuuagcuag

SEQ ID NO: 135 - RNA, 76 nt, artificial sequence
  synthetic aptamer
gggagacaagaauaaagcgaguuacgcguugucaaucuaguuaacauaaucacaagagucgaugaugcuuagcuag

SEQ ID NO: 136 - RNA, 76 nt, artificial sequence
  synthetic aptamer
gggagacaagaauaaagcgaguuauccgcaccaaccaucuacuaucauaucacaagagucgaugaugcuuagcuag

SEQ ID NO: 137 - RNA, 76 nt, artificial sequence
  synthetic aptamer
gggagacaagaauaaagcgaguuugccauugucaccuaguuaacaucaacacaaagagucgaugaugcuuagcuag

SEQ ID NO: 138 - RNA, 76 nt, artificial sequence
  synthetic aptamer
gggagacaagaauaaagcgaguuugcuuuugucacacuauuuaucccaucacaaagagucgaugaugcuuagcuag

SEQ ID NO: 139 - RNA, 76 nt, artificial sequence
  synthetic aptamer
gggagacaagaauaaagcgaguuuccacgaaccaaucuagcuuccaacuucacaagagucgaugaugcuuagcuag

SEQ ID NO: 140 - RNA, 76 nt, artificial sequence
  synthetic aptamer
gggagacaagaauaaagcgaguuuccgcaaaaucaaucuaacuaacauaucacaagagucgaugaugcuuagcuag

SEQ ID NO: 141 - RNA, 76 nt, artificial sequence
  synthetic aptamer
gggagacaagaauaaagcgaguuauauuaaugucggccuagguaacacaucacaagagucgaugaugcuuagcuag

SEQ ID NO: 142 - RNA, 76 nt, artificial sequence
  synthetic aptamer
gggagacaagaauaaagcgaguuucagcacaaacaaacuagcucacauuucacaagagucgaugaugcuuagcuag

SEQ ID NO: 143 - RNA, 76 nt, artificial sequence
  synthetic aptamer
gggagacaagaauaaagcgaguugugcuaccaucaagcuagcuaacuuaucacaagagucgaugaugcuuagcuag

SEQ ID NO: 144 - RNA, 76 nt, artificial sequence
  synthetic aptamer
gggagacaagaauaaagcgaguuguauucuuaucaugcuuucuaacacaucacaagagucgaugaugcuuagcuag

SEQ ID NO: 145 - RNA, 76 nt, artificial sequence
  synthetic aptamer
gggagacaagaauaaagcgaguuucuucacaacauaacuaguuuccaugucacaagagucgaugaugcuuagcuag

SEQ ID NO: 146 - RNA, 76 nt, artificial sequence
  synthetic aptamer
gggagacaagaauaaagcgaguuuccucaaauacagcuagcuuccaccaccacaagagucgaugaugcuuagcuag

SEQ ID NO: 147 - RNA, 75 nt, artificial sequence
  synthetic aptamer
gggagacaagaauaaagcgaguuagcuaugucaaacuagcucuccuuaucacaagagucgaugaugcuuagcuag

SEQ ID NO: 148 - RNA, 76 nt, artificial sequence
  synthetic aptamer
gggagacaagaauaaagcgaguuguucucacaaaccacuagcucucauaucacaagagucgaugaugcuuagcuag

SEQ ID NO: 149 - RNA, 76 nt, artificial sequence
  synthetic aptamer
gggagacaagaauaaagcgaguuauauugcugucaaacuauuuaacuuaucacaagagucgaugaugcuuagcuag

SEQ ID NO: 150 - RNA, 76 nt, artificial sequence
  synthetic aptamer
gggagacaagaauaaagcgaguuauauucuuugcaacuugcuaacacuaucacaagagucgaugaugcuuagcuag

SEQ ID NO: 151 - RNA, 76 nt, artificial sequence
  synthetic aptamer
gggagacaagaauaaagcgaguuguaaucuugucaacuuaguuaacuuaucacaagagucgaugaugcuuagcuag

SEQ ID NO: 152 - RNA, 76 nt, artificial sequence
  synthetic aptamer
gggagacaagaauaaagcgaguugaucauccaaacacuaucuuccauccucacaagagucgaugaugcuuagcuag

SEQ ID NO: 153 - RNA, 76 nt, artificial sequence
  synthetic aptamer
gggagacaagaauaaagcgaguucacuaccuguaugugucaauagauccaaucaagagucgaugaugcuuagcuag

SEQ ID NO: 154 - RNA, 76 nt, artificial sequence
  synthetic aptamer
gggagacaagaauaaagcgaguuggaccuucucguuuaaaucagguuagcgucaagagucgaugaugcuuagcuag

SEQ ID NO: 155 - RNA, 76 nt, artificial sequence
  synthetic aptamer
gggagacaagaauaaagcgaguucccacaaaugccuucuagcuaacuuaucacaagagucgaugaugcuuagcuag

SEQ ID NO: 156 - RNA, 76 nt, artificial sequence
  synthetic aptamer
gggagacaagaauaaagcgaguuggccuccuaauauacgccuaacaacgucacaagagucgaugaugcuuagcuag

SEQ ID NO: 157 - RNA, 76 nt, artificial sequence
  synthetic aptamer
gggagacaagaauaaagcgaguuguauucuuaucgcacuagcucacugaucacaagagucgaugaugcuuagcuag

SEQ ID NO: 158 - RNA, 76 nt, artificial sequence
  synthetic aptamer
gggagacaagaauaaagcgaguuguucuuccuaacacuaguuuccauuuucacaagagucgaugaugcuuagcuag

SEQ ID NO: 159 - RNA, 76 nt, artificial sequence
  synthetic aptamer
gggagacaagaauaaagcgaguuagcuuugucacaacuauuuaacuuugucacaagagucgaugaugcuuagcuag

SEQ ID NO: 160 - RNA, 76 nt, artificial sequence
  synthetic aptamer
gggagacaagaauaaagcgaguugucauaaagcuuuugugaucgcucauagucaagagucgaugaugcuuagcuag

SEQ ID NO: 161 - RNA, 76 nt, artificial sequence
  synthetic aptamer
gggagacaagaauaaagcgaguucucuucacauacaagcaguuaacacaucacaagagucgaugaugcuuagcuag

SEQ ID NO: 162 - RNA, 76 nt, artificial sequence
  synthetic aptamer
gggagacaagaauaaagcgaguuuccacaacaaccuucuaacuaacuuaucacaagagucgaugaugcuuagcuag

SEQ ID NO: 163 - RNA, 76 nt, artificial sequence
  synthetic aptamer
gggagacaagaauaaagcgaguuuccacgucaaccaucuauauaacauaucacaagagucgaugaugcuuagcuag

SEQ ID NO: 164 - RNA, 76 nt, artificial sequence; identical to SEQ ID NO: 100
  synthetic aptamer

SEQ ID NO: 165 - RNA, 76 nt, artificial sequence
  synthetic aptamer
gggagacaagaauaaagcgaguugaucuaacaaguauuaguauaugcuauggcaagagucgaugaugcuuagcuag

SEQ ID NO: 166 - RNA, 76 nt, artificial sequence
  synthetic aptamer
gggagacaagaauaaagcgaguuauauucuuaucaacauaguuaacuuaucacaagagucgaugaugcuuagcuag

SEQ ID NO: 167 - RNA, 76 nt, artificial sequence
  synthetic aptamer
gggagacaagaauaaagcgaguuggccuuacauuguacuauaauucauaugucaagagucgaugaugcuuagcuag

SEQ ID NO: 168 - RNA, 76 nt, artificial sequence
  synthetic aptamer
gggagacaagaauaaagcgaguuugcuaugucaaucuagcuuccaucaacacaaagagucgaugaugcuuagcuag

SEQ ID NO: 169 - RNA, 76 nt, artificial sequence; identical to SEQ ID NO: 104
  synthetic aptamer

SEQ ID NO: 170 - RNA, 76 nt, artificial sequence
  synthetic aptamer
gggagacaagaauaaagcgaguuuguauucuuucggucuugcuaacuuaucacaagagucgaugaugcuuagcuag

SEQ ID NO: 171 - RNA, 76 nt, artificial sequence
  synthetic aptamer
gggagacaagaauaaagcgaguugacgcuaugucaucuagcuaucaacgucacaagagucgaugaugcuuagcuag

SEQ ID NO: 172 - RNA, 76 nt, artificial sequence
  synthetic aptamer
gggagacaagaauaaagcgaguuuccacacacagcaacuagcucacacaucacaagagucgaugaugcuuagcuag

SEQ ID NO: 173 - RNA, 76 nt, artificial sequence
  synthetic aptamer
gggagacaagaauaaagcgaguuuccacaaaucaaaccuaucuaacccaucacaagagucgaugaugcuuagcuag

SEQ ID NO: 174 - RNA, 76 nt, artificial sequence
  synthetic aptamer
gggagacaagaauaaagcgaguuucaucaaaaucacacuagccgacacaucacaagagucgaugaugcuuagcuag

SEQ ID NO: 175 - RNA, 76 nt, artificial sequence
  synthetic aptamer
gggagacaagaauaaagcgaguuugcuuuaucaaucuagcucacaucucaccuaagagucgaugaugcuuagcuag

SEQ ID NO: 176 - RNA, 76 nt, artificial sequence
  synthetic aptamer
gggagacaagaauaaagcgaguuuccacaaauacaaccuaucuaccuuaucacaagagucgaugaugcuuagcuag

SEQ ID NO: 177 - RNA, 76 nt, artificial sequence
  synthetic aptamer
gggagacaagaauaaagcgaguuucagcaaaccaaccauagcuuucaagucacaagagucgaugaugcuuagcuag

SEQ ID NO: 178 - RNA, 76 nt, artificial sequence
  synthetic aptamer
gggagacaagaauaaagcgaguucagcugucuuaauaaucuaucucguacacgaagagucgaugaugcuuagcuag

SEQ ID NO: 179 - RNA, 76 nt, artificial sequence
  synthetic aptamer
gggagacaagaauaaagcgaguuuccacaaaacaucccuaacuaacuagucacaagagucgaugaugcuuagcuag

SEQ ID NO: 180 - RNA, 76 nt, artificial sequence
  synthetic aptamer
gggagacaagaauaaagcgaguuaacuguaggcucacgaucuuauucaucaguaagagucgaugaugcuuagcuag

SEQ ID NO: 181 - RNA, 76 nt, artificial sequence
  synthetic aptamer
gggagacaagaauaaagcgaguuuuccacaaaucuuccuaguuaacuuaucacaagagucgaugaugcuuagcuag

SEQ ID NO: 182 - RNA, 76 nt, artificial sequence
  synthetic aptamer
gggagacaagaauaaagcgaguuagcugugucaugcuaguuaaccucucacuuaagagucgaugaugcuuagcuag

SEQ ID NO: 183 - RNA, 75 nt, artificial sequence
  synthetic aptamer
gggagacaagaauaaagcgaguuaaacuagugcuaacucaacacacuuaauaaagagucgaugaugcuuagcuag

SEQ ID NO: 184 - RNA, 76 nt, artificial sequence
  synthetic aptamer
gggagacaagaauaaagcgaguuacgcuaugucaaacuagcuaacuacaacacaagagucgaugaugcuuagcuag

SEQ ID NO: 185 - RNA, 76 nt, artificial sequence
  synthetic aptamer
gggagacaagaauaaagcgaguuuccacaaagcuaaaccagcuaacuuaucacaagagucgaugaugcuuagcuag

SEQ ID NO: 186 - RNA, 76 nt, artificial sequence
  synthetic aptamer
gggagacaagaauaaagcgaguuugcuauugucauacuaguuaacagaaucacaagagucgaugaugcuuagcuag

SEQ ID NO: 187 - RNA, 76 nt, artificial sequence
  synthetic aptamer
gggagacaagaauaaagcgaguuuccacaaagucaaacuaaccagcacuucacaagagucgaugaugcuuagcuag

SEQ ID NO: 188 - RNA, 76 nt, artificial sequence
  synthetic aptamer
gggagacaagaauaaagcgaguugcgcauuacgaaucuacaccaaagggucggaagagucgaugaugcuuagcuag

SEQ ID NO: 189 - RNA, 76 nt, artificial sequence
  synthetic aptamer
gggagacaagaauaaagcgaguuucuucaaaucacuccuaguuaacaugucacaagagucgaugaugcuuagcuag

SEQ ID NO: 190 - RNA, 75 nt, artificial sequence; identical to SEQ ID NO: 103
  synthetic aptamer

SEQ ID NO: 191 - RNA, 76 nt, artificial sequence
  synthetic aptamer
gggagacaagaauaaagcgaguuuguauucuuuucaaucuagcaacuuaucacaagagucgaugaugcuuagcuag

SEQ ID NO: 192 - RNA, 76 nt, artificial sequence
  synthetic aptamer
gggagacaagaauaaagcgaguuugcguugucaaucuagcuuucaucaacacuaagagucgaugaugcuuagcuag

SEQ ID NO: 193 - RNA, 76 nt, artificial sequence
  synthetic aptamer
gggagacaagaauaaagcgaguuacgcuuugucaaacuaauuaacuacaucacaagagucgaugaugcuuagcuag

SEQ ID NO: 194 - RNA, 76 nt, artificial sequence
  synthetic aptamer
gggagacaagaauaaagcgaguuucagcaaccaauaccuagcuuccaucacacaagagucgaugaugcuuagcuag

SEQ ID NO: 195 - RNA, 76 nt, artificial sequence
  synthetic aptamer
gggagacaagaauaaagcgaguuauauucugucaaucuaguuuccaucucacuaagagucgaugaugcuuagcuag

SEQ ID NO: 196 - RNA, 76 nt, artificial sequence
  synthetic aptamer
gggagacaagaauaaagcgaguuuccuccaaccaacucuagcucacuuaucacaagagucgaugaugcuuagcuag

SEQ ID NO: 197 - RNA, 76 nt, artificial sequence
  synthetic aptamer
gggagacaagaauaaagcgaguuaucguuucgucaaacuagguaacauaucacaagagucgaugaugcuuagcuag

SEQ ID NO: 198 - RNA, 76 nt, artificial sequence
  synthetic aptamer
gggagacaagaauaaagcgaguuugcucucucaacaccuaucuaacuagucacaagagucgaugaugcuuagcuag

SEQ ID NO: 199 - RNA, 76 nt, artificial sequence
  synthetic aptamer
gggagacaagaauaaagcgaguuagcuaugucaaacuagcucuccuuaucacaaagagucgaugaugcuuagcuag

SEQ ID NO: 200 - RNA, 76 nt, artificial sequence
  synthetic aptamer
gggagacaagaauaaagcgaguuuccacgauccaucaacuagcaacuuaucacaagagucgaugaugcuuagcuag

SEQ ID NO: 201 - RNA, 76 nt, artificial sequence
  synthetic aptamer
gggagacaagaauaaagcgaguuuccacgacagcaaucuagcucucauaucacaagagucgaugaugcuuagcuag

SEQ ID NO: 202 - RNA, 76 nt, artificial sequence
  synthetic aptamer
gggagacaagaauaaagcgaguuuaauuguugucaaacuaacuuccauaucacaagagucgaugaugcuuagcuag

SEQ ID NO: 203 - RNA, 76 nt, artificial sequence
  synthetic aptamer
gggagacaagaauaaagcgaguuauauucugacaaacuagcuaacgacauuacaagagucgaugaugcuuagcuag

SEQ ID NO: 204 - RNA, 76 nt, artificial sequence
  synthetic aptamer
gggagacaagaauaaagcgaguugugcuuccaaaauucuaacuaacuuaucacaagagucgaugaugcuuagcuag

SEQ ID NO: 205 - RNA, 76 nt, artificial sequence
  synthetic aptamer
gggagacaagaauaaagcgaguuagccauugucaaacuaguucacacaaucacaagagucgaugaugcuuagcuag

SEQ ID NO: 206 - RNA, 76 nt, artificial sequence
  synthetic aptamer
gggagacaagaauaaagcgaguuugaucucucgccaacuaguuaacuuaucacaagagucgaugaugcuuagcuag

SEQ ID NO: 207 - RNA, 76 nt, artificial sequence
  synthetic aptamer
gggagacaagaauaaagcgaguugcaagaaaacacgagcacauuuaccuacaaaagagucgaugaugcuuagcuag

SEQ ID NO: 208 - RNA, 76 nt, artificial sequence
  synthetic aptamer
gggagacaagaauaaagcgaguugcaacuaaauauagcacgccgacauuguaaaagagucgaugaugcuuagcuag

SEQ ID NO: 209 - RNA, 76 nt, artificial sequence
  synthetic aptamer
gggagacaagaauaaagcgaguuugcaugugucacacuaguuaacacgaucacaagagucgaugaugcuuagcuag

SEQ ID NO: 210 - RNA, 76 nt, artificial sequence
  synthetic aptamer
gggagacaagaauaaagcgaguuugcugugucauucuaagcuaacauagucacaagagucgaugaugcuuagcuag

SEQ ID NO: 211 - RNA, 76 nt, artificial sequence
  synthetic aptamer
gggagacaagaauaaagcgaguuuacgcucugucauacuuucuaacuuaucacaagagucgaugaugcuuagcuag

SEQ ID NO: 212 - RNA, 76 nt, artificial sequence
  synthetic aptamer
gggagacaagaauaaagcgaguucuauucugucauacuagcuaacacuucacaaagagucgaugaugcuuagcuag

SEQ ID NO: 213 - RNA, 76 nt, artificial sequence
  synthetic aptamer
gggagacaagaauaaagcgaguucacucagacguucacauaacgcuguucgauaagagucgaugaugcuuagcuag

SEQ ID NO: 214 - RNA, 76 nt, artificial sequence
  synthetic aptamer
gggagacaagaauaaagcgaguucuuccuaaacugccgaaacugcacaauaucaagagucgaugaugcuuagcuag

SEQ ID NO: 215 - RNA, 76 nt, artificial sequence
  synthetic aptamer
gggagacaagaauaaagcgaguuggcauuauuacguacucaccauacgcuaauaagagucgaugaugcuuagcuag

SEQ ID NO: 216 - RNA, 76 nt, artificial sequence
  synthetic aptamer
gggagacaagaauaaagcgaguugaucugauuucaaguugucauugcauaaucaagagucgaugaugcuuagcuag

SEQ ID NO: 217 - RNA, 76 nt, artificial sequence
  synthetic aptamer
gggagacaagaauaaagcgaguuugcuaugucaaucuagcuuccaucagcacuaagagucgaugaugcuuagcuag

SEQ ID NO: 218 - RNA, 76 nt, artificial sequence
  synthetic aptamer
gggagacaagaauaaagcgaguuacacuaacaggcucucuucucuacuacaguaagagucgaugaugcuuagcuag

SEQ ID NO: 219 - RNA, 76 nt, artificial sequence
  synthetic aptamer
gggagacaagaauaaagcgaguucacgcucugucaaucuaucuaacuuaucacaagagucgaugaugcuuagcuag

SEQ ID NO: 220 - RNA, 76 nt, artificial sequence
  synthetic aptamer
gggagacaagaauaaagcgaguuguauucuuucgcacuagccaacauaaucacaagagucgaugaugcuuagcuag

SEQ ID NO: 221 - RNA, 76 nt, artificial sequence
  synthetic aptamer
gggagacaagaauaaagcgaguuacgcucaugucauacuaauuaacauaucacaagagucgaugaugcuuagcuag

SEQ ID NO: 222 - RNA, 76 nt, artificial sequence
  synthetic aptamer
gggagacaagaauaaagcgaguuggacuuccaaacaucucguuaacacaucacaagagucgaugaugcuuagcuag

SEQ ID NO: 223 - RNA, 76 nt, artificial sequence; identical to SEQ ID NO: 100
  synthetic aptamer

SEQ ID NO: 224 - RNA, 76 nt, artificial sequence; identical to SEQ ID NO: 166
  synthetic aptamer

SEQ ID NO: 225 - RNA, 76 nt, artificial sequence
  synthetic aptamer
gggagacaagaauaaagcgaguugaacuagcaucaaucucuacaauacauuggaagagucgaugaugcuuagcuag

SEQ ID NO: 226 - RNA, 76 nt, artificial sequence; identical to SEQ ID NO: 216
  synthetic aptamer

SEQ ID NO: 227 - RNA, 75 nt, artificial sequence
  synthetic aptamer
gggagacaagaauaaagcgaguuggccuuacauuguacauaauucauaugucaagagucgaugaugcuuagcuag

SEQ ID NO: 228 - RNA, 76 nt, artificial sequence; identical to SEQ ID NO: 104
  synthetic aptamer

SEQ ID NO: 229 - RNA, 76 nt, artificial sequence
  synthetic aptamer
gggagacaagaauaaagcgaguuccgcagugcaaggcucaaggucuaccacugaagagucgaugaugcuuagcuag

SEQ ID NO: 230 - RNA, 189 nt, artificial sequence
  synthetic aptamer
gggagacaagaauaaagcgaguugaugcauuuagccaggauaguccccuauaguaauaauugaugcauuuagccaggauagucccuauaguaauaauugaugcauuuagccaggauaguccccuauaguaauaauugaugcauuuagccaggauaguucccauaguaagagucgaugaugcuuagcuag

SEQ ID NO: 231 - RNA, 76 nt, artificial sequence
  synthetic aptamer
gggagacaagaauaaagcgaguuccguagugcaaggcucaaggucuaccacugaagagucgaugaugcuuagcuag

SEQ ID NO: 232 - RNA, 76 nt, artificial sequence
  synthetic aptamer
gggagacaagaauaaagcgaguuuucaccacaaacaacuaacuaacuuaucacaagagucgaugaugcuuagcuag

SEQ ID NO: 233 - RNA, 76 nt, artificial sequence
  synthetic aptamer
gggagacaagaauaaagcgaguuggccuuccaucaaacagcuuucaauaucacaagagucgaugaugcuuagcuag

SEQ ID NO: 234 - RNA, 76 nt, artificial sequence; identical to SEQ ID NO: 173
  synthetic aptamer

SEQ ID NO: 235 - RNA, 77 nt, artificial sequence
  synthetic aptamer
gggagacaagaauaaagcgaguuuuuacguucuucaagacuugacugucuaugaaagagucgaugaugcuuagcuag

SEQ ID NO: 236 - RNA, 76 nt, artificial sequence
  synthetic aptamer
gggagacaagaauaaagcgaguuauccacaaaucaaccaaguuaacacaucacaagagucgaugaugcuuagcuag

SEQ ID NO: 237 - RNA, 76 nt, artificial sequence
  synthetic aptamer
gggagacaagaauaaagcgaguuggccuuccaucaaacugcuuucaauaucacaagagucgaugaugcuuagcuag

SEQ ID NO: 238 - RNA, 76 nt, artificial sequence
  synthetic aptamer
gggagacaagaauaaagcgaguuuccacaaagccaaaccaacuaacuuaucacaagagucgaugaugcuuagcuag

SEQ ID NO: 239 - RNA, 76 nt, artificial sequence
  synthetic aptamer
gggagacaagaauaaagcgaguuuccgcacaaagcaacuagcuaacauaucacaagagucgaugaugcuuagcuag

SEQ ID NO: 240 - RNA, 77 nt, artificial sequence
  synthetic aptamer
gggagacaagaauaaagcgaguuuccacacauccaaacuagucgacuuaucacaaagagucgaugaugcuuagcuag

SEQ ID NO: 241 - RNA, 76 nt, artificial sequence
  synthetic aptamer
gggagacaagaauaaagcgaguuuccacaaauaaaaucuaguucacuuaucacaagagucgaugaugcuuagcuag

SEQ ID NO: 242 - RNA, 76 nt, artificial sequence; identical to SEQ ID NO: 174
  synthetic aptamer

SEQ ID NO: 243 - RNA, 76 nt, artificial sequence
  synthetic aptamer
gggagacaagaauaaagcgaguuccuucaaaucaaaccuaacuaacuuaucacaagagucgaugaugcuuagcuag

SEQ ID NO: 244 - RNA, 76 nt, artificial sequence; identical to SEQ ID NO: 113
  synthetic aptamer

SEQ ID NO: 245 - RNA, 75 nt, artificial sequence
  synthetic aptamer
gggagacaagaauaaagcgaguuuuccucacaucaaucagcuaacuuaucacaagagucgaugaugcuuagcuag

SEQ ID NO: 246 - RNA, 76 nt, artificial sequence
  synthetic aptamer
gggagacaagaauaaagcgaguuuccacaaauccaaccuagcaaacccaucacaagagucgaugaugcuuagcuag

SEQ ID NO: 247 - RNA, 76 nt, artificial sequence
  synthetic aptamer
gggagacaagaauaaagcgaguuuccacacauccaaacuagacaacuuaucacaagagucgaugaugcuuagcuag

SEQ ID NO: 248 - RNA, 76 nt, artificial sequence; identical to SEQ ID NO: 129
  synthetic aptamer

SEQ ID NO: 249 - RNA, 76 nt, artificial sequence
  synthetic aptamer
gggagacaagaauaaagcgaguuuccacgcaaacaaacuaucuuacacaucacaagagucgaugaugcuuagcuag

SEQ ID NO: 250 - RNA, 76 nt, artificial sequence
  synthetic aptamer
gggagacaagaauaaagcgaguuugucauaucuaaggcacauauaccuauccaaagagucgaugaugcuuagcuag

SEQ ID NO: 251 - RNA, 76 nt, artificial sequence
  synthetic aptamer
gggagacaagaauaaagcgaguuuccacgaaauacaucccagcuagcaaucacaagagucgaugaugcuuagcuag

SEQ ID NO: 252 - RNA, 76 nt, artificial sequence
  synthetic aptamer
gggagacaagaauaaagcgaguuugcauugucauauuagcuaacaucaacacaaagagucgaugaugcuuagcuag

SEQ ID NO: 253 - RNA, 76 nt, artificial sequence; identical to SEQ ID NO: 119
  synthetic aptamer

SEQ ID NO: 254 - RNA, 76 nt, artificial sequence
  synthetic aptamer
gggagacaagaauaaagcgaguuuccacaaauaccaucuaacuaacaaaucacaagagucgaugaugcuuagcuag

SEQ ID NO: 255 - RNA, 76 nt, artificial sequence
  synthetic aptamer
gggagacaagaauaaagcgaguugcuuuugucaaacuagcuaucacuaucacaaagagucgaugaugcuuagcuag

SEQ ID NO: 256 - RNA, 76 nt, artificial sequence
  synthetic aptamer
gggagacaagaauaaagcgaguuuccacaaaucaacacagucuaacuuaucacaagagucgaugaugcuuagcuag

SEQ ID NO: 257 - RNA, 76 nt, artificial sequence; identical to SEQ ID NO: 162
  synthetic aptamer

SEQ ID NO: 258 - RNA, 76 nt, artificial sequence
  synthetic aptamer
gggagacaagaauaaagcgaguuuccacuaaucaucacuagcucacacaucacaagagucgaugaugcuuagcuag

SEQ ID NO: 259 - RNA, 76 nt, artificial sequence
  synthetic aptamer
gggagacaagaauaaagcgaguucucagcacaaccaacuagcucacuaaucacaagagucgaugaugcuuagcuag

SEQ ID NO: 260 - RNA, 76 nt, artificial sequence
  synthetic aptamer
gggagacaagaauaaagcgaguuucagcaaacaaccacuuucuaacauaucacaagagucgaugaugcuuagcuag

SEQ ID NO: 261 - RNA, 76 nt, artificial sequence
  synthetic aptamer
gggagacaagaauaaagcgaguuuccacaauucaacacuaacuaacuuaucacaagagucgaugaugcuuagcuag

SEQ ID NO: 262 - RNA, 76 nt, artificial sequence
  synthetic aptamer
gggagacaagaauaaagcgaguuucaccaacaacaucuaucuaacuuaucacaaagagucgaugaugcuuagcuag

SEQ ID NO: 263 - RNA, 76 nt, artificial sequence
  synthetic aptamer
gggagacaagaauaaagcgaguuuccuuacacacacacacguuaacuaaucacaagagucgaugaugcuuagcuag

SEQ ID NO: 264 - RNA, 76 nt, artificial sequence
  synthetic aptamer
gggagacaagaauaaagcgaguuuccacaagccaaaccuaacuaacauaucgcaagagucgaugaugcuuagcuag

SEQ ID NO: 265 - RNA, 76 nt, artificial sequence
  synthetic aptamer
gggagacaagaauaaagcgaguuucagcacacacaaacuagccaacuaaucacaagagucgaugaugcuuagcuag

SEQ ID NO: 266 - RNA, 76 nt, artificial sequence
  synthetic aptamer
gggagacaagaauaaagcgaguuguauucuugcaaccuagguaacuucucacaaagagucgaugaugcuuagcuag

SEQ ID NO: 267 - RNA, 76 nt, artificial sequence
  synthetic aptamer
gggagacaagaauaaagcgaguuagcuuagucaaacuaacuaaccucaacacuaagagucgaugaugcuuagcuag

SEQ ID NO: 268 - RNA, 76 nt, artificial sequence
  synthetic aptamer
gggagacaagaauaaagcgaguuuccacauccagcaacuaacuaacuuaucacaagagucgaugaugcuuagcuag

SEQ ID NO: 269 - RNA, 76 nt, artificial sequence
  synthetic aptamer
gggagacaagaauaaagcgaguuauauucugucacacuagcuaacuuaucacaaagagucgaugaugcuuagcuag

SEQ ID NO: 270 - RNA, 76 nt, artificial sequence
  synthetic aptamer
gggagacaagaauaaagcgaguuuccucacaccaaacuaucuuccaacaucacaagagucgaugaugcuuagcuag

SEQ ID NO: 271 - RNA, 76 nt, artificial sequence; identical to SEQ ID NO: 184
  synthetic aptamer

SEQ ID NO: 272 - RNA, 76 nt, artificial sequence
  synthetic aptamer
gggagacaagaauaaagcgaguuauauucuugcauacuaucuaacaucaacacaagagucgaugaugcuuagcuag

SEQ ID NO: 273 - RNA, 76 nt, artificial sequence
  synthetic aptamer
gggagacaagaauaaagcgaguuuccacgcaaacaaaacuaguuagcacucacaagagucgaugaugcuuagcuag

SEQ ID NO: 274 - RNA, 76 nt, artificial sequence
  synthetic aptamer
gggagacaagaauaaagcgaguuucuucacauaacaacuagcucucaaaucacaagagucgaugaugcuuagcuag

SEQ ID NO: 275 - RNA, 76 nt, artificial sequence
  synthetic aptamer
gggagacaagaauaaagcgaguuucuucacgcaaaaccuaucuaacuuaucacaagagucgaugaugcuuagcuag

SEQ ID NO: 276 - RNA, 76 nt, artificial sequence
  synthetic aptamer
gggagacaagaauaaagcgaguuaccacaaaucaacacuaguuaacacaucacaagagucgaugaugcuuagcuag

SEQ ID NO: 277 - RNA, 76 nt, artificial sequence
  synthetic aptamer
gggagacaagaauaaagcgaguuagcuuugucaaucuaguuuaacuucuucgcaagagucgaugaugcuuagcuag

SEQ ID NO: 278 - RNA, 76 nt, artificial sequence
  synthetic aptamer
gggagacaagaauaaagcgaguuuccacgguccacaacuaucuaacauaucacaagagucgaugaugcuuagcuag

SEQ ID NO: 279 - RNA, 76 nt, artificial sequence
  synthetic aptamer
gggagacaagaauaaagcgaguuuccuugacaacaaacuaacuaacuuaucacaagagucgaugaugcuuagcuag

SEQ ID NO: 280 - RNA, 76 nt, artificial sequence
  synthetic aptamer
gggagacaagaauaaagcgaguuuuccucacaaccacaaaucuaacuuaucacaagagucgaugaugcuuagcuag

SEQ ID NO: 281 - RNA, 76 nt, artificial sequence
  synthetic aptamer
gggagacaagaauaaagcgaguuguccuuuaauucuuuggcuaacuaaaucacaagagucgaugaugcuuagcuag

SEQ ID NO: 282 - RNA, 76 nt, artificial sequence
  synthetic aptamer
gggagacaagaauaaagcgaguuguauucuugcacacuagccuccaucacaccaagagucgaugaugcuuagcuag

SEQ ID NO: 283 - RNA, 76 nt, artificial sequence
  synthetic aptamer
gggagacaagaauaaagcgaguuuccgcaaaacaaaccuagcaaacuuaucacaagagucgaugaugcuuagcuag

SEQ ID NO: 284 - RNA, 76 nt, artificial sequence
  synthetic aptamer
gggagacaagaauaaagcgaguuucuucaaauacacacuaguuaacuauucacaagagucgaugaugcuuagcuag

SEQ ID NO: 285 - RNA, 76 nt, artificial sequence
  synthetic aptamer
gggagacaagaauaaagcgaguuauacaauucuacauagcgcgccuaccaacuaagagucgaugaugcuuagcuag

SEQ ID NO: 286 - RNA, 76 nt, artificial sequence
  synthetic aptamer
gggagacaagaauaaagcgaguuguucuuccauccaacagcucucgauaucacaagagucgaugaugcuuagcuag

SEQ ID NO: 287 - RNA, 76 nt, artificial sequence
  synthetic aptamer
gggagacaagaauaaagcgaguuuuccucaaaucauaauggcuaacuuaucacaagagucgaugaugcuuagcuag

SEQ ID NO: 288 - RNA, 76 nt, artificial sequence
  synthetic aptamer
gggagacaagaauaaagcgaguuugcucucucaacaccuaucuaacuaaucacaagagucgaugaugcuuagcuag

SEQ ID NO: 289 - RNA, 76 nt, artificial sequence
  synthetic aptamer
gggagacaagaauaaagcgaguuucuacgcaaccaaacuaguuuacauuucacaagagucgaugaugcuuagcuag

SEQ ID NO: 290 - RNA, 76 nt, artificial sequence
  synthetic aptamer
gggagacaagaauaaagcgaguugaucuaaacauuucucauaacaaauuguucaagagucgaugaugcuuagcuag

SEQ ID NO: 291 - RNA, 76 nt, artificial sequence
  synthetic aptamer
gggagacaagaauaaagcgaguuuccaccaaacaaucuaucuaacuaauucacaagagucgaugaugcuuagcuag

SEQ ID NO: 292 - RNA, 76 nt, artificial sequence
  synthetic aptamer
gggagacaagaauaaagcgaguuuccacaaacacaaucuaacuagcucaacacaagagucgaugaugcuuagcuag

SEQ ID NO: 293 - RNA, 76 nt, artificial sequence
  synthetic aptamer
gggagacaagaauaaagcgaguuguauucuugcgaucuagcuaucauauucacaagagucgaugaugcuuagcuag

SEQ ID NO: 294 - RNA, 76 nt, artificial sequence
  synthetic aptamer
gggagacaagaauaaagcgaguucccacaaaccaucaacagcuaauauuucacaagagucgaugaugcuuagcuag

SEQ ID NO: 295 - RNA, 76 nt, artificial sequence
  synthetic aptamer
gggagacaagaauaaagcgaguuggccuuuacacauuaaagcacuacguuauaaagagucgaugaugcuuagcuag

SEQ ID NO: 296 - RNA, 76 nt, artificial sequence
  synthetic aptamer
gggagacaagaauaaagcgaguuuguauuccuucauacuaacuaacuuaucacaagagucgaugaugcuuagcuag

SEQ ID NO: 297 - RNA, 76 nt, artificial sequence
  synthetic aptamer
gggagacaagaauaaagcgaguuuccacaaaaucaaucuaacuaacauaucacaagagucgaugaugcuuagcuag

SEQ ID NO: 298 - RNA, 76 nt, artificial sequence
  synthetic aptamer
gggagacaagaauaaagcgaguuuucacacacacacacuaacuuucacaucacaagagucgaugaugcuuagcuag

SEQ ID NO: 299 - RNA, 76 nt, artificial sequence
  synthetic aptamer
gggagacaagaauaaagcgaguuuccacacaccucaacuaacuaacagaucacaagagucgaugaugcuuagcuag

SEQ ID NO: 300 - RNA, 76 nt, artificial sequence
  synthetic aptamer
gggagacaagaauaaagcgaguuguauuacugucauacuaguuaacuuaucacaagagucgaugaugcuuagcuag

SEQ ID NO: 301 - RNA, 76 nt, artificial sequence
  synthetic aptamer
gggagacaagaauaaagcgaguuuccaccaauccaacuaucucacgccaucacaagagucgaugaugcuuagcuag

SEQ ID NO: 302 - RNA, 76 nt, artificial sequence
  synthetic aptamer
gggagacaagaauaaagcgaguuuucacaucaaccaucuagcuuucaaaucacaagagucgaugaugcuuagcuag

SEQ ID NO: 303 - RNA, 76 nt, artificial sequence
  synthetic aptamer
gggagacaagaauaaagcgaguuuccacaaauccaaucuagcaaacccaucacaagagucgaugaugcuuagcuag

SEQ ID NO: 304 - RNA, 76 nt, artificial sequence
  synthetic aptamer
gggagacaagaauaaagcgaguuucuucaaccaacaauuagcucucuuaucacaagagucgaugaugcuuagcuag

SEQ ID NO: 305 - RNA, 76 nt, artificial sequence
  synthetic aptamer
gggagacaagaauaaagcgaguuucaucaugcauaaucuagcuaacuuaucacaagagucgaugaugcuuagcuag

SEQ ID NO: 306 - RNA, 76 nt, artificial sequence
  synthetic aptamer
gggagacaagaauaaagcgaguuguucuuccaucaacaaguuaacuacaucacaagagucgaugaugcuuagcuag

SEQ ID NO: 307 - RNA, 74 nt, artificial sequence
  synthetic aptamer
gggagacaagaauaaagcgaguuuguauuuucgaacuuucuaacacaucacaagagucgaugaugcuuagcuag

SEQ ID NO: 308 - RNA, 75 nt, artificial sequence; identical to SEQ ID NO: 183
  synthetic aptamer

SEQ ID NO: 309 - RNA, 76 nt, artificial sequence
  synthetic aptamer
gggagacaagaauaaagcgaguuuccaccacaauccacuaguuuucauaucacaagagucgaugaugcuuagcuag

SEQ ID NO: 310 - RNA, 76 nt, artificial sequence; identical to SEQ ID NO: 214
  synthetic aptamer

SEQ ID NO: 311 - RNA, 76 nt, artificial sequence; identical to SEQ ID NO: 217
  synthetic aptamer

SEQ ID NO: 312 - RNA, 76 nt, artificial sequence
  synthetic aptamer
gggagacaagaauaaagcgaguuuugccgucaauaugcauguuaucgucuguaaagagucgaugaugcuuagcuag

SEQ ID NO: 313 - RNA, 76 nt, artificial sequence
  synthetic aptamer
gggagacaagaauaaagcgaguuccguagugcgaggcucaaggucuaccacugaagagucgaugaugcuuagcuag

SEQ ID NO: 314 - RNA, 76 nt, artificial sequence
  synthetic aptamer
gggagacaagaauaaagcgaguuacaaacaauggcgugcucaugaucguguuaaagagucgaugaugcuuagcuag

SEQ ID NO: 315 - RNA, 76 nt, artificial sequence
  synthetic aptamer
gggagacaagaauaaagcgaguucuauuugcaaacaccaagacccucuacgugaagagucgaugaugcuuagcuag

SEQ ID NO: 316 - RNA, 76 nt, artificial sequence
  synthetic aptamer
gggagacaagaauaaagcgaguugaucugauuucaaguuaucauugcauaaucaagagucgaugaugcuuagcuag

SEQ ID NO: 317 - RNA, 76 nt, artificial sequence
  synthetic aptamer
gggagacaagaauaaagcgaguuacccuguuuuuaccuaacuagcauaucacuaagagucgaugaugcuuagcuag

SEQ ID NO: 318 - RNA, 76 nt, artificial sequence
  synthetic aptamer
gggagacaagaauaaagcgaguuuccacaaaugccuccuagcuaacuuaucacaagagucgaugaugcuuagcuag

SEQ ID NO: 319 - RNA, 76 nt, artificial sequence
  synthetic aptamer
gggagacaagaauaaagcgaguuucggcacaauccaucuaguuaacauaucacaagagucgaugaugcuuagcuag

SEQ ID NO: 320 - RNA, 76 nt, artificial sequence
  synthetic aptamer
gggagacaagaauaaagcgaguuucacgcauugaucacaguacauuaggauucaagagucgaugaugcuuagcuag

SEQ ID NO: 321 - RNA, 75 nt, artificial sequence
  synthetic aptamer
gggagacaagaauaaagcgaguuggccuuacauguacuauaauucauaugucaagagucgaugaugcuuagcuag

SEQ ID NO: 322 - RNA, 76 nt, artificial sequence
  synthetic aptamer
gggagacaagaauaaagcgaguuuccacaagccaaaccuaacuaacauaucacaagagucgaugaugcuuagcuag

SEQ ID NO: 323 - RNA, 76 nt, artificial sequence
  synthetic aptamer
gggagacaagaauaaagcgaguuucaccaaccacacccuaguuuucauaucacaagagucgaugaugcuuagcuag

SEQ ID NO: 324 - RNA, 76 nt, artificial sequence
  synthetic aptamer
gggagacaagaauaaagcgaguucaugccuuauauuacuugcagaguaguaucaagagucgaugaugcuuagcuag

SEQ ID NO: 325 - RNA, 76 nt, artificial sequence
  synthetic aptamer
gggagacaagaauaaagcgaguuugcuaugucauuccaguucacaucuucacaaagagucgaugaugcuuagcuag

SEQ ID NO: 326 - RNA, 76 nt, artificial sequence
  synthetic aptamer
gggagacaagaauaaagcgaguuucauucuggucgaaacaguuaacaugucacaagagucgaugaugcuuagcuag

SEQ ID NO: 327 - RNA, 76 nt, artificial sequence; identical to SEQ ID NO: 167
  synthetic aptamer

SEQ ID NO: 328 - RNA, 76 nt, artificial sequence
  synthetic aptamer
gggagacaagaauaaagcgaguuguauaucuuaacacaagcgagguaauuugcaagagucgaugaugcuuagcuag

(19) United States
(12) Patent Application Publication
(10) Pub. No.: US 2009/0081679 A1
(43) Pub. Date: Mar. 26, 2009

(54) Compositions and methods for in vivo SELEX

(76) Inventors: Anthony Dominic Keefe, Cambridge, MA (US); Charles Wilson, Concord, MA (US); Chunhua Wang, Acton, MA (US)

Correspondence Address: MINTZ, LEVIN, COHN, FERRIS, GLOVSKYAND POPEO, P.C., ONE FINANCIAL CENTER, BOSTON, MA, 02111, US

(21) Appl. No.: 12/220,327

(22) Filed: Jul. 21, 2008

Related U.S. Application Data
(60) Provisional application No. 60/961,305, filed on Jul. 20, 2007.

(51) Int. Cl. C12Q 1/68

(52) U.S. Cl. 435/6

(57) ABSTRACT

The invention describes methods for the in vivo selection of oligonucleotides, preferably aptamers, that persist in biological compartments. In one embodiment, the biological compartment comprises at least one tissue which, in a preferred embodiment, is the blood within the circulatory system of a living mammal. The invention also contemplates oligonucleotides, preferably aptamers, selected through in vivo SELEX that may be linked to therapeutic or diagnostic compositions including other oligonucleotides.

### RELATED APPLICATIONS

This non-provisional patent application claims the benefit under 35 U.S.C. § 119(e) to U.S. Provisional application 60/961,305, filed Jul. 20, 2007, which is herein incorporated by reference in its entirety.

### FIELD OF INVENTION

The invention relates generally to the field of nucleic acids and more particularly to methods for selecting oligonucleotides, preferably aptamers, retained in biological compartments, wherein, the oligonucleotides, preferably aptamers, are useful in selected embodiments as therapeutics, diagnostics, payload delivery and in target validation. In preferred embodiments, the invention relates to compositions and methods for the selection of oligonucleotides, preferably aptamers, which persist in the blood for extended periods of time.

### BACKGROUND OF THE INVENTION

An aptamer by definition is an isolated nucleic acid molecule that binds with high specificity and affinity to some target, such as a protein, small molecule, carbohydrate, peptide or any other biological molecule, through interactions other than Watson-Crick base pairing.

Aptamers, like peptides generated by phage display or antibodies, are capable of specifically binding to selected targets and modulating the target's activity or binding interactions, e.g., through binding, aptamers may block or activate their target's ability to function. As with antibodies, this functional property of specific binding to a target, is an inherent property. Also as with antibodies, although the skilled person may not know what precise structural characteristics an aptamer to a target will have, the skilled person knows how to identify, make and use such a molecule in the absence of a precise structural definition.

Traditionally discovered by an in vitro selection process know as: “Systematic Evolution of Ligands by Exponential Enrichment” (“SELEX”) from pools of random sequence oligonucleotides, aptamers have been generated for hundreds of proteins, including growth factors, transcription factors, enzymes, immunoglobulins and receptors. A typical aptamer is 5-15 kDa in size (15-45 nucleotides), binds its target with nanomolar to sub-nanomolar affinity and discriminates against closely related targets (e.g., aptamers will typically not bind other proteins from the same gene family). A series of structural studies have shown that aptamers are capable of using the same types of binding interactions (e.g., hydrogen bonding, electrostatic complementarities, hydrophobic contacts, steric exclusion) that drive affinity and specificity in antibody-antigen complexes.

Aptamers have a number of desirable characteristics for use as therapeutics and diagnostics including high specificity and affinity, biological efficacy and excellent pharmacokinetic properties. In addition, they offer specific competitive advantages over antibodies and other protein biologics.

A challenge faced by many drugs is selecting a drug that can persist in a biological compartment for a desired amount of time before the drug is metabolized to substantially inactive metabolites (or is cleared from the body). What is needed, therefore, is a method for selecting oligonucleotides, preferably aptamers, that can reside for a desired period of time in a variety of biological compartments including, but not limited to, the circulatory system. The present invention provides materials and methods to meet these and other needs.

### SUMMARY OF THE INVENTION

Embodiments of the present invention describe methods for selecting oligonucleotides, preferably aptamers, that persist for a given period of time in biological compartments. In some embodiments, the biological compartment is within a multicellular organism, for example, a living multicellular organism. In one embodiment, the multicellular organism is a mammal.

In some embodiments, it is contemplated that the methods for selecting oligonucleotides, preferably aptamers, that persist in a specific organ comprise the steps of: i) preparing a library of oligonucleotides, ii) introducing the library of oligonucleotides into a biological compartment of a living organism, iii) waiting for a period of time to elapse, iv) harvesting the biological compartment (or portion thereof), v) collecting oligonucleotides, of the library of oligonucleotides, from the biological compartment and vi) amplifying the oligonucleotides contained therein. In one embodiment, this process is repeated iteratively until an enriched population of oligonucleotides, preferably aptamers, is identified. In one embodiment, the iterative repetitions of in vivo SELEX is in a range of three to nine rounds. As used, herein, “living organism” comprises: plants, animals, fungi, protists, archaea and bacteria. In a preferred embodiment a living organism is a mammal.

In some embodiments, it is contemplated that the methods for selecting oligonucleotides, preferably aptamers, that persist in a specific organ comprises the steps of: i) preparing a library of oligonucleotides, ii) introducing the library of oligonucleotides into an artery of a living organism that perfuses an organ, iii) waiting for a period of time to elapse sufficient to allow the oligonucleotide to perfuse the organ, iv) harvesting the oligonucleotide perfused organ (or a portion thereof), v) collecting oligonucleotides, of the library of oligonucleotides, from the oligonucleotide perfused organ and v) amplifying the oligonucleotides contained therein. In one embodiment, the organ is selected from the group consisting of kidney, liver and spleen. In one embodiment, this process is repeated iteratively until an enriched population of oligonucleotides, preferably aptamers, is identified. In one embodiment, the iterative repetitions of in vivo SELEX is in a range of three to nine rounds.

In some embodiments, it is contemplated that the method for selecting oligonucleotides that persist in a specific organ comprises the steps of: i) preparing a library of oligonucleotides, ii) introducing the library of oligonucleotides into an artery of a living organism that perfuses an organ, iii) waiting for a period of time to elapse sufficient to allow the oligonucleotide to perfuse the organ, iv) collecting oligonucleotides of the library of oligonucleotides from a sample of blood collected from at least one vein that drains the organ and v) amplifying the oligonucleotides contained therein. In one embodiment, the organ is the kidney, the artery is the renal artery and the vein is the renal vein. In one embodiment, this process is repeated iteratively until an enriched population of oligonucleotides, preferably aptamers, is identified. In one embodiment, the iterative repetitions of in vivo SELEX is in a range of three to nine rounds.

In some embodiments, the biological compartment is a tissue. While it is not intended that the methods of the present invention be limited to any specific tissue, in some embodiments the tissue is selected from the group consisting of: epithelial tissue, connective tissue, muscle tissue, nervous tissue. In a preferred embodiment, the tissue is blood.

In some embodiments, it is contemplated the tissue is tumor tissue. In some embodiments, the tumor tissue is benign. In some embodiments, the tumor is malignant.

In some embodiments, the method for selecting oligonucleotides, preferably aptamers, that persist in a biological compartment comprises the steps of: i) preparing a library of oligonucleotides, ii) introducing the library of oligonucleotides into the circulatory system of a living organism, iii) waiting for a period of time to elapse sufficient to allow the oligonucleotides to distribute throughout the circulatory system, iv) collecting oligonucleotides of the library of oligonucleotides from a sample of blood collected from the living organism and v) amplifying the oligonucleotides, preferably aptamers, contained therein. In some embodiments, this process is repeated iteratively until an enriched population of oligonucleotides, preferably aptamers, is identified. For clarity, an enriched population of oligonucleotides, preferably aptamers, is characterized by individual species of oligonucleotides, preferably aptamers, that persist in a biological compartment as compared to the unselected oligonucleotide library. In one embodiment, the iterative repetitions of in vivo SELEX is in a range of three to nine rounds.

As used above, oligonucleotides distributed: “throughout the circulatory system” include oligonucleotides that are retained in the circulatory system from the time they are introduced, in step ii), into the circulatory system until the time they are collected, in step iv), from the blood and oligonucleotides that leave the circulatory system after they are introduced, in step ii), reside in a biological compartment outside the circulatory system and then return to the circulatory system prior to the blood sample taken in step iv).

In another embodiment, the method for selecting oligonucleotides, preferably aptamers, that persist in a biological compartment comprises the steps of: i) preparing a library of nuclease-stabilized oligonucleotides, ii) introducing the library of nuclease-stabilized oligonucleotides into the circulatory system of a living organism, iii) waiting for a period of time to elapse sufficient to allow the oligonucleotides to distribute throughout the circulatory system, iv) collecting oligonucleotides of the library of oligonucleotides from a sample of blood collected from the living organism and v) amplifying the oligonucleotides contained therein. In one embodiment, this process is repeated iteratively until an enriched population of oligonucleotides, preferably aptamers, is identified. In one embodiment, the iterative repetitions of in vivo SELEX is in a range of three to nine rounds. More specifically, an enriched population of oligonucleotides, preferably aptamers, wherein, individual species of oligonucleotides, preferably aptamers, have a greater tendency to persist in the blood as compared to the unselected oligonucleotide library.

As used above, oligonucleotides distributed: “throughout the circulatory system” include oligonucleotides that are retained in the circulatory system from the time they are introduced, in step ii), into the circulatory system until the time they are collected, in step iv), from the blood and oligonucleotides that leave the circulatory system after they are introduced, in step ii), reside in a biological compartment outside the circulatory system and then return to the circulatory system prior to the blood sample taken in step iv).

In another embodiment, the method for selecting oligonucleotides, preferably aptamers, that persist in a biological compartment comprises the steps of: i) preparing a library of nuclease stabilized MNA oligonucleotides, ii) introducing the library of nuclease stabilized MNA oligonucleotides into the circulatory system of a living organism, iii) waiting for a period of time to elapse sufficient to allow the oligonucleotides to distribute throughout the circulatory system, iv) collecting oligonucleotides of the library of oligonucleotides from a sample of blood collected from the living organism and v) amplifying the oligonucleotides contained therein. In one embodiment, this process is repeated iteratively until an enriched population of oligonucleotides, preferably aptamers, is identified. In one embodiment, the iterative repetitions of in vivo SELEX is in a range of three to nine rounds. More specifically, an enriched population of oligonucleotides, preferably aptamers, wherein, individual species of MNA oligonucleotides, preferably aptamers, have a greater tendency to persist in the blood as compared to the unselected oligonucleotide library.

As used above, oligonucleotides distributed: “throughout the circulatory system” include oligonucleotides that are retained in the circulatory system from the time they are introduced, in step ii), into the circulatory system until the time they are collected, in step iv), from the blood and oligonucleotides that leave the circulatory system after they are introduced, in step ii), reside in a biological compartment outside the circulatory system and then return to the circulatory system prior to the blood sample taken in step iv).

In some embodiments, it is contemplated that the in vivo selections described herein are performed under conditions such that bivalent aptamers are generated. In one embodiment, two aptamers are selected via in vivo SELEX against different targets and subsequently linked to form a single bivalent aptamer. While it is not intended that the present invention be limited to any specific mechanism, these bivalent aptamers would bind at least two different biological molecules, wherein one binding event mediates a therapeutic effect while the second binding event mediates the retention of the bivalent aptamer in a biological compartment. In one embodiment, it is contemplated that the bivalent aptamer is dosed less frequently or in smaller amounts, and has a higher potency and/or a reduced likelihood of side-effects.

In some embodiments, the first binding event modulates one or more biological activities and/or biological functions of a given target. In some embodiments, the first binding event modulates the expression of the target. In some embodiments, the first target is a biological target. In some embodiments, the biological target is an extracellular target. In some embodiments, the extracellular target is a target that internalizes the bivalent aptamer during or after the first binding event. In some embodiments, the target is a biological target, molecule, drug or other compound that resides in a biological compartment such as the circulatory system.

In some embodiments, an oligonucleotide, preferably an aptamer that persists in a biological compartment (i.e., a biological compartment-persistent oligonucleotide, preferably biological compartment-persistent aptamer) is identified using the in vivo SELEX methods provided herein, and the biological compartment-persistent oligonucleotide, preferably an aptamer, is linked to a second oligonucleotide, preferably a second aptamer. In some embodiments, the biological compartment-persistent oligonucleotide, preferably an aptamer, and the second oligonucleotide, preferably a second aptamer, bind to, or otherwise interact with the same target. In other embodiments, the biological compartment-persistent oligonucleotide, preferably an aptamer, and the second oligonucleotide, preferably a second aptamer, bind to, or otherwise interact with at least two different biological targets. The second oligonucleotide, preferably a second aptamer, is, by way of non-limiting example, a known oligonucleotide, preferably aptamer, sequence; an oligonucleotide, preferably aptamer, sequence identified using the in vivo SELEX methods described herein, and/or an oligonucleotide, preferably aptamer, sequence identified using an in vitro SELEX method (e.g., those described in U.S. patent application Ser. No. 07/536,428, filed Jun. 11, 1990, now abandoned, U.S. Pat. No. 5,475,096 entitled “Nucleic Acid Ligands” and U.S. Pat. No. 5,270,163 (see also WO 91/19813) entitled “Nucleic Acid Ligands”), including, but not limited to, an in vitro 2′-modified SELEX method. The biological compartment-persistent oligonucleotide, preferably an aptamer, is linked to the second oligonucleotide, preferably an aptamer, using any of a variety of art-recognized methods for linking oligonucleotides, including, but not limited, covalent linkages, non-covalent linkages, use of a nucleic acid or non-nucleic acid linking moiety.

In some embodiments, the biological compartment-persistent oligonucleotide, preferably biological compartment-persistent aptamer, is identified using the in vivo SELEX methods provided herein and is linked to a second moiety, preferably not an aptamer moiety. By way of non-limiting example, the biological compartment-persistent oligonucleotide, preferably aptamer, is linked to a peptide or polypeptide moiety such as, for example, an immunoglobulin or functional portion thereof; a toxin moiety such as, for example, an immunotoxin; an oligonucleotide moiety such as, for example, an antisense oligonucleotide, siRNA molecule or other interfering RNA molecule; and/or a small molecule. The biological compartment-persistent oligonucleotide, preferably an aptamer, is linked to the second moiety using any of a variety of art-recognized methods for linking oligonucleotides, including, but not limited, covalent linkages, non-covalent linkages, use of a nucleic acid or non-nucleic acid linking moiety.

While it is not intended that the present invention be limited to any specific mechanism, the in vivo selection of oligonucleotides, preferably aptamers, described by the present invention may be tuned to select for oligonucleotides, preferably aptamers, that have a desired profile of functional characteristics. In embodiments of in vivo SELEX wherein oligonucleotides, preferably aptamers, are selected for persistence in a given organ or tissue (but are spared exposure to the renal circulation) it is expected these oligonucleotides, preferably aptamers, will be: i) nuclease resistant and ii) will not cross-over into another tissue or organ.

In embodiments of in vivo SELEX wherein oligonucleotides, preferably aptamers, are selected for persistence in the blood and, therefore, are exposed to the renal circulation these oligonucleotides, preferably aptamers, are: i) nuclease resistant, ii) resistant to renal clearance and iii) in some embodiments are not bound or taken up by other tissues or organs.

As used herein, these methodologies are referred to as: “in vivo SELEX”. It is contemplated that the oligonucleotides, preferably aptamers, generated from in vivo SELEX will be useful as therapeutics, diagnostics, for extending half life and in target validation. In one embodiment, the biological compartment is an organ comprising at least one tissue. In a preferred embodiment, the tissue is blood. In another embodiment, the present invention contemplates oligonucleotides, preferably aptamers, selected for persistence in biological compartments, wherein the oligonucleotides, preferably aptamers, are linked to compositions that facilitate the therapeutic and/or diagnostic applications of the oligonucleotides, preferably aptamers.

The in vivo SELEX methods described by the present invention select for oligonucleotides that persist in biological compartment. In some embodiments, these oligonucleotides are aptamers.

It is also contemplated that one skilled in the art would be able apply variations of in vitro SELEX (including, but not limited to, counter SELEX, toggle SELEX and agonist SELEX) to the methods for in vivo SELEX described in the instant application.

### BRIEF DESCRIPTION OF THE DRAWINGS

FIG. 1 is a schematic representation of the traditional in vitro aptamer selection (SELEX) process from pools of random sequence oligonucleotides.

FIG. 2 is an illustration depicting various PEGylation strategies representing standard mono-PEGylation, multiple PEGylation and oligomerization via PEGylation.

FIG. 3 is an illustration of a 40 kDa branched PEG.

FIG. 4 is a graph that depicts the relationship between blood from each time-point and the number of cycles of PCR that were required to reach an arbitrary amplicon concentration. The number of cycles of PCR was then used to calculate the original MNA concentration in the blood samples at each time point.

### DETAILED DESCRIPTION OF THE INVENTION

The details of one or more embodiments of the invention are set forth in the accompanying description below. Although any methods and materials similar or equivalent to those described herein can be used in the practice or testing of the present invention, the preferred methods and materials are now described. Other features, objects and advantages of the invention will be apparent from the description. In the specification, the singular forms also include the plural unless the context clearly dictates otherwise. Unless defined otherwise, all technical and scientific terms used herein have the same meaning as commonly understood by one of ordinary skill in the art to which this invention belongs. In the case of conflict, the present specification will control.

As used herein, oligonucleotides distributed: “throughout the circulatory system” include oligonucleotides that are retained in the circulatory system from the time they are introduced into the circulatory system until the time they are collected from the blood and oligonucleotides that leave the circulatory system after they are introduced, reside in a biological compartment outside the circulatory system and then return to the circulatory system prior to being sampled in the blood.

In some embodiments of the present invention, a methodology generally known as SELEX is used as a tool in support of the novel in vivo SELEX methods described herein, the in vivo SELEX generating oligonucleotides, preferably aptamers, that persist in biological compartments. The SELEX technology is described in the following sections. Certain terms used to describe the invention herein are defined as follows.

It is also contemplated that one skilled in the art would be able apply variations of in vitro SELEX (including, but not limited to, counter SELEX, toggle SELEX and agonist SELEX) to the methods for in vivo SELEX described in the instant application.

### The SELEX Method

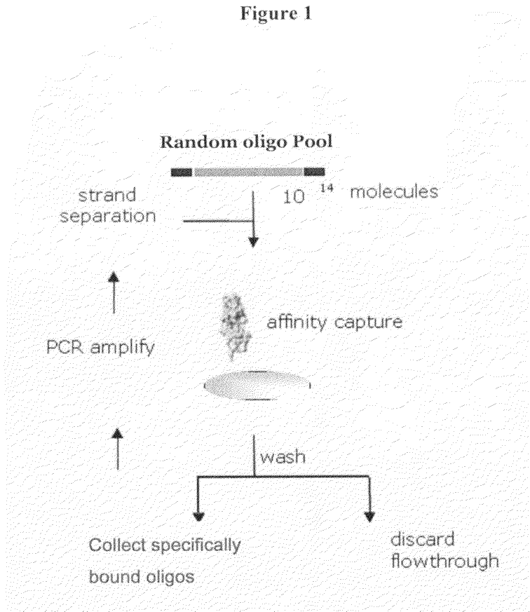

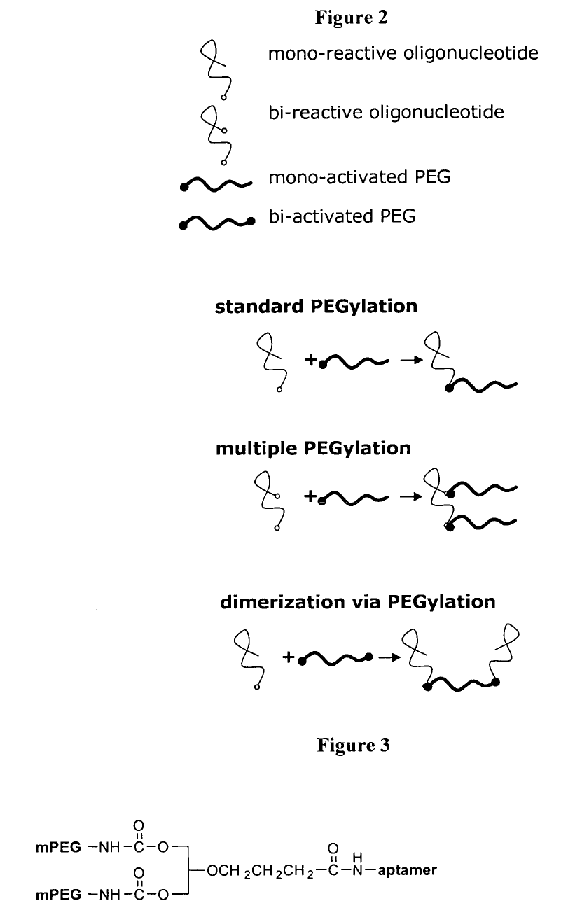

The preferred method for generating an aptamer, generally depicted in FIG. 1, is with a process entitled “Systematic Evolution of Ligands by Exponential Enrichment” (“SELEX”). The SELEX process, a method for the in vitro evolution of nucleic acid molecules with highly specific binding to target molecules, is described in, e.g., U.S. patent application Ser. No. 07/536,428, filed Jun. 11, 1990, now abandoned, U.S. Pat. No. 5,475,096 entitled “Nucleic Acid Ligands” and U.S. Pat. No. 5,270,163 (see also WO 91/19813) entitled “Nucleic Acid Ligands”. In vivo SELEX expands upon the SELEX methodology by performing iterative cycles of selection in vivo to obtain oligonucleotides (which in some embodiments are aptamers) that persist in a biological compartment (as compared to a unselected oligonucleotide library).

The in vivo SELEX process is based on the unique insight that oligonucleotides, preferably aptamers, have sufficient capacity for forming a variety of two- and three-dimensional structures and sufficient chemical versatility available within their monomers to act as ligands (i.e., form specific binding pairs) with virtually any chemical compound, whether monomeric or polymeric. Molecules of any size or composition can serve as targets. Targets may be found within a biological compartment. As used, herein, a “biological compartment” include: organs, tissues, extracellular matrices, organelles, cytosol, and biological fluids. Examples of organs include, but are not limited to, heart, lung, brain, eye, stomach, spleen, bone, pancreas, kidney, liver, intestine, skin, urinary bladder, ovary, uterus and testicle. Examples of tissues include, but are not limited to, epithelial tissue, connective tissue (which includes blood, bone and cartilage), muscle tissue and nervous tissue. Examples of extracellular matrices include, but are not limited to, the interstitial matrix and the basement membrane. Examples of organelles include, but are not limited to, the mitochondria, the Golgi apparatus, endoplasmic reticulum, vacuoles, microsomes, plasma membrane and nucleus. Examples of biological fluids include, but are not limited to, allantoic, amniotic, bronchioalveolar, cerebrospinal, extracellular, extravascular, interstitial, intraocular, lymph, pleural and synovial.

The in vivo SELEX process selects for oligonucleotides (which in some embodiments are aptamers) which are not cleared from the organism and/or substantially metabolized within the milieu of a biological compartment. Oligonucleotides, preferably aptamers, obtained through the in vivo SELEX procedure will thus have the property of persistence in a biological compartment.

In vivo SELEX relies as a starting point upon a large library or pool of single stranded oligonucleotides comprising randomized sequences. The oligonucleotides can be modified or unmodified DNA, RNA, or DNA/RNA hybrids. In some examples, the pool comprises 100% random or partially random oligonucleotides. That is to say, in some examples the pool comprises 100% degenerate or partially degenerate oligonucleotides. In other examples, the pool comprises random or partially random oligonucleotides containing at least one fixed sequence and/or conserved sequence incorporated within randomized sequence. In other examples, the pool comprises random or partially random oligonucleotides containing at least one fixed sequence and/or conserved sequence at its 5′ and/or 3′ end which may comprise a sequence shared by all the molecules of the oligonucleotide pool. Fixed sequences are sequences common to oligonucleotides in the pool which are incorporated for a preselected purpose such as, CpG motifs described further below, hybridization sites for PCR primers, promoter, or initiation, sequences for RNA polymerases (e.g., T3, T4, T7 and SP6), restriction sites, or homopolymeric sequences, such as poly A or poly T tracts, catalytic cores, sites for selective binding to affinity columns and other sequences to facilitate cloning and/or sequencing of an oligonucleotide of interest. Conserved sequences are sequences, other than the previously described fixed sequences, shared by a number of aptamers that bind to the same target.

The oligonucleotides of the pool preferably include a randomized sequence portion as well as fixed sequences necessary for efficient amplification. Typically the oligonucleotides of the starting pool contain fixed 5′ and 3′ terminal sequences which flank an internal region of 30-50 random nucleotides. The randomized nucleotides can be produced in a number of ways including chemical synthesis and size selection from randomly cleaved cellular nucleic acids. Sequence variation in test nucleic acids can also be introduced or increased by mutagenesis, or recombination, before or during the selection/amplification iterations.

The random sequence portion of the oligonucleotide can be of any length and can comprise ribonucleotides and/or deoxyribonucleotides and can include modified or non-natural nucleotides or nucleotide analogs. See, e.g., U.S. Pat. No. 5,958,691; U.S. Pat. No. 5,660,985; U.S. Pat. No. 5,958,691; U.S. Pat. No. 5,698,687; U.S. Pat. No. 5,817,635; U.S. Pat. No. 5,672,695 and PCT Publication WO 92/07065. Random oligonucleotides can be synthesized from activated nucleosides using solid phase oligonucleotide synthesis techniques well known in the art. See, e.g., Froehler et al., Nucl. Acid Res. 14:5399-5467 (1986) and Froehler et al., Tet. Lett. 27:5575-5578 (1986). Random oligonucleotides can also be synthesized using solution phase methods such as triester synthesis methods. See, e.g., Sood et al., Nucl. Acid Res. 4:2557 (1977) and Hirose et al., Tet. Lett., 28:2449 (1978). Typical syntheses carried out on automated DNA synthesis equipment yield 10¹⁴-10¹⁷ individual molecules, a number sufficient for most in vivo SELEX experiments. Sufficiently large regions of random sequence in the sequence design increase the likelihood that each synthesized molecule is likely to represent a unique sequence.

The starting library of oligonucleotides may be generated by automated chemical synthesis on a DNA synthesizer. To synthesize randomized sequences, mixtures of all four nucleotides are added at each nucleotide addition step during the synthesis process, allowing for random incorporation of nucleotides. As stated above, in one embodiment, random oligonucleotides comprise entirely random sequences; however, in other embodiments, random oligonucleotides can comprise stretches of nonrandom or partially random sequences. Partially random sequences can be created by adding the four activated nucleosides in different molar ratios at each addition step.

The starting library of oligonucleotides may be either RNA, DNA, or substituted RNA or DNA. In those instances where an RNA library is to be used as the starting library it is typically generated by synthesizing a DNA library, optionally PCR amplifying, then transcribing the DNA library in vitro using T7 RNA polymerase or modified T7 RNA polymerases, or other polymerases and purifying the transcribed library. The RNA or DNA library is then mixed with the target under conditions favorable for binding and subjected to step-wise iterations of binding, partitioning and amplification, using the same general selection scheme, to achieve virtually any desired criterion of binding affinity and selectivity. More specifically, starting with a mixture containing the starting pool of oligonucleotides, the in vivo SELEX method includes steps of: a) preparing a candidate mixture of oligonucleotides; b) introducing the candidate mixture of oligonucleotides into a biological compartment of a living organism; c) partitioning the oligonucleotides having an increased persistence in the biological compartment from the remainder of the candidate mixture and d) amplifying the oligonucleotides having an increased persistence in the biological compartment to yield a mixture of oligonucleotides, preferably aptamers, enriched for oligonucleotides, preferably aptamers, with relatively greater persistence in a biological compartment than the remainder of the candidate mixture and (e) reiterating the steps of b), c) and d) through as many cycles as desired to yield oligonucleotides, preferably aptamers, that persist in a biological compartment. In those instances where RNA oligonucleotides, preferably aptamers, are being selected, the in vivo SELEX method further comprises the steps of: (i) reverse transcribing the oligonucleotides from step c) before amplification in step (d) and (ii) transcribing the amplified oligonucleotides from step (d) before restarting the process. As used, herein, “partitioning” refers to the process of dissociating the oligonucleotides, from a library or candidate mixture of oligonucleotides, associated with a biological compartment (or portion thereof) under conditions such that the partitioned oligonucleotides may then be subsequently sequenced and/or amplified.

Within an oligonucleotide mixture containing a large number of possible sequences and structures, there is a wide range of binding affinities for a given biological compartment. An oligonucleotide mixture comprising, for example, a 20 nucleotide randomized segment can have 4²⁰ candidate possibilities. After partitioning and amplification, a second oligonucleotide mixture is generated, enriched for oligonucleotides which persist in a biological compartment(s). Additional rounds of selection progressively favor the best oligonucleotides, preferably aptamers, until the resulting oligonucleotide mixture is predominantly composed of only one or a few sequences. These can then be cloned, sequenced and individually tested.

Cycles of selection and amplification are repeated until a desired goal is achieved. In the most general case, selection/amplification is continued until no significant improvement in persistence in a biological compartment is achieved on repetition of the cycle. The method is typically used to sample approximately 10¹⁴ different oligonucleotide species but may be used to sample as many as about 10¹⁸ different oligonucleotides species. Generally, oligonucleotides, preferably aptamers, are selected in a 5 to 20 cycle procedure. In one embodiment, heterogeneity is introduced only in the initial selection stages and does not intentionally occur throughout the replicating process.

In many cases, it is not necessarily desirable to perform the iterative steps of in vivo SELEX until a single oligonucleotide, preferably aptamer, is identified. The biological compartment persistent oligonucleotide, preferably aptamer, solution may include a family of oligonucleotide, preferably aptamer, structures or motifs that have a number of conserved sequences and a number of sequences which can be substituted or added without significantly affecting the persistence of the oligonucleotide, preferably aptamer, in the biological compartment. By terminating the SELEX process before it has converged on a single sequence, it is possible to determine the sequence of a number of members of the oligonucleotide, preferably aptamer, solution family.

A variety of oligonucleotide primary, secondary and tertiary structures are known to exist. The structures or motifs that have been shown most commonly to be involved in non-Watson-Crick type interactions are referred to as hairpin loops, symmetric and asymmetric bulges, pseudoknots and myriad combinations of the same. Almost all known cases of such motifs suggest that they can be formed in a oligonucleotide sequence of no more than 30 nucleotides. For this reason, it is often preferred that in vivo SELEX procedures with contiguous randomized segments be initiated with oligonucleotide sequences containing a randomized segment of between about 20 to about 50 nucleotides and in some embodiments, about 30 to about 40 nucleotides. In one example, the 5′-fixed:random-3′fixed sequence comprises a random sequence of about 30 to about 50 nucleotides.

Counter-in vivo SELEX is a method for improving the specificity of oligonucleotides, preferably aptamers, to a biological compartment by eliminating oligonucleotides, preferably aptamers, with cross-reactivity to one or more other biological compartments. Counter-in vivo SELEX is comprised of the steps of: a) preparing a candidate mixture of oligonucleotides; b) introducing the candidate mixture of oligonucleotides into a first biological compartment; c) partitioning the oligonucleotides having an increased persistence in the first biological compartment from the remainder of the candidate mixture, (d) contacting the oligonucleotides with increased persistence in the first biological compartment with a second biological compartments such that the oligonucleotides, preferably aptamers, with specific affinity for the second biological compartment is removed and (e) amplifying the oligonucleotides demonstrating persistence in the first biological compartment to yield a mixture of oligonucleotides enriched for oligonucleotide, preferably aptamer, sequences with a relatively higher persistence in the first biological compartment affinity and specificity for binding to the target molecule.

One potential problem encountered in the use of oligonucleotides as therapeutics, diagnostic agents and vaccines is that oligonucleotides in their phosphodiester form may be quickly degraded in body fluids by intracellular and extracellular enzymes such as endonucleases and exonucleases before the desired effect is manifest. The in vivo SELEX method thus encompasses the identification of high-affinity oligonucleotides, preferably aptamers, containing modified nucleotides conferring improved characteristics on the oligonucleotide, preferably aptamer, such as increased resistance to nucleases. Examples of such modifications include chemical substitutions at the sugar and/or phosphate and/or base positions. SELEX-identified oligonucleotides containing modified nucleotides are described, e.g., in U.S. Pat. No. 5,660,985, which describes oligonucleotides containing nucleotide derivatives chemically modified at the 2′ position of ribose, 5 position of pyrimidines and 8 position of purines, U.S. Pat. No. 5,756,703 which describes oligonucleotides containing various 2′-modified pyrimidines and U.S. Pat. No. 5,580,737 which describes highly specific oligonucleotides containing one or more nucleotides modified with 2′-amino (2′—NH₂), 2′-fluoro (2′-F) and/or 2′-O-methyl (2′-OMe) substituents. In a preferred embodiment of the present invention, the nuclease resistant oligonucleotide is MNA.

Modifications of the oligonucleotides, preferably aptamers, contemplated in this invention include, but are not limited to, those which provide other chemical groups that incorporate additional charge, polarizability, hydrophobicity, hydrogen bonding, electrostatic interaction and fluxionality to the oligonucleotide, preferably aptamer, bases or to the oligonucleotide, preferably aptamer, as a whole. Modifications to generate oligonucleotide populations which are resistant to nucleases can also include one or more substitute internucleotide linkages, altered sugars, altered bases, or combinations thereof. Such modifications include, but are not limited to, 2′-position sugar modifications, 5-position pyrimidine modifications, 8-position purine modifications, modifications at exocyclic amines, substitution of 4-thiouridine, substitution of 5-bromo or 5-iodo-uracil; backbone modifications, phosphorothioate or alkyl phosphate modifications, methylations and unusual base-pairing combinations such as the isobases isocytidine and isoguanosine. Modifications can also include 3′ and 5′ modifications such as capping, e.g., addition of a 3′-3′-dT cap to increase exonuclease resistance (see, e.g., U.S. Pat. Nos. 5,674,685; 5,668,264; 6,207,816; and 6,229,002, each of which is incorporated by reference herein in its entirety).

In some embodiments, it is contemplated that oligonucleotides will be provided in which the P(O)O group is replaced by P(O)S (“thioate”), P(S)S (“dithioate”), P(O)NR₂ (“amidate”), P(O)R, P(O)OR′, CO or CH₂ (“formacetal”) or 3′-amine (—NH—CH₂—CH₂—), wherein each R or R′ is independently H or substituted or unsubstituted alkyl. Linkage groups can be attached to adjacent nucleotides through an —O—, —N— or —S— linkage. Not all linkages in the oligonucleotide are required to be identical.

In further embodiments, the oligonucleotides comprise modified sugar groups, for example, one or more of the hydroxyl groups is replaced with halogen, aliphatic groups, or functionalized as ethers or amines. In one embodiment, the 2′-position of the furanose residue is substituted by any of an O-methyl, O-alkyl, O-allyl, S-alkyl, S-allyl or halo group. Methods of synthesis of 2′-modified sugars are described, e.g., in Sproat, et al., Nucl. Acid Res. 19:733-738 (1991); Cotten, et al., Nucl. Acid Res. 19:2629-2635 (1991); and Hobbs, et al., Biochemistry 12:5138-5145 (1973). Other modifications are known to one of ordinary skill in the art. Such modifications may be pre-in vivo SELEX process modifications or post-in vivo SELEX process modifications (modification of previously identified unmodified oligonucleotides, preferably aptamers) or may be made by incorporation into the in vivo SELEX process.

Pre and Post in vivo SELEX process modifications or those made by incorporation into the SELEX process yield oligonucleotides, preferably aptamers, with improved stability, e.g., in vivo stability.

The in vivo SELEX method encompasses combining selected oligonucleotides with other selected oligonucleotides and non-oligonucleotide functional units as described in U.S. Pat. No. 5,637,459 and U.S. Pat. No. 5,683,867. The in vivo SELEX method further encompasses combining selected oligonucleotides, preferably aptamers, with lipophilic or non-immunogenic high molecular weight compounds in a diagnostic or therapeutic complex, as described, e.g., in U.S. Pat. No. 6,011,020, U.S. Pat. No. 6,051,698 and PCT Publication No. WO 98/18480. These patents and applications teach the combination of a broad array of shapes and other properties, with the efficient amplification and replication properties of oligonucleotides and with the desirable properties of other molecules.

### 2′ Modified SELEX

In order for an aptamer to be suitable for use as a therapeutic or diagnostic, it is preferably inexpensive to synthesize, safe and stable in vivo. Wild-type RNA and DNA aptamers are typically not stable in vivo because of their susceptibility to degradation by nucleases. Resistance to nuclease degradation can be greatly increased by the incorporation of modifying groups at the 2′-position.

2′-fluoro and 2′-amino groups have been successfully incorporated into oligonucleotide pools from which aptamers have been subsequently selected. However, these modifications greatly increase the cost of synthesis of the resultant aptamer and may introduce safety concerns in some cases because of the possibility that the modified nucleotides could be recycled into host DNA by degradation of the modified oligonucleotides and subsequent use of the nucleosides as substrates for DNA synthesis.

Oligonucleotides, preferably aptamers, that contain 2′-O-methyl (“2′-OMe”) nucleotides, as provided herein, overcome many of these drawbacks. Oligonucleotides containing 2′-OMe nucleotides are nuclease-resistant and inexpensive to synthesize. Although 2′-OMe nucleotides are ubiquitous in biological systems natural polymerases do not accept 2′-OMe NTPs as substrates under physiological conditions, thus there are no safety concerns over the recycling of 2′-OMe nucleosides into host DNA. SELEX™ methods used to generate 2′-modified aptamers are described, e.g., in U.S. Provisional Patent Application Ser. No. 60/430,761, filed Dec. 3, 2002, U.S. Provisional Patent Application Ser. No. 60/487,474, filed Jul. 15, 2003, U.S. Provisional Patent Application Ser. No. 60/517,039, filed Nov. 4, 2003, U.S. patent application Ser. No. 10/729,581, filed Dec. 3, 2003, U.S. patent application Ser. No. 10/873,856, filed Jun. 21, 2004, entitled “Method for in vitro Selection of 2′-O-methyl Substituted Nucleic Acids” and U.S. Provisional Patent Application Ser. No. 60/696,295, filed Jun. 30, 2005, entitled “Improved Materials and Methods for the Generation of Fully 2′-Modified Containing Nucleic Acid Transcripts”, each of which is herein incorporated by reference in its entirety.

The present invention includes oligonucleotides, preferably aptamers, that are selected for their capacity to persist in a biological compartment which, in a preferred embodiment, is the blood. These oligonucleotides, preferably aptamers, may contain modified nucleotides (e.g., nucleotides which have a modification at the 2′-position) to make the oligonucleotide more stable than the unmodified oligonucleotide to enzymatic and chemical degradation as well as thermal and physical degradation. Although there are several examples of 2′-OMe containing aptamers in the literature (see, e.g., Ruckman et al., J. Biol. Chem., 1998 273, 20556-20567-695) these were generated by the in vitro selection of libraries of modified transcripts in which the C and U residues were 2′-fluoro (2′-F) substituted and the A and G residues were 2′-OH. Once functional sequences were identified then each A and G residue was tested for tolerance to 2′-OMe substitution and the aptamer was re-synthesized having all A and G residues which tolerated 2′-OMe substitution as 2′-OMe residues. Most of the A and G residues of aptamers generated in this two-step fashion tolerate substitution with 2′-OMe residues, although, on average, approximately 20% do not. Consequently, oligonucleotides, preferably aptamers, generated using this method tend to contain from two to four 2′-OH residues and stability and cost of synthesis are compromised as a result. By incorporating modified nucleotides into the transcription reaction which generate stabilized oligonucleotides used in oligonucleotide pools from which aptamers are selected and enriched by SELEX (and/or any of its variations and improvements, including those described herein), the methods of the present invention eliminate the need for stabilizing the selected aptamer oligonucleotides (e.g., by resynthesizing the aptamer oligonucleotides with modified nucleotides).

It is contemplated that in some embodiments of the present invention oligonucleotides, preferably aptamers, comprising combinations of 2′-OH, 2′-F, 2′-deoxy and 2′-OMe modifications of the ATP, GTP, CTP, TTP and UTP nucleotides may be selected. In another embodiment, the present invention contemplates oligonucleotides, preferably aptamers, comprising combinations of 2′-OH, 2′-F, 2′-deoxy, 2′-OMe, 2′—NH₂ and 2′-methoxyethyl modifications of the ATP, GTP, CTP, TTP and UTP nucleotides. In a preferred embodiment, the present invention provides oligonucleotides, preferably aptamers, comprising all or substantially all 2′-OMe modified ATP, GTP, CTP, TTP and/or UTP nucleotides.

2′-modified oligonucleotides, preferably aptamers, of the invention are created using modified polymerases, e.g., a modified T7 polymerase, having a rate of incorporation of modified nucleotides having bulky substituents at the furanose 2′ position that is higher than that of wild-type polymerases. For example, a mutant T7 polymerase in which the tyrosine residue at position 639 has been changed to phenylalanine (Y639F) readily utilizes 2′deoxy, 2′amino- and 2′fluoro-nucleotide triphosphates (NTPs) as substrates and has been widely used to synthesize modified RNAs for a variety of applications. However, this mutant T7 polymerase reportedly can not readily utilize (i.e., incorporate) NTPs with bulky 2′-substituents such as 2′-OMe or 2′-azido (2′-N₃) substituents. For incorporation of bulky 2′ substituents, a mutant T7 polymerase having the histidine at position 784 changed to an alanine residue in addition to the Y639F mutation has been described (Y639F/H784A) and has been used in limited circumstances to incorporate modified pyrimidine NTPs. See Padilla, R. and Sousa, R., Nucleic Acids Res., 2002, 30 (24): 138. A mutant T7 RNA polymerase in which the tyrosine residue at position 639 has been changed to phenylalanine, the histidine residue at position 784 has been changed to an alanine and the lysine residue at position 378 has been changed to arginine (Y639F/H784A/K378R) has been used in limited circumstances to incorporate modified purine and pyrimidine NTPs, e.g., 2′-OMe NTPs, but requires a spike of 2′-OH GTP for transcription. See, Burmeister et. al., (2005) Chemistry and Biology, 12: 25-33. The K378R mutation is not near the active site of the polymerase and thus is believed to be a silent mutation, having no effect on the incorporation of 2′-OMe modified NTPs. A mutant T7 polymerase having the histidine at position 784 changed to an alanine residue (H784A) has also been described. Padilla et al., Nucleic Acids Research, 2002, 30: 138. In both the Y639F/H784A mutant and H784A mutant T7 polymerases, the change to a smaller amino acid residue such as alanine allows for the incorporation of bulkier nucleotide substrates, e.g., 2′-OMe substituted nucleotides. See, Chelliserry, K. and Ellington, A. D., (2004) Nature Biotech, 9:1155-60. Additional T7 RNA polymerases have been described with mutations in the active site of the T7 RNA polymerase which more readily incorporate bulky 2′-modified substrates, e.g., a mutant T7 RNA polymerase having the tyrosine residue at position 639 changed to a leucine (Y639L). However activity is often sacrificed for increased substrate specificity conferred by such mutations, leading to low transcript yields. See Padilla R and Sousa, R., (1999) Nucleic Acids Res., 27 (6): 1561.

Generally, it has been found that under the conditions disclosed herein, the Y693F mutant can be used for the incorporation of all 2′-OMe substituted NTPs except GTP and the Y639F/H784A, Y639F/H784A/K378R, Y639L/H784A, Y639L/H784A/K378R, Y639L, or the Y639L/K378R mutant T7 RNA polymerases can be used for the incorporation of all 2′-OMe substituted NTPs including GTP. It is expected that the H784A and H784A/K378R mutants possesses properties similar to the Y639F, Y639F/K378R, Y639F/H784A and the Y639F/H784A/K378R mutants when used under the conditions disclosed herein.

2′-modified oligonucleotides may be synthesized entirely of modified nucleotides, or with a subset of modified nucleotides. The modifications can be the same or different. Some or all nucleotides may be modified and those that are modified may contain the same modification. Some or all nucleotides may be modified and those that are modified may contain different modifications, e.g., all nucleotides containing the same base may have one type of modification, while nucleotides containing other bases may have different types of modification. All purine nucleotides may have one type of modification (or are unmodified), while all pyrimidine nucleotides have another, different type of modification (or are unmodified). In this way, transcripts, or pools of transcripts are generated using any combination of modifications, including for example, ribonucleotides (2′-OH), deoxyribonucleotides (2′-deoxy), 2′-amine nucleotides (2′—NH₂), 2′-fluoro nucleotides (2′-F) and 2′-O-methyl (2′-OMe) nucleotides. A transcription mixture containing 2′-OH A and G and 2′-F C and U is referred to as an “rRfY” mixture and aptamer selected there from are referred to as “rRfY” aptamers. A transcription mixture containing 2′-OMe A and G and 2′-F C and U is referred to as an “mRfY” mixture and aptamer selected there from are referred to as “rRfY” aptamers. A transcription mixture containing 2′-OMe C and U and 2′-OH A and G is referred to as an “rRmY” mixture and aptamers selected therefrom are referred to as “rRmY” aptamers. A transcription mixture containing deoxy A and G and 2′-OMe U and C is referred to as a “dRmY” mixture and aptamers selected therefrom are referred to as “dRmY” aptamers. A transcription mixture containing 2′-OMe A, C and U and 2′-OH G is referred to as a “rGmH” mixture and aptamers selected therefrom are referred to as “rGmH” aptamers. A transcription mixture alternately containing 2′-OMe A, C, U and G and 2′-OMe A, U and C and 2′-F G is referred to as an “alternating mixture” and aptamers selected therefrom are referred to as “alternating mixture” aptamers. A transcription mixture containing 2′-OMe A, U, C and G, where up to 10% of the G's are ribonucleotides is referred to as a “r/mGmH” mixture and aptamers selected therefrom are referred to as “r/mGmH” aptamers. A transcription mixture containing 2′-OMe A, U and C and 2′-F G is referred to as a “fGmH” mixture and aptamers selected therefrom are referred to as “fGmH” aptamers. A transcription mixture containing 2′-OMe A, U and C and deoxy G is referred to as a “dGmH” mixture and aptamers selected therefrom are referred to as “dGmH” aptamers. A transcription mixture containing deoxy A and 2′-OMe C, G and U is referred to as a “dAmB” mixture and aptamers selected therefrom are referred to as “dAmB” aptamers and a transcription mixture containing all 2′-OH nucleotides is referred to as a “rN” mixture and aptamers selected therefrom are referred to as “rN”, “rRrY” or “RNA” aptamers. A transcription mixture containing 2′-OH adenosine triphosphate and guanosine triphosphate and deoxy cytidine triphosphate and thymidine triphosphate is referred to as a rRdY mixture and aptamers selected therefrom are referred to as “rRdY’ aptamers. A “mRmY” (or “MNA”) aptamer is one containing only 2′-O-methyl nucleotides except for the starting nucleotide, which is 2′-OH guanosine or any wild type guanosine and may be derived from a r/mGmH oligonucleotide by post-SELEX replacement, when possible, of any 2′-OH Gs with 2′-OMe Gs. Alternatively, mRmY aptamers may be identified by mRmY SELEX

In one embodiment, any combination of 2′-OH, 2′-deoxy and 2′-OMe nucleotides is contemplated. Another embodiment contemplates any combination of 2′-deoxy and 2′-OMe nucleotides. Another embodiment contemplates any combination of 2′-deoxy and 2′-OMe nucleotides in which the pyrimidines are 2′-OMe (such as dRmY, mRmY or dGmH). In a preferred embodiment, the present invention describes methods for in vivo SELEX with nucleotides wherein all residues are 2′-methoxy (also referred to, herein, as MNA).

Incorporation of modified nucleotides into oligonucleotides, preferably aptamers, of the invention is accomplished before (pre-) the selection process (e.g., a pre-in vivo SELEX process modification). Optionally, oligonucleotides, preferably aptamers, of the invention in which modified nucleotides have been incorporated by pre-in vivo SELEX process modification can be further modified by a post-in vivo SELEX modification process (i.e., a post-SELEX process modification after in vivo SELEX). Pre-in vivo SELEX process modifications yield modified oligonucleotides, preferably aptamers, with in vivo stability. Post-in vivo SELEX process modifications, i.e., modification (e.g., truncation, deletion, substitution or additional nucleotide modifications of previously identified oligonucleotides, preferably aptamers, having nucleotides incorporated by pre-in vivo SELEX process modification) can result in a further improvement of in vivo stability.

To generate pools of 2′-modified (e.g., 2′-OMe) RNA transcripts in conditions under which a polymerase accepts 2′-modified NTPs the Y693F, Y693F/K378R, Y693F/H784A, Y693F/H784A/K378R, Y693L/H784A, Y693L/H784A/K378R, Y639L, Y639L/K378R, P266L, P266L/Y639F, P266L/Y639L, P266L/H784A, P266L/Y639F/H784A and the P266L/Y639L/H784A mutant T7 RNA polymerases can all be used. Other T7 RNA polymerases, particularly those that exhibit a high tolerance for bulky 2′-substituents, may also be used in the present invention. When used in a template-directed polymerization using the conditions disclosed herein, the Y639L/H784A, Y639L/H784A/K378R or the P266L/Y639L/H784A mutant T7 RNA polymerase can be used for the incorporation of all 2′-OMe NTPs, including GTP, with higher transcript yields than achieved by using the Y639F, Y639F/K378R, Y639F/H784A, Y639F/H784A/K378R, Y639L, Y639L/K378R, P266L, P266L/Y639F, P266L/Y639L, P266L/H784A, P266L/Y639F/H784A mutant T7 RNA polymerases. The Y639L/H784A, Y639L/H784A/K378R and the P266L/Y639L/H784A mutant T7 RNA polymerases can be used with but does not require 2′-OH GTP to achieve high yields of 2′-modified, e.g., 2′-OMe containing oligonucleotides.

In some embodiments, the r/mRmY transcription protocol using the Y639F/H874A mutant T7 RNAP was used. In a preferred embodiment a transcription protocol using the Y639L/H874A mutant T7 RNAP, which produced clones CLN9397 and CLN9406, was used.

Other polymerases, particularly those that exhibit a high tolerance for bulky 2′-substituents, may also be used in the present invention. Such polymerases can be screened for this capability by assaying their ability to incorporate modified nucleotides under the transcription conditions disclosed herein.

A number of factors have been determined to be important for the transcription conditions useful in the methods disclosed herein. For example, a leader sequence incorporated into the fixed sequence at the 5′ end of a DNA transcription template may be important to increase the yields of modified transcripts when the Y639F/K378R or Y639F/H784A/K378R mutant T7 RNA Polymerases are used for transcription, e.g., under the dRmY or r/mGmH transcription conditions described below. Additionally, a leader sequence may be used but is not necessary to help increase the yield of modified transcripts when the Y639L/H784A/K378R mutant T7 RNA polymerase is used for transcription, e.g., under the mRmY transcription conditions described below. The leader sequence is typically 6-15 nucleotides long and may be composed of all purines, or a mixture of purine and pyrimidine nucleotides.

Another important factor in obtaining transcripts incorporating modified nucleotides is the presence or concentration of 2′-OH guanosine (e.g., GMP, GTP, or another non-2′-OMe non-triphosphate). Transcription can be divided into two phases: the first phase is initiation, during which an NTP is added to the 3′-hydroxyl end of GTP (or GMP, or another non-2′-OMe non-triphosphate) to yield a dinucleotide which is then extended by about 10-12 nucleotides; the second phase is elongation, during which transcription proceeds beyond the addition of the first about 10-12 nucleotides. It has been found that small amounts of 2′-OH GTP (or GMP, or another non-2′-OMe non-triphosphate) added to a transcription mixture containing an excess of 2′-OMe GTP are sufficient to enable the polymerase to initiate transcription using 2′-OH GTP (or GMP, guanosine, or another non-2′-OMe non-triphosphate). Thus for example, a dRmY transcription mixture (containing deoxy purines and 2′OMe pyrimidines) requires the addition of a small amount of GMP to enable the polymerase to initiate transcription, whereas in a r/mGmH transcription mixture (containing up to 10% 2′-OH GTP), a small amount of GMP can be added to the transcription mixture but is not required to enable the polymerase to initiate transcription, because 2′-OH GTP is already present in the transcription mixture. Once transcription enters the elongation phase the reduced discrimination between 2′-OMe and 2′-OH GTP and the excess of 2′-OMe GTP over 2′-OH GTP allows the incorporation of principally the 2′-OMe GTP.

As described immediately above, priming transcription with 2′-OH guanosine (e.g., GMP, GTP or another non-2′-OMe non-triphosphate) is important. This effect results from the specificity of the polymerase for the initiating nucleotide. As a result, the 5′-terminal nucleotide of any transcript generated in this fashion is likely to be 2′-OH G. The preferred concentration of GMP is 0.5 mM and even more preferably 1 mM. It has also been found that including PEG, preferably PEG-8000, in the transcription reaction is useful to maximize incorporation of modified nucleotides.

Another important factor in the incorporation of 2′-OMe substituted nucleotides into transcripts is the use of both divalent magnesium and manganese in the transcription mixture. Different combinations of concentrations of magnesium chloride and manganese chloride have been found to affect yields of 2′-O-methylated transcripts, the optimum concentration of the magnesium and manganese chloride being dependent on the concentration in the transcription reaction mixture of NTPs which complex divalent metal ions. To obtain the greatest yields of all 2′-O-methylated transcripts (i.e., all 2′-OMe A, C and U and about 90% of G nucleotides), concentrations of approximately 5 mM magnesium chloride and 1.5 mM manganese chloride are preferred when each NTP is present at a concentration of 0.5 mM. When the concentration of each NTP is 1.0 mM, concentrations of approximately 6.5 mM magnesium chloride and 2.0 mM manganese chloride are preferred. When the concentration of each NTP is 2.0 mM, concentrations of approximately 9.6 mM magnesium chloride and 2.9 mM manganese chloride are preferred. In any case, departures from these concentrations of up to two-fold still give significant amounts of modified transcripts.

For maximum incorporation of 2′-OMe ATP (100%), UTP (100%), CTP (100%) and GTP (˜90%) (“r/mGmH”) into transcripts the following conditions are preferred: HEPES buffer 200 mM, DTT 40 mM, spermidine 2 mM, PEG-8000 10% (w/v), Triton X-100 0.01% (w/v), MgCl₂ 5 mM (6.5 mM where the concentration of each 2′-OMe NTP is 1.0 mM), MnCl₂ 1.5 mM (2.0 mM where the concentration of each 2′-OMe NTP is 1.0 mM), 2′-OMe NTP (each) 500 μM (more preferably, 1.0 mM), 2′-OH GTP 30 μM, 2′-OH GMP 500 μM, pH 7.5, Y639F/H784A/K378R T7 RNA Polymerase 200 nM, inorganic pyrophosphatase 5 units/ml and an all-purine leader sequence of at least 8 nucleotides long. As used herein, one unit of inorganic pyrophosphatase is defined as the amount of enzyme that will liberate 1.0 mole of inorganic orthophosphate per min. at pH 7.2 and 25° C.

For maximum incorporation (100%) of 2′-OH GTP and 2′-OMe ATP, UTP and CTP (“rGmH”) into transcripts the following conditions are preferred: HEPES buffer 200 mM, DTT 40 mM, spermidine 2 mM, PEG-8000 10% (w/v), Triton X-100 0.01% (w/v), MgCl₂ 5 mM (9.6 mM where the concentration of each NTP is 2.0 mM), MnCl₂ 1.5 mM (2.9 mM where the concentration of each NTP is 2.0 mM), NTP (each) 500 μM (more preferably, 2.0 mM), 2′-OH GMP 1 mM, pH 7.5, Y639F/K378R T7 RNA Polymerase 200 nM, inorganic pyrophosphatase 5 units/ml and an all-purine leader sequence of at least 8 nucleotides long.

For maximum incorporation of 2′-OMe ATP (100%), 2′-OMe UTP (100%), 2′-OMe CTP (100%) and 2′-OMe GTP (100%) (“mRmY”) into transcripts the following conditions are preferred: HEPES buffer 200 mM, DTT 40 mM, spermidine 2 mM, PEG-8000 10% (w/v), Triton X-100 0.01% (w/v), MgCl₂ 8 mM, MnCl₂2.5 mM, 2′-OMe NTP (each) 1.5 mM, 2′-OH GMP 1 mM, pH 7.5, Y639L/H784A/K378R mutant T7 RNA Polymerase 200 nM, inorganic pyrophosphatase 5 units/ml and a leader sequence that increases the transcription yield under the derived transcription conditions. In one embodiment, the leader sequence is an all purine leader sequence. In another embodiment, the leader sequence is a mixture of purines and pyrimidines.

For maximum incorporation (100%) of 2′-OH ATP and GTP and 2′-OMe UTP and CTP (“rRmY”) into transcripts the following conditions are preferred: HEPES buffer 200 mM, DTT 40 mM, spermidine 2 mM, PEG-8000 10% (w/v), Triton X-100 0.01% (w/v), MgCl₂ 5 mM (9.6 mM where the concentration of each NTP is 2.0 mM), MnCl₂ 1.5 mM (2.9 mM where the concentration of each NTP is 2.0 mM), NTP (each) 500 μM (more preferably, 2.0 mM), 2′-OH GMP 1 mM, pH 7.5, Y639F/H784A/K378R T7 RNA Polymerase 200 nM, inorganic pyrophosphatase 5 units/ml and an all-purine leader sequence of at least 8 nucleotides long.

For maximum incorporation (100%) of deoxy ATP and GTP and 2′-OMe UTP and CTP (“dRmY”) into transcripts the following conditions are preferred: HEPES buffer 200 mM, DTT 40 mM, spermine 2 mM, spermidine 2 mM, PEG-8000 10% (w/v), Triton X-100 0.01% (w/v), MgCl₂ 9.6 mM, MnCl₂ 2.9 mM, NTP (each) 2.0 mM, 2′-OH GMP 1 mM, pH 7.5, Y639F/K3787R T7 RNA Polymerase 200 nM, inorganic pyrophosphatase 5 units/ml and an all-purine leader sequence of at least 8 nucleotides long.

For maximum incorporation (100%) of 2′-OMe ATP, UTP and CTP and 2′-F GTP (“fGmH”) into transcripts the following conditions are preferred: HEPES buffer 200 mM, DTT 40 mM, spermidine 2 mM, PEG-8000 10% (w/v), Triton X-100 0.01% (w/v), MgCl₂ 9.6 mM, MnCl₂ 2.9 mM, 2′-OMe NTP (each) 2.0 mM, 2′-OH GMP 1 mM, pH 7.5, Y639F/K378R T7 RNA Polymerase 200 nM, inorganic pyrophosphatase 5 units/ml and an all-purine leader sequence of at least 8 nucleotides long.

For maximum incorporation (100%) of deoxy ATP and 2′-OMe UTP, GTP and CTP (“dAmB”) into transcripts the following conditions are preferred: HEPES buffer 200 mM, DTT 40 mM, spermidine 2 mM, PEG-8000 10% (w/v), Triton X-100 0.01% (w/v), MgCl₂ 9.6 mM, MnCl₂ 2.9 mM, NTP (each) 2.0 mM, 2′-OH GMP 1 mM, pH 7.5, Y639F/K378R T7 RNA Polymerase 200 nM, inorganic pyrophosphatase 5 units/ml and an all-purine leader sequence of at least 8 nucleotides long.

For each of the above (a) transcription is preferably performed at a temperature of from about 20° C. to about 50° C., preferably from about 30° C. to 45° C. and more preferably at about 37° C. for a period of at least two hrs. and (b) 50-300 nM of a double stranded DNA transcription template is used (200 nM template is used in round 1 to increase diversity (300 nM template is used in dRmY transcriptions) and for subsequent rounds approximately 50 nM, a 1/10 dilution of an optimized PCR reaction, using conditions described herein, is used). In some embodiment, it is contemplated that the DNA transcription templates, below, may be used (where ARC254 and ARC256 transcribe under all 2′-OMe conditions and ARC255 transcribes under rRmY conditions).

SEQ ID NO: 1
5′-CATCGATGCTAGTCGTAACGATCCNNNNNNNNNNNNNNNNNNNNNNN
NNNNNNNCGAGAACGTTCTCTCCTCTCCCTATAGTGAGTCGTATTA-3′
SEQ ID NO: 2
5′-CATGCATCGCGACTGACTAGCCGNNNNNNNNNNNNNNNNNNNNNNNN
NNNNNNGTAGAACGTTCTCTCCTCTCCCTATAGTGAGTCGTATTA-3′
SEQ ID NO: 3
5′-CATCGATCGATCGATCGACAGCGNNNNNNNNNNNNNNNNNNNNNNNN
NNNNNNGTAGAACGTTCTCTCCTCTCCCTATAGTGAGTCGTATTA-3′

Under RNA transcription conditions, the transcription reaction mixture comprises 2′-OH adenosine triphosphates (ATP), 2′-OH guanosine triphosphates (GTP), 2′-OH cytidine triphosphates (CTP) and 2′-OH uridine triphosphates (UTP). The modified oligonucleotides produced using the rN transcription mixtures of the present invention comprise substantially all 2′-OH adenosine, 2′-OH guanosine, 2′-OH cytidine and 2′-OH uridine. In a preferred embodiment of rN transcription, the resulting modified oligonucleotides comprise a sequence where at least 80% of all adenosine nucleotides are 2′-OH adenosine, at least 80% of all guanosine nucleotides are 2′-OH guanosine, at least 80% of all cytidine nucleotides are 2′-OH cytidine and at least 80% of all uridine nucleotides are 2′-OH uridine. In a more preferred embodiment of rN transcription, the resulting modified oligonucleotides of the present invention comprise a sequence where at least 90% of all adenosine nucleotides are 2′-OH adenosine, at least 90% of all guanosine nucleotides are 2′-OH guanosine, at least 90% of all cytidine nucleotides are 2′-OH cytidine and at least 90% of all uridine nucleotides are 2′-OH uridine. In a most preferred embodiment of rN transcription, the modified oligonucleotides of the present invention comprise a sequence where 100% of all adenosine nucleotides are 2′-OH adenosine, 100% of all guanosine nucleotides are 2′-OH guanosine, 100% of all cytidine nucleotides are 2′-OH cytidine and 100% of all uridine nucleotides are 2′-OH uridine.

Under rRmY transcription conditions, transcription reaction mixture comprises 2′-OH adenosine triphosphates, 2′-OH guanosine triphosphates, 2′-O-methyl cytidine triphosphates and 2′-O-methyl uridine triphosphates. The modified oligonucleotides produced using the rRmY transcription mixtures of the present invention comprise substantially all 2′-OH adenosine, 2′-OH guanosine, 2′-O-methyl cytidine and 2′-O-methyl uridine. In one embodiment of the present invention, it is contemplated the resulting modified oligonucleotides would comprise a sequence where at least 80% of all adenosine nucleotides are 2′-OH adenosine, at least 80% of all guanosine nucleotides are 2′-OH guanosine, at least 80% of all cytidine nucleotides are 2′-O-methyl cytidine and at least 80% of all uridine nucleotides are 2′-O-methyl uridine. In a more preferred embodiment, the resulting modified oligonucleotides comprise a sequence where at least 90% of all adenosine nucleotides are 2′-OH adenosine, at least 90% of all guanosine nucleotides are 2′-OH guanosine, at least 90% of all cytidine nucleotides are 2′-O-methyl cytidine and at least 90% of all uridine nucleotides are 2′-O-methyl uridine In a most preferred embodiment, the resulting modified oligonucleotides comprise a sequence where 100% of all adenosine nucleotides are 2′-OH adenosine, 100% of all guanosine nucleotides are 2′-OH guanosine, 100% of all cytidine nucleotides are 2′-O-methyl cytidine and 100% of all uridine nucleotides are 2′-O-methyl uridine.

Under dRmY transcription conditions, the transcription reaction mixture comprises 2′-deoxy adenosine triphosphates, 2′-deoxy guanosine triphosphates, 2′-O-methyl cytidine triphosphates and 2′-O-methyl uridine triphosphates. The modified oligonucleotides produced using the dRmY transcription conditions of the present invention comprise substantially all 2′-deoxy adenosine, 2′-deoxy guanosine, 2′-O-methyl cytidine and 2′-O-methyl uridine. In one embodiment of the present invention, it is contemplated the resulting modified oligonucleotides would comprise a sequence where at least 80% of all adenosine nucleotides are 2′-deoxy adenosine, at least 80% of all guanosine nucleotides are 2′-deoxy guanosine, at least 80% of all cytidine nucleotides are 2′-O-methyl cytidine and at least 80% of all uridine nucleotides are 2′-O-methyl uridine. In one embodiment of the present invention, it is contemplated the resulting modified oligonucleotides would comprise a sequence where at least 90% of all adenosine nucleotides are 2′-deoxy adenosine, at least 90% of all guanosine nucleotides are 2′-deoxy guanosine, at least 90% of all cytidine nucleotides are 2′-O-methyl cytidine and at least 90% of all uridine nucleotides are 2′-O-methyl uridine. In one embodiment of the present invention, it is contemplated the resulting modified oligonucleotides of the present invention would comprise a sequence where 100% of all adenosine nucleotides are 2′-deoxy adenosine, 100% of all guanosine nucleotides are 2′-deoxy guanosine, 100% of all cytidine nucleotides are 2′-O-methyl cytidine and 100% of all uridine nucleotides are 2′-O-methyl uridine.

Under rGmH transcription conditions, the transcription reaction mixture comprises 2′-OH guanosine triphosphates, 2′-O-methyl cytidine triphosphates, 2′-O-methyl uridine triphosphates and 2′-O-methyl adenosine triphosphates. The modified oligonucleotides produced using the rGmH transcription mixtures of the present invention comprise substantially all 2′-OH guanosine, 2′-O-methyl cytidine, 2′-O-methyl uridine and 2′-O-methyl adenosine. In a preferred embodiment, the resulting modified oligonucleotides comprise a sequence where at least 80% of all guanosine nucleotides are 2′-OH guanosine, at least 80% of all cytidine nucleotides are 2′-O-methyl cytidine, at least 80% of all uridine nucleotides are 2′-O-methyl uridine and at least 80% of all adenosine nucleotides are 2′-O-methyl adenosine. In a more preferred embodiment, the resulting modified oligonucleotides comprise a sequence where at least 90% of all guanosine nucleotides are 2′-OH guanosine, at least 90% of all cytidine nucleotides are 2′-O-methyl cytidine, at least 90% of all uridine nucleotides are 2′-O-methyl uridine and at least 90% of all adenosine nucleotides are 2′-O-methyl adenosine. In a most preferred embodiment, the resulting modified oligonucleotides comprise a sequence where 100% of all guanosine nucleotides are 2′-OH guanosine, 100% of all cytidine nucleotides are 2′-O-methyl cytidine, 100% of all uridine nucleotides are 2′-O-methyl uridine and 100% of all adenosine nucleotides are 2′-O-methyl adenosine.

Under r/mGmH transcription conditions, the transcription reaction mixture comprises 2′-O-methyl adenosine triphosphate, 2′-O-methyl cytidine triphosphate, 2′-O-methyl guanosine triphosphate, 2′-O-methyl uridine triphosphate and 2′-OH guanosine triphosphate. The resulting modified oligonucleotides produced using the r/mGmH transcription mixtures of the present invention comprise substantially all 2′-O-methyl adenosine, 2′-O-methyl cytidine, 2′-O-methyl guanosine and 2′-O-methyl uridine, wherein the population of guanosine nucleotides has a maximum of about 10% 2′-OH guanosine. In a preferred embodiment, the resulting r/mGmH modified oligonucleotides of the present invention comprise a sequence where at least 80% of all adenosine nucleotides are 2′-O-methyl adenosine, at least 80% of all cytidine nucleotides are 2′-O-methyl cytidine, at least 80% of all guanosine nucleotides are 2′-O-methyl guanosine, at least 80% of all uridine nucleotides are 2′-O-methyl uridine and no more than about 10% of all guanosine nucleotides are 2′-OH guanosine. In a more preferred embodiment, the resulting modified oligonucleotides comprise a sequence where at least 90% of all adenosine nucleotides are 2′-O-methyl adenosine, at least 90% of all cytidine nucleotides are 2′-O-methyl cytidine, at least 90% of all guanosine nucleotides are 2′-O-methyl guanosine, at least 90% of all uridine nucleotides are 2′-O-methyl uridine and no more than about 10% of all guanosine nucleotides are 2′-OH guanosine. In a most preferred embodiment, the resulting modified oligonucleotides comprise a sequence where 100% of all adenosine nucleotides are 2′-O-methyl adenosine, 100% of all cytidine nucleotides are 2′-O-methyl cytidine, 90% of all guanosine nucleotides are 2′-O-methyl guanosine and 100% of all uridine nucleotides are 2′-O-methyl uridine and no more than about 10% of all guanosine nucleotides are 2′-OH guanosine.

Under mRmY transcription conditions, the transcription mixture comprises only 2′-O-methyl adenosine triphosphate, 2′-O-methyl cytidine triphosphate, 2′-O-methyl guanosine triphosphate, 2′-O-methyl uridine triphosphate. The resulting modified oligonucleotides produced using the mRmY transcription mixture of the present invention comprise a sequence where 100% of all adenosine nucleotides are 2′-O-methyl adenosine, 100% of all cytidine nucleotides are 2′-O-methyl cytidine, 100% of all guanosine nucleotides are 2′-O-methyl guanosine and 100% of all uridine nucleotides are 2′-O-methyl uridine.

Under fGmH transcription conditions, the transcription reaction mixture comprises 2′-O-methyl adenosine triphosphates, 2′-O-methyl uridine triphosphates, 2′-O-methyl cytidine triphosphates and 2′-F guanosine triphosphates. The modified oligonucleotides produced using the fGmH transcription conditions of the present invention comprise substantially all 2′-O-methyl adenosine, 2′-O-methyl uridine, 2′-O-methyl cytidine and 2′-F guanosine. In a preferred embodiment, the resulting modified oligonucleotides comprise a sequence where at least 80% of all adenosine nucleotides are 2′-O-methyl adenosine, at least 80% of all uridine nucleotides are 2′-O-methyl uridine, at least 80% of all cytidine nucleotides are 2′-O-methyl cytidine and at least 80% of all guanosine nucleotides are 2′-F guanosine. In a more preferred embodiment, the resulting modified oligonucleotides comprise a sequence where at least 90% of all adenosine nucleotides are 2′-O-methyl adenosine, at least 90% of all uridine nucleotides are 2′-O-methyl uridine, at least 90% of all cytidine nucleotides are 2′-O-methyl cytidine and at least 90% of all guanosine nucleotides are 2′-F guanosine. In a most preferred embodiment, the resulting modified oligonucleotides comprise a sequence where 100% of all adenosine nucleotides are 2′-O-methyl adenosine, 100% of all uridine nucleotides are 2′-O-methyl uridine, 100% of all cytidine nucleotides are 2′-O-methyl cytidine and 100% of all guanosine nucleotides are 2′-F guanosine.

Under dAmB transcription conditions, phosphates, 2′-O-methyl cytidine triphosphates, 2′-O-methyl guanosine triphosphates and 2′-O-methyl uridine triphosphates. The modified oligonucleotides produced using the dAmB transcription mixtures of the present invention comprise substantially all 2′-deoxy adenosine, 2′-O-methyl cytidine, 2′-O-methyl guanosine and 2′-O-methyl uridine. In a preferred embodiment, the resulting modified oligonucleotides comprise a sequence where at least 80% of all adenosine nucleotides are 2′-deoxy adenosine, at least 80% of all cytidine nucleotides are 2′-O-methyl cytidine, at least 80% of all guanosine nucleotides are 2′-O-methyl guanosine and at least 80% of all uridine nucleotides are 2′-O-methyl uridine. In a more preferred embodiment, the resulting modified oligonucleotides comprise a sequence where at least 90% of all adenosine nucleotides are 2′-deoxy adenosine, at least 90% of all cytidine nucleotides are 2′-O-methyl cytidine, at least 90% of all guanosine nucleotides are 2′-O-methyl guanosine and at least 90% of all uridine nucleotides are 2′-O-methyl uridine. In a most preferred embodiment, the resulting modified oligonucleotides of the present invention comprise a sequence where 100% of all adenosine nucleotides are 2′-deoxy adenosine, 100% of all cytidine nucleotides are 2′-O-methyl cytidine, 100% of all guanosine nucleotides are 2′-O-methyl guanosine and 100% of all uridine nucleotides are 2′-O-methyl uridine.

In each case, the transcription products can then be used as the library for input into the in vivo SELEX process to identify oligonucleotides, preferably aptamers, and/or to determine a conserved motif or sequence that has binding specificity to a given target. The resulting sequences are already stabilized, eliminating this step from the post-in vivo SELEX modification process to arrive at an optimized aptamer sequence and giving a more highly stabilized aptamer as a result. Another advantage of the 2′-OMe SELEX process is that the resulting sequences are likely to have fewer 2′-OH nucleotides required in the sequence, possibly none. To the extent 2′OH nucleotides remain they may be removed by performing post-in vivo SELEX modifications.

As described below, lower but still useful yields of transcripts fully incorporating 2′ substituted nucleotides can be obtained under conditions other than the optimized conditions described above. For example, variations to the above transcription conditions include:

The HEPES buffer concentration can range from 0 to 1 M. The present invention also contemplates the use of other buffering agents having a pKa between 5 and 10 including, for example, Tris-hydroxymethyl-aminomethane.

The DTT concentration can range from 0 to 400 mM. The methods of the present invention also provide for the use of other reducing agents including, for example, mercaptoethanol.

The spermidine and/or spermine concentration can range from 0 to 20 mM.

The PEG-8000 concentration can range from 0 to 50% (w/v). The methods of the present invention also provide for the use of other hydrophilic polymer including, for example, other molecular weight PEG or other polyalkylene glycols.

The Triton X-100 concentration can range from 0 to 0.1% (w/v). The methods of the present invention also provide for the use of other non-ionic detergents including, for example, other detergents, including other Triton-X detergents.

The MgCl₂ concentration can range from 0.5 mM to 50 mM. The MnCl₂ concentration can range from 0.15 mM to 15 mM. Both MgCl₂ and MnCl₂ must be present within the ranges described and in a preferred embodiment are present in about a 10 to about 3 ratio of MgCl₂:MnCl₂, preferably, the ratio is about 3-5:1, more preferably, the ratio is about 3-4:1.

The 2′-OMe NTP concentration (each NTP) can range from 5 μM to 5 mM.

The 2′-OH GTP concentration can range from 0 μM to 300 μM.

The 2′-OH GMP concentration can range from 0 to 5 mM.

The pH can range from pH 6 to pH 9. The methods of the present invention can be practiced within the pH range of activity of most polymerases that incorporate modified nucleotides. In addition, the methods of the present invention provide for the optional use of chelating agents in the transcription reaction condition including, for example, EDTA, EGTA and DTT.

### Methods for Selecting Oligonucleotides

### Preferably Aptamers, that Persist in Biological Compartments

Embodiments of the present invention describe methods for selecting oligonucleotides, preferably aptamers, that persist for a given period of time in biological compartments. In some embodiments, the biological compartment is within a multicellular organism, for example, a living multicellular organism. In one embodiment, the multicellular organism is a mammal.

In some embodiments, it is contemplated that the methods for selecting oligonucleotides, preferably aptamers, that persist in a specific organ comprises the steps of: i) preparing a library of oligonucleotides ii) introducing the library of oligonucleotides into a biological compartment of a living organism, iii) waiting for a period of time to elapse, iv) harvesting the biological compartment (or portion thereof), v) collecting oligonucleotides, of the library of oligonucleotides, from the biological compartment and v) amplifying the oligonucleotides contained therein. In one embodiment, this process is repeated iteratively until an enriched population of oligonucleotides, preferably aptamers, is identified. In one embodiment, the iterative repetitions of in vivo SELEX is in a range of three to nine rounds.

In some embodiments, it is contemplated that the methods for selecting oligonucleotides, preferably aptamers, that persist in a specific organ comprises the steps of: i) preparing a library of oligonucleotides ii) introducing the library of oligonucleotides into an artery that perfuses an organ, iii) waiting for a period of time to elapse, iv) harvesting the oligonucleotide perfused organ (or a portion thereof), v) collecting oligonucleotides, of the library of oligonucleotides, from the oligonucleotide perfused organ and v) amplifying the oligonucleotides contained therein. In one embodiment, the organ is selected from the group consisting of kidney, liver and spleen. In one embodiment, this process is repeated iteratively until an enriched population of oligonucleotides, preferably aptamers, is identified. In one embodiment, the iterative repetitions of in vivo SELEX is in a range of three to nine rounds.

It is not intended that the present invention be limited by the elapsed time in step iii). The elapsed time in step iii) may be regulated so as to vary population of oligonucleotides, preferably aptamers, that are collected in step v). In one embodiment, the organ (or portion thereof) is harvested within five minutes to two hours after the organ is perfused with a library of oligonucleotides. This period of elapsed time would allow the organ to be completely perfused by the library of oligonucleotides but would minimize the exposure of the same to the metabolic processes within the organ. In one embodiment, the organ (or portion thereof) is harvested within 24 hrs to 472 hrs after the organ is perfused with a library of oligonucleotides. This period of elapsed time would further select for oligonucleotides, preferably aptamers, that are: i) have a higher affinity for the organ, ii) resistant to metabolic processes within the organ and iii) in some embodiment may select for oligonucleotides, preferably aptamers, that are sequestered within the cytoplasm of cells within the organ.

In some embodiments, it is contemplated that the method for selecting oligonucleotides that persist in a specific organ comprises the steps of: i) preparing a library of oligonucleotides, ii) introducing the library of oligonucleotides into an artery that perfuses an organ, iii) waiting for a period of time to elapse, iv) collecting oligonucleotides of the library of oligonucleotides from a sample of blood collected from at least one vein that drains the organ and v) amplifying the oligonucleotides, preferably aptamers, contained therein. In one embodiment, the organ is the kidney, the artery is the renal artery and the vein is the renal vein. In one embodiment, this process is repeated iteratively until an enriched population of oligonucleotides, preferably aptamers, is identified. In one embodiment, the iterative repetitions of in vivo SELEX is in a range of three to nine rounds.

It is not intended that the present invention be limited by the elapsed time in step iii). The elapsed time in step iii) may be regulated so as to vary population of oligonucleotides, preferably aptamers, that are collected in step iv). In one embodiment, the blood is sampled within five minutes to two hours after the organ is perfused with a library of oligonucleotides. This period of elapsed time would allow the organ to be completely perfused by the library of oligonucleotides and any oligonucleotides, preferably aptamers, collected in the blood would be characterized by a short residence time in the organ. In one embodiment, the blood is sampled within 24 hrs to 472 hrs after the organ is perfused with a library of oligonucleotides. This period of elapsed time would further select for oligonucleotides, preferably aptamers, that have resided in the organ for at least 24 hrs to 472 hrs, resisted the metabolic processes within the organ for that same period of time, and have subsequently been elaborated by the organ. In this respect oligonucleotides, preferably aptamers, selected in this manner would be characterized by a defined residence time. If such an aptamer was conjugated with another therapeutic moiety (e.g., aptamer, small molecule, or protein) that targeted tissues or cells within the organ it is contemplated the in vivo SELEX aptamer would provide means to chaperon the therapeutic moiety for a know amount of time. Such regulation could maximize the therapeutic effect of the therapeutic moiety and minimize its toxicity.

In some embodiments, the biological compartment is a tissue. While it is not intended that the methods of the present invention be limited to any specific tissue, in some embodiments the tissue is selected from the group consisting of: epithelial tissue, connective tissue, muscle tissue, nervous tissue. In a preferred embodiment the tissue is blood.

In some embodiments, it is contemplated the tissue is tumor tissue. In some embodiments, the tumor tissue is benign. In some embodiments, the tumor is malignant.

In some embodiments, it is contemplated that the in vivo SELEX methods of the present invention could select for oligonucleotides, preferably aptamers, with varying residence times within a tumor. It is contemplated that this functionality would allow for the tunability of the presentation of a therapeutic moiety (e.g., aptamer, small molecule, or protein) that could be conjugated with the in vivo SELEX selected aptamer. That is to say, highly cytotoxic therapeutic moieties could be conjugated to oligonucleotides, preferably aptamers, with a relatively short residence time while less cytotoxic could be conjugated to oligonucleotides, preferably aptamers, with a longer residence time, thereby, maximizing the therapeutic effect of a given therapeutic moiety.

In some embodiments, the method for selecting oligonucleotides, preferably aptamers, that persist in a biological compartment comprises the steps of: i) preparing a library of oligonucleotides ii) introducing the library of oligonucleotides into the circulatory system of a mammal, iii) waiting for a period of time to elapse sufficient to allow the oligonucleotides to distribute throughout the circulatory system, iv) collecting oligonucleotides of the library of oligonucleotides from a sample of blood collected from the mammal and v) amplifying the oligonucleotides, preferably aptamers, contained therein. In some embodiments, this process is repeated iteratively until an enriched population of oligonucleotides, preferably aptamers, is identified. For clarity, an enriched population of oligonucleotides, preferably aptamers, is characterized by individual species of oligonucleotides, preferably aptamers, that persist in a biological compartment (the blood, in this embodiment) as compared to the unselected oligonucleotide library. In one embodiment, the iterative repetitions of in vivo SELEX is in a range of three to nine rounds. In some embodiments, the library of oligonucleotides is nuclease-stabilized. In one embodiment, the nuclease-stabilized library is MNA. In one embodiment, the elapsed time, in step iii), is in a range from 5 mins. to 300 hrs. In one embodiment, the elapsed time, in step ii), is in a range from 2 hrs. to 300 hrs. In one embodiment, the elapsed time, in step ii), is in a range from 64 hrs. to 300 hrs. In one embodiment, the elapsed time, in step ii), is in a range from 128 hrs. to 256 hrs. By increasing the elapsed time in step iii) the resulting oligonucleotide collected in step iv) will have greater: i) nuclease resistance and ii) resistance to renal clearance.

It is not intended that the present invention be limited to the introduction of a single oligonucleotide library having a single pool composition. That is to say, it is contemplated that multiple pool libraries (e.g., DNA, RNA, MNA, rRfY, mRfY, rRmY, dRmY, rGmH, mRmY, fRmH and/or dAmB) may be introduced into a biological compartment simultaneously and subsequently collected from tissues and differentially amplified. In some embodiments, multiple biological compartments could be sampled simultaneously. In some embodiments, multiple biological compartments could be sampled sequentially.

Embodiments of the present invention describe methods, i.e., in vivo SELEX, for selecting oligonucleotides, preferably aptamers, that persist for an extended period of time in a biological compartment. In some embodiments, oligonucleotides, preferably aptamers, selected via in vivo SELEX offer an alternative to pegylation as a means for extending the pharmacokinetic (PK) half-life of oligonucleotides, preferably aptamers, in a biological compartment.

In one embodiment, persistence in the blood was achieved by intravenously dosing a library of nuclease-resistant fully 2′-OMe RNA transcripts to a into a live mouse. After a proscribed time interval had elapsed the blood was collected and the remaining transcript sequences were amplified using RT-PCR. An enriched library was then generated via transcription with 2′-OMe NTPs for the next round of in vivo SELEX. The proscribed time interval was increased from over nine successive rounds of in vivo SELEX, at which point individual transcripts were sequenced and characterized. This in vivo SELEX process identified two distinct sequence motifs (CLN9397 and CLN 9406) that persisted in the mouse circulatory compartment with a monophasic PK half-life of approximately 15 hours.

This is an approximately 200-fold improvement over sequences randomly selected from the initial library and is similar to half lives observed for 40 kDa PEG-aptamer conjugates which generally have half-lives in the 5-30 hour range. In some embodiments, it is contemplated that an unpegylated aptamer demonstrating such persistence in the blood could be conjugated together with another aptamer binding to a therapeutic target protein, thereby, forming a bivalent aptamer.

### Aptamer Medicinal Chemistry

Once oligonucleotides, preferably aptamers, that bind to a desired target are identified, several techniques may be optionally performed to further increase the functional characteristics of the identified aptamer sequences. Oligonucleotides, preferably aptamers, that bind to a desired target identified through the in vivo SELEX process may be optionally truncated to obtain the minimal aptamer sequence (also referred to herein as “minimized construct”) having the desired functional characteristics. One method of accomplishing this is by using folding programs and sequence analysis (e.g., aligning clone sequences resulting from a selection to look for conserved motifs and/or covariation) to inform the design of minimized constructs. Biochemical probing experiments can also be performed to determine the 5′ and 3′ boundaries of an aptamer sequence to inform the design of minimized constructs. Minimized constructs can then be chemically synthesized and tested for functional characteristics as compared to the non-minimized sequence from which they were derived. Variants of an aptamer sequence containing a series of 5′, 3′ and/or internal deletions may also be directly chemically synthesized and tested for functional characteristics as compared to the non-minimized aptamer sequence from which they were derived.

The kinds of substituent that can be utilized by the Aptamer Medicinal Chemistry process are only limited by the ability to generate them as solid-phase synthesis reagents and introduce them into an oligomer synthesis scheme. The process is certainly not limited to nucleotides alone. Aptamer Medicinal Chemistry schemes may include substituents that introduce steric bulk, hydrophobicity, hydrophilicity, lipophilicity, lipophobicity, positive charge, negative charge, neutral charge, zwitterions, polarizability, nuclease-resistance, conformational rigidity, conformational flexibility, protein-binding characteristics, mass etc. Aptamer Medicinal Chemistry schemes may include base-modifications, sugar-modifications or phosphodiester linkage-modifications.

Target binding affinity of the oligonucleotides, preferably aptamers, of the present invention can be assessed through a series of binding reactions between the aptamer and target (e.g., a protein) in which trace ³²P-labeled aptamer is incubated with a dilution series of the target in a buffered medium then analyzed by nitrocellulose filtration using a vacuum filtration manifold. Referred to herein as the dot blot binding assay, this method uses a three layer filtration medium consisting (from top to bottom) of nitrocellulose, nylon filter and gel blot paper. RNA that is bound to the target is captured on the nitrocellulose filter whereas the non-target bound RNA is captured on the nylon filter. The gel blot paper is included as a supporting medium for the other filters. Following filtration, the filter layers are separated, dried and exposed on a phosphor screen and quantified using a phosphorimaging system from which. The quantified results can be used to generate aptamer binding curves from which dissociation constants (K_{D}) can be calculated. In a preferred embodiment, the buffered medium used to perform the binding reactions is 1× Dulbecco's PBS (with Ca⁺⁺ and Mg⁺⁺) plus 0.1 mg/mL BSA.

In addition to describing methods (i.e., in vivo SELEX for the discovery of oligonucleotide sequence motifs that have a propensity to be targeted to, or persist in, biological compartments; embodiments of the present invention also contemplate therapeutic or diagnostic compositions that are linked to the sequence motifs, including those comprised of nucleotides and including contiguous oligonucleotides having both functions.

The oligonucleotides, preferably aptamers, of the present invention may be routinely adapted for diagnostic purposes according to any number of techniques employed by those skilled in the art. Diagnostic utilization may include both in vivo or in vitro diagnostic applications. Diagnostic agents need only be able to allow the user to identify the presence of a given target at a particular locale or concentration. Simply the ability to form binding pairs with the target may be sufficient to trigger a positive signal for diagnostic purposes. Those skilled in the art would also be able to adapt any oligonucleotide, preferably aptamer, by procedures known in the art to incorporate a labeling tag in order to track the presence of such oligonucleotide, preferably aptamer. Such a tag could be used in a number of diagnostic procedures.

### Modulation of Pharmacokinetics and Biodistribution of Aptamer Therapeutics

The tunability of (i.e., the ability to modulate) aptamer pharmacokinetics is further facilitated through conjugation of modifying moieties (e.g., PEG polymers) to the aptamer and/or the incorporation of modified nucleotides (e.g., 2′-fluoro or 2′-O-methyl) to alter the chemical composition of the oligonucleotide, preferably aptamer. The ability to tune oligonucleotide, preferably aptamer, pharmacokinetics is used in the improvement of existing therapeutic applications, or alternatively, in the development of new therapeutic applications. For example, in some therapeutic applications, e.g., in anti-neoplastic or acute care settings where rapid drug clearance or turn-off may be desired, it is desirable to decrease the residence times of oligonucleotides, preferably aptamers, in the circulation.

It is contemplated the oligonucleotides, preferably aptamers, selected for their persistence in a biological compartment, as described by the in vivo SELEX methods of the present invention, may be further modulated by conjugating an aptamer to a modulating moiety such as a small molecule, peptide, or polymer terminal group, or by incorporating modified nucleotides into an aptamer. Oligonucleotides, preferably aptamers, can be conjugated to a variety of modifying moieties, such as high molecular weight polymers, e.g., PEG; peptides, e.g., Tat (a 13-amino acid fragment of the HIV Tat protein (Vives, et al. (1997), J. Biol. Chem. 272 (25): 16010-7)), Ant (a 16-amino acid sequence derived from the third helix of the Drosophila antennapedia homeotic protein (Pietersz, et al. (2001), Vaccine 19 (11-12): 1397-405)) and Arg₇ (a short, positively charged cell-permeating peptides composed of polyarginine (Arg₇) (Rothbard, et al. (2000), Nat. Med. 6 (11): 1253-7; Rothbard, J et al. (2002), J. Med. Chem. 45 (17): 3612-8)); and small molecules, e.g., lipophilic compounds such as cholesterol.

While embodiments of the present invention describe in vivo methods for the preferential selection of oligonucleotides, preferably aptamers, that persist in a biological compartment, it is contemplated that subsequent conjugation with, in one example, PEG groups may further extend the residence time of these same in vivo selected oligonucleotides, preferably aptamers, in a biological system. For example, described in the non-provisional application referenced above (U.S. Ser. No. 11/075,648 filed on Mar. 7, 2005 and entitled “Controlled Modulation of the Pharmacokinetics and Biodistribution of Aptamer Therapeutics”), conjugation of an aptamer therapeutic with a 20 kDa PEG polymer hinders renal filtration and promotes aptamer distribution to both healthy and inflamed tissues. Furthermore, the 20 kDa PEG polymer-aptamer conjugate proves nearly as effective as a 40 kDa PEG polymer in preventing renal filtration of oligonucleotides which, in a preferred embodiment, include aptamers. While one effect of PEGylation is on aptamer clearance, the prolonged systemic exposure afforded by presence of the 20 kDa moiety also facilitates distribution of aptamer to tissues, particularly those of highly perfused organs and those at the site of inflammation.

All publications and patent documents cited herein are incorporated herein by reference as if each such publication or document was specifically and individually indicated to be incorporated herein by reference. Citation of publications and patent documents is not intended as an admission that any is pertinent prior art, nor does it constitute any admission as to the contents or date of the same. The invention having now been described by way of written description, those of skill in the art will recognize that the invention can be practiced in a variety of embodiments and that the foregoing description and examples below are for purposes of illustration and not limitation of the claims that follow.

### EXAMPLES

### Example 1

### In Vivo SELEX for Blood Persistence in the Mouse

### Library Generation

A library of DNA transcription templates was prepared by PCR amplification of ARC3428 [GGGAGACAAGAATAAAGCGAGTTNNNNNNNNNNNNNNNN NNNNNNNNNNNNNNAAGAGTCGATGATGCTTAGCTAG] (SEQ ID NO: 4) with 5′ primer KMT226.120.A [GATCGATCGATCGATCGATCTAATACGACTCACTATA GGGAGACAAGAAT] (SEQ ID NO: 5) and 3′ primer KMT226.120.B [CTAGCTAAGCATCATCGACTCTT] (SEQ ID NO: 6) each at 500 nM and Taq polymerase (NEB, Beverly Mass.) according to manufacturer's instructions with cycling at 94° C. for 0.5 min., 65° C. for 1 min. and 72° C. for 3 min. The resultant library of transcription templates was then used to program a 10 ml MNA transcription by incubating it under standard MNA transcription conditions as follows: 200 mM HEPES, 40 mM DTT, 2 mM spermidine, 0.01% TritonX-100, 10% PEG-8000, 8 mM MgCl₂, 2.5 mM MnCl₂, 1.5 mM 2′-OMe CTP, 1.5 mM 2′-OMe UTP, 1.5 mM 2′-OMe GTP, 1.5 mM 2′-OMe ATP, 1 mM GMP, pH 7.5, 0.01 units/μl inorganic pyrophosphatase, 2 μg/ml mutant T7 RNA polymerase (Y639L/H784A/K378R) and 50 nM template DNA at 37° C. overnight. The resultant transcripts were purified using denaturing PAGE and then extracted from excised gel slices by electroelution followed by ethanol precipitation.

Selection and Amplification Protocol

For the initial SELEX round, the library of MNA transcripts was prepared as a 5 uM solution in 0.9% saline, filtered through a 200 nm filter and 200 ul was dosed intravenously via the lateral tail vein to each of two CD-1 mice. 3×10¹⁴ unique MNA transcripts were dosed to each mouse with an average copy number of two of each transcript in each mouse. After two hrs. the mice were sacrificed, blood samples were collected via cardiac puncture and samples were placed into tubes coated with the lithium salt of heparin.

To each 1000 ul of whole blood were added 10 ul of 10 mg/ml sheared salmon sperm DNA, 10 ul of 10 mg/ml yeast tRNA, 100 ul of 10× Proteinase K buffer and 60 units of Proteinase K (at 20 mg/ml in 20 mM Tris, 1 mM CaCl₂, 50% glycerol, pH 7.4). 10× Proteinase K buffer is 100 mM Tris, 150 mM EDTA, 1% SDS, pH 8.0. This mixture was then incubated at 55° C. for one hour and then at 90° C. for 10 min. followed by centrifugation at 16,100 rcf for 5 min., transferred to a new tube and heated at 90° C. for 10 min., centrifugation at 16,100 rcf for 15 min. and subsequently passaged through a NAP-10 column (Pharmacia) that had been pre-equilibrated with deionized water.

This sample was diluted a further five-fold into a reverse transcription mixture using Thermoscript Reverse Transcriptase (Invitrogen, Carlsbad, Calif.) according to manufacturers directions at 65° C. The resulting incubated mixture was then diluted four-fold into a PCR mixture and amplified with 5′ primer KMT226.120.A and 3′ primer KMT226.120.B each at 500 nM and Taq polymerase (NEB, Beverly Mass.) according to manufacturer's instructions with cycling at 94° C. for 0.5 min., 65° C. for 1 min. and 72° C. for 3 min. The corresponding enriched library of transcription templates was then used to program an in vitro transcription to generate an enriched library of MNA transcripts that were used for the succeeding selection step.

In each round, two mice were dosed and their MNA transcripts were amplified by RT-PCR before equal amounts of transcription template were combined in a single MNA transcription mixture to generate transcripts for dosing in the subsequent selection step. Subsequent to the first round, rounds of SELEX were undertaken as described for the initial SELEX round except for the following differences, which are in Table 1: the transcription volume was reduced in some rounds, the time allowed to elapse between dosing to and the sacrifice of the mice was approximately doubled for each succeeding round of selection, the volume of blood utilized for amplification was reduced, the RT dilution factor and volume was reduced, the PCR dilution factor and volume was reduced, the concentration of MNA in the dosing solution was reduced in some rounds.

TABLE 1

| | | | | | | | | | | Tran- | | |
|---|---|---|---|---|---|---|---|---|---|---|---|---|
| | Input | Time in | | Blood | RT | RT | PCR | PCR | PCR | scription | | Sequence |
| Round | MNA | mouse | | used | dilution | volume | dilution | volume | cycles | volume | | Data |
| 0 | — | — | | — | — | — | — | — | — | 10 | ml | Y |
| 1 | 2.0 nmol | 2 | h | 100% | 5-fold | 18.5 ml | 4-fold | 74 ml | 25 | 10 | ml | N |
| 2 | 2.0 nmol | 5 | h | 50% | 5-fold | 7.5 ml | 4-fold | 30 ml | 21 | 10 | ml | N |
| 3 | 2.0 nmol | 8 | h | 12% | 10-fold | 7.5 ml | 4-fold | 30 ml | 15 | 10 | ml | N |
| 4 | 2.0 nmol | 16 | h | 6% | 10-fold | 1.8 ml | 4-fold | 7.2 ml | 20 | 5 | ml | Y |
| 5 | 1.2 nmol | 32 | h | 6% | 10-fold | 1.5 ml | 4-fold | 6.0 ml | 23 | 5 | ml | N |
| 6 | 1.6 nmol | 64 | h | 6% | 10-fold | 1.8 ml | 4-fold | 7.2 ml | 25 | 5 | ml | Y |
| 7 | 2.0 nmol | 136 | h | 6% | 10-fold | 1.8 ml | 4-fold | 7.2 ml | 22 | 10 | ml | N |
| 8 | 2.0 nmol | 472 | h | 6% | 10-fold | 1.8 ml | 4-fold | 7.2 ml | 27 | 10 | ml | Y |
| 9 | 1.2 nmol | 1000 | h | 6% | 10-fold | 1.4 ml | 4-fold | 5.6 ml | 27 | 5 | ml | Y |

Sequence composition and frequency data are presented in Table 2

TABLE 2

| | Total | Unique | Nucleotide frequency in | | | |
|---|---|---|---|---|---|---|
| | clones | sequences | degnerate region (%) | | | |
| Round | sequenced | observed | A | G | C | U |
| 0 | 40 | 40 | 31.4 | 19.8 | 20.6 | 28.3 |
| 4 | 47 | 47 | 30.3 | 12.6 | 27.4 | 29.7 |
| 6 | 65 | 57 | 30.2 | 8.5 | 30.5 | 30.8 |
| 8 | 79 | 70 | 31.0 | 9.9 | 29.4 | 29.7 |
| 9 | 75 | 38 | 30.0 | 11.7 | 25.7 | 32.7 |

### Example 2

### Library Persistence Assays

In order to characterize the libraries selected in Example 1, a preliminary pharmacokinetic evaluation was undertaken in which equal amounts of the (unselected) library and the selected library after eight rounds of SELEX were each dosed intravenously via the lateral tail vein to six mice. Pairs of mice for each library were then sacrificed at 5 mins., 2 hrs. and 64 hrs., blood samples were collected via cardiac puncture and samples were placed in tubes coated with the lithium salt of heparin. Equal volumes of blood were then processed with proteinase K as described in Example 1, the resultant MNA transcripts were then amplified by RT-PCR, and the number of PCR cycles required to reach an arbitrary amplicon concentration was recorded and compared for the two libraries. The results of this experiment are shown in Table 3.

TABLE 3

| | | |
|---|---|---|
| | | Number of PCR cycles to |
| | | achieve arbitrary |
| Round Number | Elapsed time | amplicon concentration |

| | | | |
|---|---|---|---|
| 0 | 5 | mins. | 10 |
| 8 | 5 | mins. | 10 |
| 0 | 2 | hrs. | 22 |
| 8 | 2 | hrs. | 11 |
| 0 | 64 | hrs. | >25 |
| 8 | 64 | hrs. | 15 |

Assuming a PCR efficiency of 1.8 (that the concentration of amplicon increases by a factor of 1.8 in each cycle of PCR), then between the 5 minute and 2 hour time points the (unselected, round 0) library has an average half-life of about 10 minutes in these mice and the selected (round 8) library has an average half-life of about 2 hrs. Between the 2 hr. and 64 hr. time points, the selected (round 8) library has an average half-life of about 20 hr. in these mice.

### Example 3

### Library Location Assays

100 ul blood samples from the 64 hour time point of the selected (round 8) library as described in Example 2 were centrifuged at 1000 rcf for 10 minutes in order to pellet the cells. The supernatant was removed and placed in a new tube and centrifuged at 1000 rcf for 10 minutes a second time before being removed and placed in a new tube and centrifuged at 1000 rcf for 10 minutes a third time and was then placed in a fresh tube. The cell pellet was resuspended in 1×PBS and then centrifuged a second time before the removal of the supernatant and resuspension in 1×PBS and then centrifuged a third time before the removal of the supernatant and a final resuspension in 1×PBS. Each sample was then separately processed with proteinase K as described in Example 1 and the resultant MNA transcripts were then amplified by RT-PCR and the number of PCR cycles required to reach an arbitrary amplicon concentration was recorded and compared for the two libraries. This experiment showed that the round 8 MNA transcript library is found in the supernatant and not the cell pellet by a factor of at least eight PCR cycles (17 versus >25) and so, assuming a PCR efficiency of 1.8, this corresponded to a difference factor of greater than one hundred. While it is not intended the present invention be limited to any specific mechanism, the hypothesis that the extended persistence of the round 8 selected library is not dependent upon binding to a cell-surface target is consistent with these data.

### Example 4

### Cassette Pharmacokinetic Study Of Selected Clones Discovered In Example 1

The sequences for all clones referenced in the Experimental section are set out in Table 5. Eight DNA clones from the selected library (rounds 6 and 8) (encoding CLN9397, CLN9406, CLN10217, CLN10218, CLN10231, CLN10235, CLN10237, CLN10238 and CLN10268) and 3 from the (unselected) library (encoding CLN9908, CLN9913 and CLN9917) were separately amplified by PCR and then transcribed into MNA transcripts separately as described in Example 1 (all clones described in the Experimental section are set out in Table 5). Equal amounts of these 11 different transcripts were then mixed to give a dosing solution that contained 173 nM of each individual clone in 0.9% saline. 200 ul of this solution was dosed to each CD-1 mouse intravenously via the lateral tail vein. Mice were sacrificed over a time-course, blood samples were taken by cardiac puncture and placed into tubes coated with the lithium salt of heparin and were investigated using two different methods as described below.

Universal Analysis

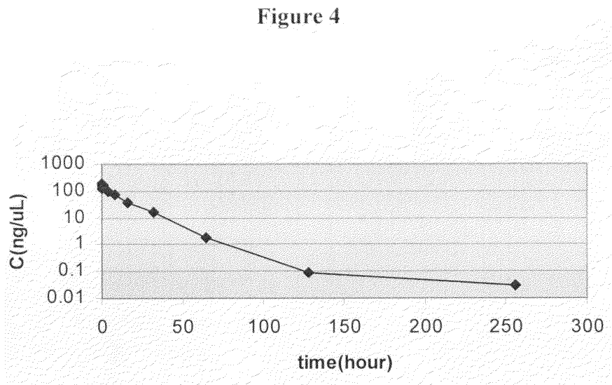

Blood from each time-point was processed with proteinase K as described in Example 1, amplified by RT-PCR and the number of cycles of PCR that were required to reach an arbitrary amplicon concentration were recorded. The number of cycles of PCR was then used to calculate the original MNA concentration in the blood samples at each time point. These data are shown in FIG. 4.

The universal analysis shows the average pharmacokinetic behavior of this ensemble of eleven clones, three from the (unselected) library and eight from the selected (rounds 6 and 8) library. The average pharmacokinetic half-lives for this ensemble are 4 hrs. (for the first 4 hrs.), 12 hours (for the 124 hrs. from 4 hrs. to 128 hrs.) and 90 hrs. (for the 128 hrs. from 128 hrs. to 256 hrs.). As further illustrated by FIG. 4, the starting concentration is 200 ng/ul which represents all 11 clones and therefore is 18 ng/ul for each clone. At 128 hrs. the concentration is 0.1 ng/ul which represents 2 clones and therefore is 0.05 ng/ul for each clone. The ratio of these is (18/0.05)=360.

Specific Analysis

In order to identify the MNA clones with the greatest propensity to persist in the mouse over longer timescales, PCR-amplified material from each of the longest two time-points (128 hrs. and 256 hrs.) were sequenced. These data are presented in Table 4.

TABLE 4

| | SELEX Round | Fraction of | Fraction of |
|---|---|---|---|
| | clone first | sequences | sequences |
| Clone | observed in | at 128 hrs. | at 256 hrs. |
| CLN9397 | 6 | 37% | 22% |
| CLN9406 | 6 | 48% | 45% |
| CLN10217 | 8 | 13% | 8.2% |
| CLN10218 | 8 | 0.6% | 10% |
| CLN10231 | 8 | 0% | 0% |
| CLN10237 | 8 | 0% | 0% |
| CLN10238 | 8 | 0% | 0% |
| CLN10268 | 8 | 0% | 0% |
| CLN9908 | 0 | 0.6% | 0% |
| CLN9913 | 0 | 0% | 0% |
| CLN9917 | 0 | 0% | 14% |

Combining these data with the data generated in the universal analysis demonstrates that, over the 124 hr. period from 4 hrs. to 128 hrs., the average half-lives for persistence in the mouse are 14 hrs. for CLN9397 and 15 hrs. for CLN9406. Clones CLN9397 and CLN9406 have the greatest tendency to persist in the blood over the time-periods evaluated in this experiment selected for further study. See, Table 5 for clone sequences.

### Example 5

### Identification Of Binding Partners of CLN9397 and CLN9406

Qualification of Synthetic Clones for Further Study

3′-idT, 5′-biotinylated MNA oligonucleotides with sequences corresponding to CLN9397 and CLN9406 were synthesized, ARC4943 [Biotin-mGmGmGmAmGmAm CmAmAmGmAmAmUmAm AmAmG mCmGmAmGmUmUmUmGm UmAmUmUm CmUmUmUmCmGm AmAmCmUmUmUmCmUmAmAmCmAmCmAmUmCmAmCmAmAm GmAmGmUmCmGmAmUmGmAmUmGmCmUmUmAmGmCmUmAmG-idT] (SEQ ID NO: 7) and ARC4944 [Biotin-mGmGmGmAmGmAmCmAmAmGmAmAm UmAmAmAmG mCmGmAmGmUmUmUmGmCmUmAmUmGmUmCmAmUmUmCm UmAmGmUmUmCmAmCmAmUmCmUmUmCmAmCmAmAmAmGmAmGmUmCmGmAm UmGmAmUmGmCmUmUmAmGmCmUmAmG-idT] (SEQ ID NO: 8) respectively, purified by HPLC and 200 ul of a 5 uM solution of each in 0.9% saline were dosed to each of two CD-1 mice intravenously via the lateral tail vein. After 100 hrs. had elapsed the mice were sacrificed, blood samples were taken by cardiac puncture and samples were placed into tubes coated with the lithium salt of heparin. 100 ul of whole blood was processed with proteinase K, as described in Example 1, diluted 10-fold into a reverse transcription reaction as described in Example 1 and then diluted 10-fold into a PCR as described in Example 1. Analysis of the progress of the PCR showed that the reverse transcriptase-dependent amplicon began to become visible by ethidium-PAGE at 20 cycles for ARC4943 and at 25 cycles and therefore that these constructs are both clearly functional regarding PK-extension.

TABLE 5
Clones By Round Number: 0

| | |
|---|---|
| CLN9931 | GGGAGACAAGAAUAAAGCGAGUUAAGCAGUAGAUUGA |
| SEQ ID NO 9 | UAUCCGAUAGAAAAGUAAGAGUCGAUGAUGCUUAGCU |
| | AGAAGGGCGAAUUCGCGGCCGCUAAAUUCAUUUCGAC |
| | CUCUCUGCUAGC |
| CLN9928 | GGGAGACAAGAAUAAAGCGAGUUAACAAUUUCGACCU |
| SEQ ID NO 10 | CUCUGCUAGC |
| CLN9929 | GGGAGACAAGAAUAAAGCGAGUUCAAACACAGCAUCC |
| SEQ ID NO 11 | GUUUCGACCUCUCUGCUAGC |
| CLN9930 | GGGAGACAAGAAUAAAGCGAGUUACACAGUGCCAGUU |
| SEQ ID NO 12 | UCGACCUCUCUGCUAGC |
| CLN6091 | GGGAGACAAGAAUAAAGCGAGUUUUUUCGACCUCUCU |
| SEQ ID NO 13 | GCUAGC |
| CLN9926 | GGGAGACAAGAAUAAAGCGAGUUUGUUGUUUCGACCU |
| SEQ ID NO 14 | CUCUGCUAGC |
| CLN9927 | GGGAGACAAGAAUAAAGCGAGUUUGGAGUUAGAUUUC |
| SEQ ID NO 15 | GACCUCUCUGCUAGC |
| CLN9923 | GGGAGACAAGAAUAAAGCGAGUUCCACACUAAAUGGA |
| SEQ ID NO 16 | GGGUUGGAAUUAGUCAAGAGUCGAUGAUGCUUAGCUA |
| | GAAGGGCGAAUUCGCGGCCGCUAAAUUCAUUUCGACC |
| | UCUCUGCUAGC |
| CLN9924 | GGGAGACAAGAAUAAAGCGAGUUCGAUCUUCUUUCGA |
| SEQ ID NO 17 | CCUCUCUGCUAGC |
| CLN9925 | GGGAGACAAGAAUAAAGCGAGUUUGCCUUUUCGACCU |
| SEQ ID NO 18 | CUCUGCUAGC |
| CLN9920 | GGGAGACAAGAAUAAAGCGAGUUUAAAAUAGUGAUUU |
| SEQ ID NO 19 | UCGACCUCUCUGCUAGC |
| CLN9921 | GGGAGACAAGAAUAAAGCGAGUUGACAAUCACUUUUC |
| SEQ ID NO 20 | GACCUCUCUGCUAGC |
| CLN9922 | GGGAGACAAGAAUAAAGCGAGUUAGUCCAUUUCGACC |
| SEQ ID NO 21 | UCUCUGCUAGC |
| CLN9917 | GGGAGACAAGAAUAAAGCGAGUUCCGUAGUGCAAGGC |
| SEQ ID NO 22 | UCAAGGUCUACCACUGAAGAGUCGAUGAUGCUUAGCU |
| | AGAAGGGCGAAUUCGCGGCCGCUAAAUUCAUUUCGAC |
| | CUCUCUGCUAGC |
| CLN9918 | GGGAGACAAGAAUAAAGCGAGUUUGCAGAUCUUUCGA |
| SEQ ID NO 23 | CCUCUCUGCUAGC |
| CLN9919 | GGGAGACAAGAAUAAAGCGAGUUUCGUAACUAAUGUA |
| SEQ ID NO 24 | GAUGGUGGAUAAGUUCAAGAGUCGAUGAUGCUUAGCU |
| | AGAAGGGCGAAUUCGCGGCCGCUAAAUUUCGACCUCU |
| | CUGCUAGC |
| CLN9914 | GGGAGACAAGAAUAAAGCGAGUUACGUUUUCGACCUC |
| SEQ ID NO 25 | UCUGCUAGC |
| CLN9915 | GGGAGACAAGAAUAAAGCGAGUUAUCAGCCUAUCCGU |
| SEQ ID NO 26 | UAAAGAUUAUUCAUUGAAGAGUUUCGACCUCUCUGCU |
| | AGC |
| CLN9916 | GGGAGACAAGAAUAAAGCGAGUUCAGUUUCGACCUCU |
| SEQ ID NO 27 | CUGCUAGC |
| CLN9911 | GGGAGACAAGAAUAAAGCGAGUUCUGUACGUUUCGAC |
| SEQ ID NO 28 | CUCUCUGCUAGC |
| CLN9912 | GGGAGACAAGAAUAAAGCGAGUUACGGAGAUUUUCGA |
| SEQ ID NO 29 | CCUCUCUGCUAGC |
| CLN9913 | GGGAGACAAGAAUAAAGCGAGUUCCUAAAAUUCACCG |
| SEQ ID NO 30 | AGAUUUAUCAGGCGUUAAGAGUCGAUGAUGCUUAGCU |
| | AGAAGGGCGAAUUCGCGGCCGCUAAAUUCAUUUCGAC |
| | CUCUCUGCUAGC |
| CLN9944 | GGGAGACAAGAAUAAAGCGAGUUUUUUAAAGAUGUGU |
| SEQ ID NO 31 | UUCGACCUCUCUGCUAGC |
| CLN9909 | GGGAGACAAGAAUAAAGCGAGUUUAAGGUUUACUCCC |
| SEQ ID NO 32 | AUUUCGACCUCUCUGCUAGC |
| CLN9910 | GGGAGACAAGAAUAAAGCGAGUUUGAACCCUUUCGAC |
| SEQ ID NO 33 | CUCUCUGCUAGC |
| CLN9942 | GGGAGACAAGAAUAAAGCGAGUUCACUCCGUUUAAUU |
| SEQ ID NO 34 | UCGACCUCUCUGCUAGC |
| CLN9943 | GGGAGACAAGAAUAAAGCGAGUUAUUUUUCGACCUCU |
| SEQ ID NO 35 | CUGCUAGC |
| CLN833 | GGGAGACAAGAAUAAAGCGAGUUAUUUCGACCUCUCU |
| SEQ ID NO 36 | GCUAGC |
| CLN9939 | GGGAGACAAGAAUAAAGCGAGUUUCUCUGGCCUCGAU |
| SEQ ID NO 37 | UUUGUUACCAUAGCUAAAGAGUCGAUGAUGCUUAGCU |
| | AGAAGGGCGAAUUCGCGGCCGCUAAAUUCAUUUCGAC |
| | CUCUCUGCUAGC |
| CLN9940 | GGGAGACAAGAAUAAAGCGAGUUGACAGCUUUAUUUC |
| SEQ ID NO 38 | AUGCAAAACAAAACGGAAGAGUCGAUGAUGCUUAGCU |
| | AGAAGGGCGAAUUCGCGGCCGCUAAAUUCAUUUCGAC |
| | CUCUCUGCUAGC |
| CLN9941 | GGGAGACAAGAAUAAAGCGAGUUAAUUCUACUCUUUC |
| SEQ ID NO 39 | GACCUCUCUGCUAGC |
| CLN9936 | GGGAGACAAGAAUAAAGCGAGUUACACAUACCGUCAU |
| SEQ ID NO 40 | CGCACGGUCUUAAGCAAAGAGUCGAUGAUGCUUAGCU |
| | AGAAGGGCGAAUUCGCGGCCGCUAAAUUCAUUUCGAC |
| | CUCUCUGCUAGC |
| CLN9937 | GGGAGACAAGAAUAAAGCGAGUUAUAGCACUGGCGUA |
| SEQ ID NO 41 | CUGUUCAUUAAUUAGCAAGAGUCGAUGAUGCUUAGCU |
| | AGAAGGGCGAAUUCGUUUAAACCUGCAGGACUAGUCC |
| | CUUUAGUGAGGGUUAAUUUCGACCUCUCUGCUAGC |
| CLN9938 | GGGAGACAAGAAUAAAGCGAGUUGAUAAUUACCAUUU |
| SEQ ID NO 42 | CGACCUCUCUGCUAGC |
| CLN9933 | GGGAGACAAGAAUAAAGCGAGUUCCAAAUGAGUUGUU |
| SEQ ID NO 43 | UCCUUAAAAAAACAUGAAGAGUCGAUGAUGCUUAGCU |
| | AGAAGGGCGAAUUCGCGGCCGCUAAAUUCAUUUCGAC |
| | CUCUCUGCUAGC |
| CLN9934 | GGGAGACAAGAAUAAAGCGAGUUCAAAGCAUUUCGAC |
| SEQ ID NO 44 | CUCUCUGCUAGC |
| CLN9935 | GGGAGACAAGAAUAAAGCGAGUUGUACCAAAAUGGUU |
| SEQ ID NO 45 | GUAAACCAUAUCACGCAAGAGUCGAUGAUGCUUAGCU |
| | AGAAGGGCGAAUUCGCGGCCGCUAAAUUCAUUUCGAC |
| | CUCUCUGCUAGC |
| CLN9907 | GGGAGACAAGAAUAAAGCGAGUUAUGUAAUUUCGACC |
| SEQ ID NO 46 | UCUCUGCUAGC |
| CLN9908 | GGGAGACAAGAAUAAAGCGAGUUGUAUAUCUUAACGC |
| SEQ ID NO 47 | AAGCGAGGUAAUUUGCAAGAGUCGAUGAUGCUUAGCU |
| | AGAAGGGCGAAUUCGCGGCCGCUAAAUUUCGACCUCU |
| | CUGCUAGC |
| CLN9932 | GGGAGACAAGAAUAAAGCGAGUUAUUGUUGCAUACGC |
| SEQ ID NO 48 | UUUCGACCUCUCUGCUAGC |

Clones By Round Number: 4

| | |
|---|---|
| CLN9369 | GGGAGACAAGAAUAAAGCGAGUUUACCUUCAUGAAUG |
| SEQ ID NO 49 | CAAACAGCAUGACAUAAAGAGUCGAUGAUGCUUAGCU |
| | AG |
| CLN9370 | GGGAGACAAGAAUAAAGCGAGUUUUCACUCUCUGCCA |
| SEQ ID NO 50 | UUUUCAAAAUGACACGAAGAGUCGAUGAUGCUUAGCU |
| | AG |
| CLN9366 | GGGAGACAAGAAUAAAGCGAGUUGUAUUUUUGCGCAC |
| SEQ ID NO 51 | UAGAUAACAUCUCACAAAGAGUCGAUGAUGCUUAGCU |
| | AG |
| CLN9367 | GGGAGACAAGAAUAAAGCGAGUUUGUGCAACAAAAGC |
| SEQ ID NO 52 | UUGCGACCUGUAUAUCAAGAGUCGAUGAUGCUUAGCU |
| | AG |
| CLN9368 | GGGAGACAAGAAUAAAGCGAGUUUUUCUAGCGUCACA |
| SEQ ID NO 53 | CUCAGAAUAGAAUUCAAAGAGUCGAUGAUGCUUAGCU |
| | AG |
| CLN9363 | GGGAGACAAGAAUAAAGCGAGUUAGCACAUUACUUUU |
| SEQ ID NO 54 | CUUGCAAUAGCAGUGAAAGAGUCGAUGAUGCUUAGCU |
| | AG |
| CLN9364 | GGGAGACAAGAAUAAAGCGAGUUUGUUAUAUCACGUC |
| SEQ ID NO 55 | ACUUAGUUAGCAUCACAAGAGUCGAUGAUGCUUAGCU |
| | AG |
| CLN9365 | GGGAGACAAGAAUAAAGCGAGUUGUCAUGGCAGUAUC |
| SEQ ID NO 56 | ACAACUCUAGUUAACUAAGAGUCGAUGAUGCUUAGCU |
| | AG |
| CLN9360 | GGGAGACAAGAAUAAAGCGAGUUCAGCAUACUGUUGU |
| SEQ ID NO 57 | GCACAUCAACCAUCCAAAGAGUCGAUGAUGCUUAGCU |
| | AG |
| CLN9361 | GGGAGACAAGAAUAAAGCGAGUUAGCUCAUGCCCUUU |
| SEQ ID NO 58 | AUCGUAUUGACAAUAUAAGAGUCGAUGAUGCUUAGCU |
| | AG |
| CLN9362 | GGGAGACAAGAAUAAAGCGAGUUUCCACAAAUCAAUC |
| SEQ ID NO 59 | AACCUAACGUCCUCACAAGAGUCGAUGAUGCUUAGCU |
| | AG |
| CLN9357 | GGGAGACAAGAAUAAAGCGAGUUUCAAACUACGCAGA |
| SEQ ID NO 60 | UUGCUAUCACAUCACCAAGAGUCGAUGAUGCUUAGCU |
| | AG |
| CLN9358 | GGGAGACAAGAAUAAAGCGAGUUCUCAACACUCAUGU |
| SEQ ID NO 61 | AAUAGUCAGCUUCACUAAGAGUCGAUGAUGCUUAGCU |
| | AG |
| CLN9359 | GGGAGACAAGAAUAAAGCGAGUUUCCUUGAACCAAAA |
| SEQ ID NO 62 | UAUCUAUCAUAUCACAAAGAGUCGAUGAUGCUUAGCU |
| | AG |
| CLN9354 | GGGAGACAAGAAUAAAGCGAGUUGGCAUUCGCAUUAA |
| SEQ ID NO 63 | UCAUGAUUAACUACGAAGAGUCGAUGAUGCUUAGCUA |
| | G |
| CLN9355 | GGGAGACAAGAAUAAAGCGAGUUGCCAGCUUGCCUGC |
| SEQ ID NO 64 | UCAUGAUUAACUACGAAGAGUCGGAUGAUGCUUAGCU |
| | AG |
| CLN9356 | GGGAGACAAGAAUAAAGCGAGUUCAUGGCAUUAGGUU |
| SEQ ID NO 65 | GUGAGUCCACAAAUUUAAGAGUCGAUGAUGCUUAGCU |
| | AG |
| CLN9351 | GGGAGACAAGAAUAAAGCGAGUUAUCCCAUGCACAUC |
| SEQ ID NO 66 | UAGUUUUCAUUAUCACAAGAGUCGAUGAUGCUUAGCU |
| | AG |
| CLN9352 | GGGAGACAAGAAUAAAGCGAGUUCUUGACAUUCAGUU |
| SEQ ID NO 67 | GCCUUAGACACACAUGAAGAGUCGAUGAUGCUUAGCU |
| | AG |
| CLN9353 | GGGAGACAAGAAUAAAGCGAGUUGCAAAAACACGCGC |
| SEQ ID NO 68 | AAGUUAACUUCCUCACAAGAGUCGAUGAUGCUUAGCU |
| | AG |
| CLN9348 | GGGAGACAAGAAUAAAGCGAGUUGGCAUUACUUCAAC |
| SEQ ID NO 69 | UGCAAGCGUUGUUAGUAAGAGUCGAUGAUGCUUAGCU |
| | AG |
| CLN9349 | GGGAGACAAGAAUAAAGCGAGUUCUCAUUCUUAGUUA |
| SEQ ID NO 70 | CUGUUUCGCGUGACAUAAGAGUCGAUGAUGCUUAGCU |
| | AG |
| CLN9350 | GGGAGACAAGAAUAAAGCGAGUUGGACUUACUACAGC |
| SEQ ID NO 71 | UAUCAUAAUCACGGUCAAGAGUCGAUGAUGCUUAGCU |
| | AG |
| CLN9390 | GGGAGACAAGAAUAAAGCGAGUUACAACUCAUAGUUA |
| SEQ ID NO 72 | UCAUCAUGCUCACUUCAAGAGUCGAUGAUGCUUAGCU |
| | AG |
| CLN9391 | GGGAGACAAGAAUAAAGCGAGUUGUACUUCCAAUAAU |
| SEQ ID NO 73 | CCAGCUCUCAUAUCACAAGAGUCGAUGAUGCUUAGCU |
| | AG |
| CLN9347 | GGGAGACAAGAAUAAAGCGAGUUUGACAGCUAGACCU |
| SEQ ID NO 74 | CGCUCUUGCGCUAAGUCAAGAGUCGAUGAUGCUUAGC |
| | UAG |
| CLN9387 | GGGAGACAAGAAUAAAGCGAGUUCACGCACUUUGACA |
| SEQ ID NO 75 | UUUUCACUAUGGCACAAGAGUCGAUGAUGCUUAGCUA |
| | G |
| CLN9388 | GGGAGACAAGAAUAAAGCGAGUUCAAGAUUCUCAUAU |
| SEQ ID NO 76 | CUCACUUAGGCUUUUGAAGAGUCGAUGAUGCUUAGCU |
| | AG |
| CLN9389 | GGGAGACAAGAAUAAAGCGAGUUUUCCUCACUUAAAA |
| SEQ ID NO 77 | AUGCUACCACUGUCACAAGAGUCGAUGAUGCUUAGCU |
| | AG |
| CLN9384 | GGGAGACAAGAAUAAAGCGAGUUUAUUAACAUCACCA |
| SEQ ID NO 78 | CACUUAGGACAAACGUAAGAGUCGAUGAUGCUUAGCU |
| | AG |
| CLN9385 | GGGAGACAAGAAUAAAGCGAGUUUAGCAAAAUCUGUU |
| SEQ ID NO 79 | AUGCUAAUCGAUCUGUAAGAGUCGAUGAUGCUUAGCU |
| | AG |
| CLN9386 | GGGAGACAAGAAUAAAGCGAGUUCUGCCUUACUUACA |
| SEQ ID NO 80 | AUAGCAAGAUCAGCCCAAGAGUCGAUGAUGCUUAGCU |
| | AG |
| CLN9381 | GGGAGACAAGAAUAAAGCGAGUUUGAUCUUAGUAGCU |
| SEQ ID NO 81 | UUAAUUACGCGGACAUAAGAGUCGAUGAUGCUUAGCU |
| | AG |
| CLN9382 | GGGAGACAAGAAUAAAGCGAGUUCAUGGCAUUACUUG |
| SEQ ID NO 82 | UACACUUCGCAAAGUAAGAGUCGAUGAUGCUUAGCUA |
| | G |
| CLN9383 | GGGAGACAAGAAUAAAGCGAGUUUCCACAAUUCAAUC |
| SEQ ID NO 83 | UCUAAUAACUUAUCACAAGAGUCGAUGAUGCUUAGCU |
| | AG |
| CLN9378 | GGGAGACAAGAAUAAAGCGAGUUUCCACAAACAAAAU |
| SEQ ID NO 84 | UUACUAACACUAUCACAAGAGUCGAUGAUGCUUAGCU |
| | AG |
| CLN9379 | GGGAGACAAGAAUAAAGCGAGUUCGGGUACUAAACAC |
| SEQ ID NO 85 | AACGACCUAUCCCAAAAAGAGUCGAUGAUGCUUAGCU |
| | AG |
| CLN9380 | GGGAGACAAGAAUAAAGCGAGUUAUAUAUGUCAAUCC |
| SEQ ID NO 86 | AAGCUAACUCAAUCACAAGAGUCGAUGAUGCUUAGCU |
| | AG |
| CLN9375 | GGGAGACAAGAAUAAAGCGAGUUGCUAGCGUCAUGUC |
| SEQ ID NO 87 | AUUUGAUAUCAAUCACAAGAGUCGAUGAUGCUUAGCU |
| | AG |
| CLN9376 | GGGAGACAAGAAUAAAGCGAGUUUACCUACUAUCUCA |
| SEQ ID NO 88 | AAGCUCUCACCAUCACAAGAGUCGAUGAUGCUUAGCU |
| | AG |
| CLN9377 | GGGAGACAAGAAUAAAGCGAGUUCAAACAGCAAGCUA |
| SEQ ID NO 89 | ACACGUCACCAUUUUCAAGAGUCGAUGAUGCUUAGCU |
| | AG |
| CLN9372 | GGGAGACAAGAAUAAAGCGAGUUCGAUGAUCAUUGCU |
| SEQ ID NO 90 | AAUUCGUGCUAGUAUCAAGAGUCGAUGAUGCUUAGCU |
| | AG |
| CLN9373 | GGGAGACAAGAAUAAAGCGAGUUUCAUCAUCCAAUAC |
| SEQ ID NO 91 | CUAUCUAACUAAUCACAAGAGUCGAUGAUGCUUAGCU |
| | AG |
| CLN9374 | GGGAGACAAGAAUAAAGCGAGUUCUCAUUCUUUCAAC |
| SEQ ID NO 92 | GCACAUGGAUGCUGUAAAGAGUCGAUGAUGCUUAGCU |
| | AG |
| CLN9345 | GGGAGACAAGAAUAAAGCGAGUUAAUUCACUACUAUG |
| SEQ ID NO 93 | CACAUCUGCCAAUUUCAAGAGUCGAUGAUGCUUAGCU |
| | AG |
| CLN9346 | GGGAGACAAGAAUAAAGCGAGUUAGUCGUUUCAAGCU |
| SEQ ID NO 94 | CACGCCUCUACACAGUAAGAGUCGAUGAUGCUUAGCU |
| | AG |
| CLN9371 | GGGAGACAAGAAUAAAGCGAGUUGUAAACGUGAAAUU |
| SEQ ID NO 95 | AGCUAGCACCACUACUAAGAGUCGAUGAUGCUUAGCU |
| | AG |

Clones By Round Number: 6

| | |
|---|---|
| CLN9414 | GGGAGACAAGAAUAAAGCGAGUUCAUUCUUGGCAUUC |
| SEQ ID NO 96 | UAACUUUCACUGUCACAAGAGUCGAUGAUGCUUAGCU |
| | AG |
| CLN9415 | GGGAGACAAGAAUAAAGCGAGUUACUAGCACAUCACC |
| SEQ ID NO 97 | UUUCCUCAUAUAGGCAAAGAGUCGAUGAUGCUUAGCU |
| | AG |
| CLN9412 | GGGAGACAAGAAUAAAGCGAGUUUCCGCAAAUCCAAA |
| SEQ ID NO 98 | CUACCUCACACAUCACAAGAGUCGAUGAUGCUUAGCU |
| | AG |
| CLN9413 | GGGAGACAAGAAUAAAGCGAGUUGGCCUUUAUCGGCG |
| SEQ ID NO 99 | AUCAAACACCAGAUAAAGAGUCGAUGAUGCUUAGCUA |
| | G |
| CLN9397 | TGGGAGACAAGAAUAAAGCGAGUUUGUAUUCUUUCGA |
| SEQ ID NO 100 | ACUUUCUAACACAUCACAAGAGUCGAUGAUGCUUAGC |
| | UAG |
| CLN9409 | GGGAGACAAGAAUAAAGCGAGUUCGCUCAUUGUCAAU |
| SEQ ID NO 101 | CUAGCUUCCACGUCACAAGAGUCGAUGAUGCUUAGCU |
| | AG |
| CLN9410 | GGGAGACAAGAAUAAAGCGAGUUUCCACGCACCAAUC |
| SEQ ID NO 102 | UAACUAACACUAUCACAAGAGUCGAUGAUGCUUAGCU |
| | AG |
| CLN9411 | GGGAGACAAGAAUAAAGCGAGUUUGCUUUGUCAAUCU |
| SEQ ID NO 103 | AACUAACACAAUCACAAGAGUCGAUGAUGCUUAGCUA |
| | G |
| CLN9406 | GGGAGACAAGAAUAAAGCGAGUUUGCUAUGUCAUUCU |
| SEQ ID NO 104 | AGUUCACAUCUUCACAAAGAGUCGAUGAUGCUUAGCU |
| | AG |
| CLN9407 | GGGAGACAAGAAUAAAGCGAGUUUCCACACACAGUCU |
| SEQ ID NO 105 | CUAACUAACUAUUCACAAGAGUCGAUGAUGCUUAGCU |
| | AG |
| CLN9408 | GGGAGACAAGAAUAAAGCGAGUUUAUUCUUUCGAACU |
| SEQ ID NO 106 | UUCUAACACAUCACAAGAGUCGAUGAUGCUUAGCUAG |
| CLN9404 | GGGAGACAAGAAUAAAGCGAGUUCCACACAUCCAAAC |
| SEQ ID NO 107 | UAGUUAACACCAACACAAGAGUCGAUGAUGCUUAGCU |
| | AG |
| CLN9405 | GGGAGACAAGAAUAAAGCGAGUUGGCUUAUGUCAAAC |
| SEQ ID NO 108 | UAGAUAACAUCCUUACAAGAGUCGAUGAUGCUUAGCU |
| | AG |
| CLN9401 | GGGAGACAAGAAUAAAGCGAGUUCGUUUUGUCAAACU |
| SEQ ID NO 109 | AGCUAACUACAACACAAAGAGUCGAUGAUGCUUAGCU |
| | AG |
| CLN9402 | GGGAGACAAGAAUAAAGCGAGUUACGCCAUGUCAUAC |
| SEQ ID NO 110 | UUUCUAACACAUCACAAAGAGUCGAUGAUGCUUAGCU |
| | AG |
| CLN9403 | GGGAGACAAGAAUAAAGCGAGUUUCCUCCAAGUCUUA |
| SEQ_ID NO 111 | CUAACUUUCACAUCACAAGAGUCGAUGAUGCUUAGCU |
| | AG |
| CLN9398 | GGGAGACAAGAAUAAAGCGAGUUUCCACAAAUAAUCA |
| SEQ ID NO 112 | CUUGUUAACACAUCACAAGAGUCGAUGAUGCUUAGCU |
| | AG |
| CLN9399 | GGGAGACAAGAAUAAAGCGAGUUUCCACACAUCCAAA |
| SEQ ID NO 113 | CUAGUCAACUUAUCACAAGAGUCGAUGAUGCUUAGCU |
| | AG |
| CLN9400 | GGGAGACAAGAAUAAAGCGAGUUUUUCUCCACACACA |
| SEQ ID NO 114 | CCAGUUAACUUAUCACAAGAGUCGAUGAUGCUUAGCU |
| | AG |
| CLN9395 | GGGAGACAAGAAUAAAGCGAGUUCCACACCAUCCAAC |
| SEQ ID NO 115 | UAACUAACUUAUCACAAGAGUCGAUGAUGCUUAGCUA |
| | G |
| CLN9396 | GGGAGACAAGAAUAAAGCGAGUUGUGCUUCUAUCAAU |
| SEQ ID NO 116 | CUAGUUAACUUAUCACAAGAGUCGAUGAUGCUUAGCU |
| | AG |
| CLN9447 | GGGAGACAAGAAUAAAGCGAGUUCUAUUCUUUGCAAC |
| SEQ ID NO 117 | UAGCUGACACUAUCACAAGAGUCGAUGAUGCUUAGCU |
| | AG |
| CLN9448 | GGGAGACAAGAAUAAAGCGAGUUAUUGCAUGUUAGUC |
| SEQ ID NO 118 | CUCGUAGACUCUCCAUAAGAGUCGAUGAUGCUUAGCU |
| | AG |
| CLN9394 | GGGAGACAAGAAUAAAGCGAGUUUCCACGAUCCACAA |
| SEQ ID NO 119 | CUAUCUAACAUAUCACAAGAGUCGAUGAUGCUUAGCU |
| | AG |
| CLN9445 | GGGAGACAAGAAUAAAGCGAGUUUCCGCGAAUAACAU |
| SEQ ID NO 120 | CUAACUAACUAAUCACAAGAGUCGAUGAUGCUUAGCU |
| | AG |
| CLN9446 | GGGAGACAAGAAUAAAGCGAGUUCACGAUCUACACCG |
| SEQ ID NO 121 | AUUAUCUUAUUGGUGUAAGAGUCGAUGAUGCUUAGCU |
| | AG |
| CLN9442 | GGGAGACAAGAAUAAAGCGAGUUUGUCAAACGGCGUC |
| SEQ ID NO 122 | ACACUCUUCUAUAAUGUAAGAGUCGAUGAUGCUUAGC |
| | UAG |
| CLN9443 | GGGAGACAAGAAUAAAGCGAGUUUGCUAUGUCUAUAC |
| SEQ ID NO 123 | UAGCUUCCAUUGUCACAAGAGUCGAUGAUGCUUAGCU |
| | AG |
| CLN9444 | GGGAGACAAGAAUAAAGCGAGUUUCCACAAACGCAAU |
| SEQ ID NO 124 | CUAGCUAUCAUAUCACAAGAGUCGAUGAUGCUUAGCU |
| | AG |
| CLN9439 | GGGAGACAAGAAUAAAGCGAGUUACGCUCAUGUCAUC |
| SEQ ID NO 125 | UAGCUUCCAACGUCACAAGAGUCGAUGAUGCUUAGCU |
| | AG |
| CLN9440 | GGGAGACAAGAAUAAAGCGAGUUGUUUGUCAAUCUAG |
| SEQ ID NO 126 | CUUCCAAUAUCACAAGAGUCGAUGAUGCUUAGCUAG |
| CLN9441 | GGGAGACAAGAAUAAAGCGAGUUUUGUCAUCUAUCGU |
| SEQ ID NO 127 | CACGCCUCUACACAGUAAGAGUCGAUGAUGCUUAGCU |
| | AG |
| CLN9437 | GGGAGACAAGAAUAAAGCGAGUUUCCGCGAAACACAU |
| SEQ ID NO 128 | CUAGCUCACAUUUCACAAGAGUCGAUGAUGCUUAGCU |
| | AG |
| CLN9438 | GGGAGACAAGAAUAAAGCGAGUUUACGCAUUGUCAUC |
| SEQ ID NO 129 | CUAGUUAACAUAUCACAAGAGUCGAUGAUGCUUAGCU |
| | AG |
| CLN9436 | GGGAGACAAGAAUAAAGCGAGUUGUAUUCUUUUCACA |
| SEQ ID NO 130 | CUAUCUUCCAUGACACAAGAGUCGAUGAUGCUUAGCU |
| | AG |
| CLN9433 | GGGAGACAAGAAUAAAGCGAGUUUCUGCAACCACAAA |
| SEQ ID NO 131 | CUAAUUAACACAUCACAAGAGUCGAUGAUGCUUAGCU |
| | AG |
| CLN9434 | GGGAGACAAGAAUAAAGCGAGUUGUGUUCUUGCAAAC |
| SEQ ID NO 132 | UAACUAACGUGAUCACAAGAGUCGAUGAUGCUUAGCU |
| | AG |
| CLN9435 | GGGAGACAAGAAUAAAGCGAGUUGUAUUCUUGCAAAC |
| SEQ ID NO 133 | UAUCUAUCAUUGUUACAAGAGUCGAUGAUGCUUAGCU |
| | AG |
| CLN9430 | GGGAGACAAGAAUAAAGCGAGUUAUAUUCUUAUCAAC |
| SEQ ID NO 134 | UAGUUGACGCCAACACAAGAGUCGAUGAUGCUUAGCU |
| | AG |
| CLN9431 | GGGAGACAAGAAUAAAGCGAGUUACGCGUUGUCAAUC |
| SEQ ID NO 135 | UAGUUAACAUAAUCACAAGAGUCGAUGAUGCUUAGCU |
| | AG |
| CLN9432 | GGGAGACAAGAAUAAAGCGAGUUAUCCGCACCAACCA |
| SEQ ID NO 136 | UCUACUAUCAUAUCACAAGAGUCGAUGAUGCUUAGCU |
| | AG |
| CLN9427 | GGGAGACAAGAAUAAAGCGAGUUUGCCAUUGUCACCU |
| SEQ ID NO 137 | AGUUAACAUCAACACAAAGAGUCGAUGAUGCUUAGCU |
| | AG |
| CLN9428 | GGGAGACAAGAAUAAAGCGAGUUUGCUUUUGUCACAC |
| SEQ ID NO 138 | UAUUUAUCCCAUCACAAAGAGUCGAUGAUGCUUAGCU |
| | AG |
| CLN9429 | GGGAGACAAGAAUAAAGCGAGUUUCCACGAACCAAUC |
| SEQ ID NO 139 | UAGCUUCCAACUUCACAAGAGUCGAUGAUGCUUAGCU |
| | AG |
| CLN9425 | GGGAGACAAGAAUAAAGCGAGUUUCCGCAAAAUCAAU |
| SEQ ID NO 140 | CUAACUAACAUAUCACAAGAGUCGAUGAUGCUUAGCU |
| | AG |
| CLN9426 | GGGAGACAAGAAUAAAGCGAGUUAUAUUAAUGUCGGC |
| SEQ ID NO 141 | CUAGGUAACACAUCACAAGAGUCGAUGAUGCUUAGCU |
| | AG |
| CLN9422 | GGGAGACAAGAAUAAAGCGAGUUUCAGCACAAACAAA |
| SEQ ID NO 142 | CUAGCUCACAUUUCACAAGAGUCGAUGAUGCUUAGCU |
| | AG |
| CLN9423 | GGGAGACAAGAAUAAAGCGAGUUGUGCUACCAUCAAG |
| SEQ ID NO 143 | CUAGCUAACUUAUCACAAGAGUCGAUGAUGCUUAGCU |
| | AG |
| CLN9424 | GGGAGACAAGAAUAAAGCGAGUUGUAUUCUUAUCAUG |
| SEQ ID NO 144 | CUUUCUAACACAUCACAAGAGUCGAUGAUGCUUAGCU |
| | AG |
| CLN9420 | GGGAGACAAGAAUAAAGCGAGUUUCUUCACAACAUAA |
| SEQ ID NO 145 | CUAGUUUCCAUGUCACAAGAGUCGAUGAUGCUUAGCU |
| | AG |
| CLN9421 | GGGAGACAAGAAUAAAGCGAGUUUCCUCAAAUACAGC |
| SEQ ID NO 146 | UAGCUUCCACCACCACAAGAGUCGAUGAUGCUUAGCU |
| | AG |
| CLN9417 | GGGAGACAAGAAUAAAGCGAGUUAGCUAUGUCAAACU |
| SEQ ID NO 147 | AGCUCUCCUUAUCACAAGAGUCGAUGAUGCUUAGCUA |
| | G |
| CLN9418 | GGGAGACAAGAAUAAAGCGAGUUGUUCUCACAAACCA |
| SEQ ID NO 148 | CUAGCUCUCAUAUCACAAGAGUCGAUGAUGCUUAGCU |
| | AG |
| CLN9419 | GGGAGACAAGAAUAAAGCGAGUUAUAUUGCUGUCAAA |
| SEQ ID NO 149 | CUAUUUAACUUAUCACAAGAGUCGAUGAUGCUUAGCU |
| | AG |
| CLN9392 | GGGAGACAAGAAUAAAGCGAGUUAUAUUCUUUGCAAC |
| SEQ ID NO 150 | UUGCUAACACUAUCACAAGAGUCGAUGAUGCUUAGCU |
| | AG |
| CLN9393 | GGGAGACAAGAAUAAAGCGAGUUGUAAUCUUGUCAAC |
| SEQ ID NO 151 | UUAGUUAACUUAUCACAAGAGUCGAUGAUGCUUAGCU |
| | AG |
| CLN9416 | GGGAGACAAGAAUAAAGCGAGUUGAUCAUCCAAACAC |
| SEQ ID NO 152 | UAUCUUCCAUCCUCACAAGAGUCGAUGAUGCUUAGCU |
| | AG |

Clones By Round Number: 8

| | |
|---|---|
| CLN10231 | GGGAGACAAGAAUAAAGCGAGUUCACUACCUGUAUGU |
| SEQ ID NO 153 | GUCAAUAGAUCCAAUCAAGAGUCGAUGAUGCUUAGCU |
| | AG |
| CLN10228 | GGGAGACAAGAAUAAAGCGAGUUGGACCUUCUCGUUU |
| SEQ ID NO 154 | AAAUCAGGUUAGCGUCAAGAGUCGAUGAUGCUUAGCU |
| | AG |
| CLN10229 | GGGAGACAAGAAUAAAGCGAGUUCCCACAAAUGCCUU |
| SEQID NO 155 | CUAGCUAACUUAUCACAAGAGUCGAUGAUGCUUAGCU |
| | AG |
| CLN10230 | GGGAGACAAGAAUAAAGCGAGUUGGCCUCCUAAUAUA |
| SEQ ID NO 156 | CGCCUAACAACGUCACAAGAGUCGAUGAUGCUUAGCU |
| | AG |
| CLN10225 | GGGAGACAAGAAUAAAGCGAGUUGUAUUCUUAUCGCA |
| SEQ ID NO 157 | CUAGCUCACUGAUCACAAGAGUCGAUGAUGCUUAGCU |
| | AG |
| CLN10226 | GGGAGACAAGAAUAAAGCGAGUUGUUCUUCCUAACAC |
| SEQ ID NO 158 | UAGUUUCCAUUUUCACAAGAGUCGAUGAUGCUUAGCU |
| | AG |
| CLN10227 | GGGAGACAAGAAUAAAGCGAGUUAGCUUUGUCACAAC |
| SEQ ID NO 159 | UAUUUAACUUUGUCACAAGAGUCGAUGAUGCUUAGCU |
| | AG |
| CLN10222 | GGGAGACAAGAAUAAAGCGAGUUGUCAUAAAGCUUUU |
| SEQ ID NO 160 | GUGAUCGCUCAUAGUCAAGAGUCGAUGAUGCUUAGCU |
| | AG |
| CLN10223 | GGGAGACAAGAAUAAAGCGAGUUCUCUUCACAUACAA |
| SEQID NO 161 | GCAGUUAACACAUCACAAGAGUCGAUGAUGCUUAGCU |
| | AG |
| CLN10224 | GGGAGACAAGAAUAAAGCGAGUUUCCACAACAACCUU |
| SEQ ID NO 162 | CUAACUAACUUAUCACAAGAGUCGAUGAUGCUUAGCU |
| | AG |
| CLN10220 | GGGAGACAAGAAUAAAGCGAGUUUCCACGUCAACCAU |
| SEQ ID NO 163 | CUAUAUAACAUAUCACAAGAGUCGAUGAUGCUUAGCU |
| | AG |
| CLN9397 | GGGAGACAAGAAUAAAGCGAGUUUGUAUUCUUUCGAA |
| SEQ ID NO 164 | CUUUCUAACACAUCACAAGAGUCGAUGAUGCUUAGCU |
| | AG |
| CLN10221 | GGGAGACAAGAAUAAAGCGAGUUGAUCUAACAAGUAU |
| SEQ ID NO 165 | UAGUAUAUGCUAUGGCAAGAGUCGAUGAUGCUUAGCU |
| | AG |
| CLN10217 | GGGAGACAAGAAUAAAGCGAGUUAUAUUCUUAUCAAC |
| SEQ ID NO 166 | AUAGUUAACUUAUCACAAGAGUCGAUGAUGCUUAGCU |
| | AG |
| CLN10218 | GGGAGACAAGAAUAAAGCGAGUUGGCCUUACAUUGUA |
| SEQ ID NO 167 | CUAUAAUUCAUAUGUCAAGAGUCGAUGAUGCUUAGCU |
| | AG |
| CLN10219 | GGGAGACAAGAAUAAAGCGAGUUUGCUAUGUCAAUCU |
| SEQ ID NO 168 | AGCUUCCAUCAACACAAAGAGUCGAUGAUGCUUAGCU |
| | AG |
| CLN9406 | GGGAGACAAGAAUAAAGCGAGUUUGCUAUGUCAUUCU |
| SEQ ID NO 169 | AGUUCACAUCUUCACAAAGAGUCGAUGAUGCUUAGCU |
| | AG |
| CLN10215 | GGGAGACAAGAAUAAAGCGAGUUUGUAUUCUUUCGGU |
| SEQ ID NO 170 | CUUGCUAACUUAUCACAAGAGUCGAUGAUGCUUAGCU |
| | AG |
| CLN10216 | GGGAGACAAGAAUAAAGCGAGUUGACGCUAUGUCAUC |
| SEQ ID NO 171 | UAGCUAUCAACGUCACAAGAGUCGAUGAUGCUUAGCU |
| | AG |
| CLN10212 | GGGAGACAAGAAUAAAGCGAGUUUCCACACACAGCAA |
| SEQ ID NO 172 | CUAGCUCACACAUCACAAGAGUCGAUGAUGCUUAGCU |
| | AG |
| CLN10213 | GGGAGACAAGAAUAAAGCGAGUUUCCACAAAUCAAAC |
| SEQ ID NO 173 | CUAUCUAACCCAUCACAAGAGUCGAUGAUGCUUAGCU |
| | AG |
| CLN10214 | GGGAGACAAGAAUAAAGCGAGUUUCAUCAAAAUCACA |
| SEQ ID NO 174 | CUAGCCGACACAUCACAAGAGUCGAUGAUGCUUAGCU |
| | AG |
| CLN10210 | GGGAGACAAGAAUAAAGCGAGUUUGCUUUAUCAAUCU |
| SEQ ID NO 175 | AGCUCACAUCUCACCUAAGAGUCGAUGAUGCUUAGCU |
| | AG |
| CLN10211 | GGGAGACAAGAAUAAAGCGAGUUUCCACAAAUACAAC |
| SEQ ID NO 176 | CUAUCUACCUUAUCACAAGAGUCGAUGAUGCUUAGCU |
| | AG |
| CLN10272 | GGGAGACAAGAAUAAAGCGAGUUUCAGCAAACCAACC |
| SEQ ID NO 177 | AUAGCUUUCAAGUCACAAGAGUCGAUGAUGCUUAGCU |
| | AG |
| CLN10273 | GGGAGACAAGAAUAAAGCGAGUUCAGCUGUCUUAAUA |
| SEQ ID NO 178 | AUCUAUCUCGUACACGAAGAGUCGAUGAUGCUUAGCU |
| | AG |
| CLN10274 | GGGAGACAAGAAUAAAGCGAGUUUCCACAAAACAUCC |
| SEQ ID NO 179 | CUAACUAACUAGUCACAAGAGUCGAUGAUGCUUAGCU |
| | AG |
| CLN10269 | GGGAGACAAGAAUAAAGCGAGUUAACUGUAGGCUCAC |
| SEQ ID NO 180 | GAUCUUAUUCAUCAGUAAGAGUCGAUGAUGCUUAGCU |
| | AG |
| CLN10270 | GGGAGACAAGAAUAAAGCGAGUUUUCCACAAAUCUUC |
| SEQ ID NO 181 | CUAGUUAACUUAUCACAAGAGUCGAUGAUGCUUAGCU |
| | AG |
| CLN10271 | GGGAGACAAGAAUAAAGCGAGUUAGCUGUGUCAUGCU |
| SEQ ID NO 182 | AGUUAACCUCUCACUUAAGAGUCGAUGAUGCUUAGCU |
| | AG |
| CLN10268 | GGGAGACAAGAAUAAAGCGAGUUAAACUAGUGCUAAC |
| SEQ ID NO 183 | UCAACACACUUAAUAAAGAGUCGAUGAUGCUUAGCUA |
| | G |
| CLN10242 | GGGAGACAAGAAUAAAGCGAGUUACGCUAUGUCAAAC |
| SEQ ID NO 184 | UAGCUAACUACAACACAAGAGUCGAUGAUGCUUAGCU |
| | AG |
| CLN10266 | GGGAGACAAGAAUAAAGCGAGUUUCCACAAAGCUAAA |
| SEQ ID NO 185 | CCAGCUAACUUAUCACAAGAGUCGAUGAUGCUUAGCU |
| | AG |
| CLN10267 | GGGAGACAAGAAUAAAGCGAGUUUGCUAUUGUCAUAC |
| SEQ ID NO 186 | UAGUUAACAGAAUCACAAGAGUCGAUGAUGCUUAGCU |
| | AG |
| CLN10263 | GGGAGACAAGAAUAAAGCGAGUUUCCACAAAGUCAAA |
| SEQ ID NO 187 | CUAACCAGCACUUCACAAGAGUCGAUGAUGCUUAGCU |
| | AG |
| CLN10264 | GGGAGACAAGAAUAAAGCGAGUUGCGCAUUACGAAUC |
| SEQ ID NO 188 | UACACCAAAGGGUCGGAAGAGUCGAUGAUGCUUAGCU |
| | AG |
| CLN10265 | GGGAGACAAGAAUAAAGCGAGUUUCUUCAAAUCACUC |
| SEQ ID NO 189 | CUAGUUAACAUGUCACAAGAGUCGAUGAUGCUUAGCU |
| | AG |
| CLN9411 | GGGAGACAAGAAUAAAGCGAGUUUGCUUUGUCAAUCU |
| SEQ ID NO 190 | AACUAACACAAUCACAAGAGUCGAUGAUGCUUAGCUA |
| | G |
| CLN10244 | GGGAGACAAGAAUAAAGCGAGUUUGUAUUCUUUUCAA |
| SEQ ID NO 191 | UCUAGCAACUUAUCACAAGAGUCGAUGAUGCUUAGCU |
| | AG |
| CLN10260 | GGGAGACAAGAAUAAAGCGAGUUUGCGUUGUCAAUCU |
| SEQ ID NO 192 | AGCUUUCAUCAACACUAAGAGUCGAUGAUGCUUAGCU |
| | AG |
| CLN10261 | GGGAGACAAGAAUAAAGCGAGUUACGCUUUGUCAAAC |
| SEQ ID NO 193 | UAAUUAACUACAUCACAAGAGUCGAUGAUGCUUAGCU |
| | AG |
| CLN10262 | GGGAGACAAGAAUAAAGCGAGUUUCAGCAACCAAUAC |
| SEQ ID NO 194 | CUAGCUUCCAUCACACAAGAGUCGAUGAUGCUUAGCU |
| | AG |
| CLN10257 | GGGAGACAAGAAUAAAGCGAGUUAUAUUCUGUCAAUC |
| SEQ ID NO 195 | UAGUUUCCAUCUCACUAAGAGUCGAUGAUGCUUAGCU |
| | AG |
| CLN10258 | GGGAGACAAGAAUAAAGCGAGUUUCCUCCAACCAACU |
| SEQ ID NO 196 | CUAGCUCACUUAUCACAAGAGUCGAUGAUGCUUAGCU |
| | AG |
| CLN10259 | GGGAGACAAGAAUAAAGCGAGUUAUCGUUUCGUCAAA |
| SEQ ID NO 197 | CUAGGUAACAUAUCACAAGAGUCGAUGAUGCUUAGCU |
| | AG |
| CLN10254 | GGGAGACAAGAAUAAAGCGAGUUUGCUCUCUCAACAC |
| SEQ ID NO 198 | CUAUCUAACUAGUCACAAGAGUCGAUGAUGCUUAGCU |
| | AG |
| CLN10255 | GGGAGACAAGAAUAAAGCGAGUUAGCUAUGUCAAACU |
| SEQ ID NO 199 | AGCUCUCCUUAUCACAAAGAGUCGAUGAUGCUUAGCU |
| | AG |
| CLN10256 | GGGAGACAAGAAUAAAGCGAGUUUCCACGAUCCAUCA |
| SEQ ID NO 200 | ACUAGCAACUUAUCACAAGAGUCGAUGAUGCUUAGCU |
| | AG |
| CLN10251 | GGGAGACAAGAAUAAAGCGAGUUUCCACGACAGCAAU |
| SEQ ID NO 201 | CUAGCUCUCAUAUCACAAGAGUCGAUGAUGCUUAGCU |
| | AG |
| CLN10252 | GGGAGACAAGAAUAAAGCGAGUUUAAUUGUUGUCAAA |
| SEQ ID NO 202 | CUAACUUCCAUAUCACAAGAGUCGAUGAUGCUUAGCU |
| | AG |
| CLN10253 | GGGAGACAAGAAUAAAGCGAGUUAUAUUCUGACAAAC |
| SEQ ID NO 203 | UAGCUAACGACAUUACAAGAGUCGAUGAUGCUUAGCU |
| | AG |
| CLN10248 | GGGAGACAAGAAUAAAGCGAGUUGUGCUUCCAAAAUU |
| SEQ ID NO 204 | CUAACUAACUUAUCACAAGAGUCGAUGAUGCUUAGCU |
| | AG |
| CLN10249 | GGGAGACAAGAAUAAAGCGAGUUAGCCAUUGUCAAAC |
| SEQ ID NO 205 | UAGUUCACACAAUCACAAGAGUCGAUGAUGCUUAGCU |
| | AG |
| CLN10250 | GGGAGACAAGAAUAAAGCGAGUUUGAUCUCUCGCCAA |
| SEQ ID NO 206 | CUAGUUAACUUAUCACAAGAGUCGAUGAUGCUUAGCU |
| | AG |
| CLN10246 | GGGAGACAAGAAUAAAGCGAGUUGCAAGAAAACACGA |
| SEQ ID NO 207 | GCACAUUUACCUACAAAAGAGUCGAUGAUGCUUAGCU |
| | AG |
| CLN10247 | GGGAGACAAGAAUAAAGCGAGUUGCAACUAAAUAUAG |
| SEQ ID NO 208 | CACGCCGACAUUGUAAAAGAGUCGAUGAUGCUUAGCU |
| | AG |
| CLN10243 | GGGAGACAAGAAUAAAGCGAGUUUGCAUGUGUCACAC |
| SEQ ID NO 209 | UAGUUAACACGAUCACAAGAGUCGAUGAUGCUUAGCU |
| | AG |
| CLN10245 | GGGAGACAAGAAUAAAGCGAGUUUGCUGUGUCAUUCU |
| SEQ ID NO 210 | AAGCUAACAUAGUCACAAGAGUCGAUGAUGCUUAGCU |
| | AG |
| CLN10240 | GGGAGACAAGAAUAAAGCGAGUUUACGCUCUGUCAUA |
| SEQ ID NO 211 | CUUUCUAACUUAUCACAAGAGUCGAUGAUGCUUAGCU |
| | AG |
| CLN10241 | GGGAGACAAGAAUAAAGCGAGUUCUAUUCUGUCAUAC |
| SEQ ID NO 212 | UAGCUAACACUUCACAAAGAGUCGAUGAUGCUUAGCU |
| | AG |
| CLN10237 | GGGAGACAAGAAUAAAGCGAGUUCACUCAGACGUUCA |
| SEQ ID NO 213 | CAUAACGCUGUUCGAUAAGAGUCGAUGAUGCUUAGCU |
| | AG |
| CLN10238 | GGGAGACAAGAAUAAAGCGAGUUCUUCCUAAACUGCC |
| SEQ ID NO 214 | GAAACUGCACAAUAUCAAGAGUCGAUGAUGCUUAGCU |
| | AG |
| CLN10239 | GGGAGACAAGAAUAAAGCGAGUUGGCAUUAUUACGUA |
| SEQ ID NO 215 | CUCACCAUACGCUAAUAAGAGUCGAUGAUGCUUAGCU |
| | AG |
| CLN10235 | GGGAGACAAGAAUAAAGCGAGUUGAUCUGAUUUCAAG |
| SEQ ID NO 216 | UUGUCAUUGCAUAAUCAAGAGUCGAUGAUGCUUAGCU |
| | AG |
| CLN10236 | GGGAGACAAGAAUAAAGCGAGUUUGCUAUGUCAAUCU |
| SEQ ID NO 217 | AGCUUCCAUCAGCACUAAGAGUCGAUGAUGCUUAGCU |
| | AG |
| CLN10233 | GGGAGACAAGAAUAAAGCGAGUUACACUAACAGGCUC |
| SEQ ID NO 218 | UCUUCUCUACUACAGUAAGAGUCGAUGAUGCUUAGCU |
| | AG |
| CLN10234 | GGGAGACAAGAAUAAAGCGAGUUCACGCUCUGUCAAU |
| SEQ ID NO 219 | CUAUCUAACUUAUCACAAGAGUCGAUGAUGCUUAGCU |
| | AG |
| CLN10208 | GGGAGACAAGAAUAAAGCGAGUUGUAUUCUUUCGCAC |
| SEQ ID NO 220 | UAGCCAACAUAAUCACAAGAGUCGAUGAUGCUUAGCU |
| | AG |
| CLN10209 | GGGAGACAAGAAUAAAGCGAGUUACGCUCAUGUCAUA |
| SEQ ID NO 221 | CUAAUUAACAUAUCACAAGAGUCGAUGAUGCUUAGCU |
| | AG |
| CLN10232 | GGGAGACAAGAAUAAAGCGAGUUGGACUUCCAAACAU |
| SEQ ID NO 222 | CUCGUUAACACAUCACAAGAGUCGAUGAUGCUUAGCU |
| | AG |

Clones By Round Number: 9

| | |
|---|---|
| CLN9397 | GGGAGACAAGAAUAAAGCGAGUUUGUAUUCUUUCGAA |
| SEQ ID NO 223 | CUUUCUAACACAUCACAAGAGUCGAUGAUGCUUAGCU |
| | AG |
| CLN10217 | GGGAGACAAGAAUAAAGCGAGUUAUAUUCUUAUCAAC |
| SEQ ID NO 224 | AUAGUUAACUUAUCACAAGAGUCGAUGAUGCUUAGCU |
| | AG |
| CLN11751 | GGGAGACAAGAAUAAAGCGAGUUGAACUAGCAUCAAU |
| SEQ ID NO 225 | CUCUACAAUACAUUGGAAGAGUCGAUGAUGCUUAGCU |
| | AG |
| CLN10235 | GGGAGACAAGAAUAAAGCGAGUUGAUCUGAUUUCAAG |
| SEQ ID NO 226 | UUGUCAUUGCAUAAUCAAGAGUCGAUGAUGCUUAGCU |
| | AG |
| CLN11752 | GGGAGACAAGAAUAAAGCGAGUUGGCCUUACAUUGUA |
| SEQ ID NO 227 | CAUAAUUCAUAUGUCAAGAGUCGAUGAUGCUUAGCUA |
| | G |
| CLN9406 | GGGAGACAAGAAUAAAGCGAGUUUGCUAUGUCAUUCU |
| SEQ ID NO 228 | AGUUCACAUCUUCACAAAGAGUCGAUGAUGCUUAGCU |
| | AG |
| CLN11750 | GGGAGACAAGAAUAAAGCGAGUUCCGCAGUGCAAGGC |
| SEQ ID NO 229 | UCAAGGUCUACCACUGAAGAGUCGAUGAUGCUUAGCU |
| | AG |
| CLN11749 | GGGAGACAAGAAUAAAGCGAGUUGAUGCAUUUAGCCA |
| SEQ ID NO 230 | GGAUAGUCCCCUAUAGUAAUAAUUGAUGCAUUUAGCC |
| | AGGAUAGUCCCUAUAGUAAUAAUUGAUGCAUUUAGCC |
| | AGGAUAGUCCCCUAUAGUAAUAAUUGAUGCAUUUAGC |
| | CAGGAUAGUUCCCAUAGUAAGAGUCGAUGAUGCUUAG |
| | CUAG |
| CLN11748 | GGGAGACAAGAAUAAAGCGAGUUCCGUAGUGCAAGGC |
| SEQ ID NO 231 | UCAAGGUCUACCACUGAAGAGUCGAUGAUGCUUAGCU |
| | AG |
| CLN11746 | GGGAGACAAGAAUAAAGCGAGUUUUCACCACAAACAA |
| SEQ ID NO 232 | CUAACUAACUUAUCACAAGAGUCGAUGAUGCUUAGCU |
| | AG |
| CLN11747 | GGGAGACAAGAAUAAAGCGAGUUGGCCUUCCAUCAAA |
| SEQ ID NO 233 | CAGCUUUCAAUAUCACAAGAGUCGAUGAUGCUUAGCU |
| | AG |
| CLN10213 | GGGAGACAAGAAUAAAGCGAGUUUCCACAAAUCAAAC |
| SEQ ID NO 234 | CUAUCUAACCCAUCACAAGAGUCGAUGAUGCUUAGCU |
| | AG |
| CLN11744 | GGGAGACAAGAAUAAAGCGAGUUUUUACGUUCUUCAA |
| SEQ ID NO 235 | GACUUGACUGUCUAUGAAAGAGUCGAUGAUGCUUAGC |
| | UAG |
| CLN11745 | GGGAGACAAGAAUAAAGCGAGUUAUCCACAAAUCAAC |
| SEQ ID NO 236 | CAAGUUAACACAUCACAAGAGUCGAUGAUGCUUAGCU |
| | AG |
| CLN11999 | GGGAGACAAGAAUAAAGCGAGUUGGCCUUCCAUCAAA |
| SEQ ID NO 237 | CUGCUUUCAAUAUCACAAGAGUCGAUGAUGCUUAGCU |
| | AG |
| CLN11995 | GGGAGACAAGAAUAAAGCGAGUUUCCACAAAGCCAAA |
| SEQ ID NO 238 | CCAACUAACUUAUCACAAGAGUCGAUGAUGCUUAGCU |
| | AG |
| CLN11743 | GGGAGACAAGAAUAAAGCGAGUUUCCGCACAAAGCAA |
| SEQ ID NO 239 | CUAGCUAACAUAUCACAAGAGUCGAUGAUGCUUAGCU |
| | AG |
| CLN11997 | GGGAGACAAGAAUAAAGCGAGUUUCCACACAUCCAAA |
| SEQ ID NO 240 | CUAGUCGACUUAUCACAAAGAGUCGAUGAUGCUUAGC |
| | UAG |
| CLN11998 | GGGAGACAAGAAUAAAGCGAGUUUCCACAAAUAAAAU |
| SEQ ID NO 241 | CUAGUUCACUUAUCACAAGAGUCGAUGAUGCUUAGCU |
| | AG |
| CLN10214 | GGGAGACAAGAAUAAAGCGAGUUUCAUCAAAAUCACA |
| SEQ ID NO 242 | CUAGCCGACACAUCACAAGAGUCGAUGAUGCUUAGCU |
| | AG |
| CLN11996 | GGGAGACAAGAAUAAAGCGAGUUCCUUCAAAUCAAAC |
| SEQ ID NO 243 | CUAACUAACUUAUCACAAGAGUCGAUGAUGCUUAGCU |
| | AG |
| CLN9399 | GGGAGACAAGAAUAAAGCGAGUUUCCACACAUCCAAA |
| SEQ ID NO 244 | CUAGUCAACUUAUCACAAGAGUCGAUGAUGCUUAGCU |
| | AG |
| CLN11992 | GGGAGACAAGAAUAAAGCGAGUUUUCCUCACAUCAAU |
| SEQ ID NO 245 | CAGCUAACUUAUCACAAGAGUCGAUGAUGCUUAGCUA |
| | G |
| CLN11993 | GGGAGACAAGAAUAAAGCGAGUUUCCACAAAUCCAAC |
| SEQ ID NO 246 | CUAGCAAACCCAUCACAAGAGUCGAUGAUGCUUAGCU |
| | AG |
| CLN11994 | GGGAGACAAGAAUAAAGCGAGUUUCCACACAUCCAAA |
| SEQ ID NO 247 | CUAGACAACUUAUCACAAGAGUCGAUGAUGCUUAGCU |
| | AG |
| CLN9438 | GGGAGACAAGAAUAAAGCGAGUUUACGCAUUGUCAUC |
| SEQ ID NO 248 | CUAGUUAACAUAUCACAAGAGUCGAUGAUGCUUAGCU |
| | AG |
| CLN11990 | GGGAGACAAGAAUAAAGCGAGUUUCCACGCAAACAAA |
| SEQ ID NO 249 | CUAUCUUACACAUCACAAGAGUCGAUGAUGCUUAGCU |
| | AG |
| CLN11991 | GGGAGACAAGAAUAAAGCGAGUUUGUCAUAUCUAAGG |
| SEQ ID NO 250 | CACAUAUACCUAUCCAAAGAGUCGAUGAUGCUUAGCU |
| | AG |
| CLN11988 | GGGAGACAAGAAUAAAGCGAGUUUCCACGAAAUACAU |
| SEQ ID NO 251 | CCCAGCUAGCAAUCACAAGAGUCGAUGAUGCUUAGCU |
| | AG |
| CLN11989 | GGGAGACAAGAAUAAAGCGAGUUUGCAUUGUCAUAUU |
| SEQ ID NO 252 | AGCUAACAUCAACACAAAGAGUCGAUGAUGCUUAGCU |
| | AG |
| CLN9394 | GGGAGACAAGAAUAAAGCGAGUUUCCACGAUCCACAA |
| SEQ ID NO 253 | CUAUCUAACAUAUCACAAGAGUCGAUGAUGCUUAGCU |
| | AG |
| CLN11986 | GGGAGACAAGAAUAAAGCGAGUUUCCACAAAUACCAU |
| SEQ ID NO 254 | CUAACUAACAAAUCACAAGAGUCGAUGAUGCUUAGCU |
| | AG |
| CLN11987 | GGGAGACAAGAAUAAAGCGAGUUGCUUUUGUCAAACU |
| SEQ ID NO 255 | AGCUAUCACUAUCACAAAGAGUCGAUGAUGCUUAGCU |
| | AG |
| CLN11984 | GGGAGACAAGAAUAAAGCGAGUUUCCACAAAUCAACA |
| SEQ ID NO 256 | CAGUCUAACUUAUCACAAGAGUCGAUGAUGCUUAGCU |
| | AG |
| CLN10224 | GGGAGACAAGAAUAAAGCGAGUUUCCACAACAACCUU |
| SEQ ID NO 257 | CUAACUAACUUAUCACAAGAGUCGAUGAUGCUUAGCU |
| | AG |
| CLN11985 | GGGAGACAAGAAUAAAGCGAGUUUCCACUAAUCAUCA |
| SEQ ID NO 258 | CUAGCUCACACAUCACAAGAGUCGAUGAUGCUUAGCU |
| | AG |
| CLN11982 | GGGAGACAAGAAUAAAGCGAGUUCUCAGCACAACCAA |
| SEQ ID NO 259 | CUAGCUCACUAAUCACAAGAGUCGAUGAUGCUUAGCU |
| | AG |
| CLN11983 | GGGAGACAAGAAUAAAGCGAGUUUCAGCAAACAACCA |
| SEQ ID NO 260 | CUUUCUAACAUAUCACAAGAGUCGAUGAUGCUUAGCU |
| | AG |
| CLN11941 | GGGAGACAAGAAUAAAGCGAGUUUCCACAAUUCAACA |
| SEQ ID NO 261 | CUAACUAACUUAUCACAAGAGUCGAUGAUGCUUAGCU |
| | AG |
| CLN11979 | GGGAGACAAGAAUAAAGCGAGUUUCACCAACAACAUC |
| SEQID NO 262 | UAUCUAACUUAUCACAAAGAGUCGAUGAUGCUUAGCU |
| | AG |
| CLN11980 | GGGAGACAAGAAUAAAGCGAGUUUCCUUACACACACA |
| SEQ ID NO 263 | CACGUUAACUAAUCACAAGAGUCGAUGAUGCUUAGCU |
| | AG |
| CLN11981 | GGGAGACAAGAAUAAAGCGAGUUUCCACAAGCCAAAC |
| SEQ ID NO 264 | CUAACUAACAUAUCGCAAGAGUCGAUGAUGCUUAGCU |
| | AG |
| CLN11976 | GGGAGACAAGAAUAAAGCGAGUUUCAGCACACACAAA |
| SEQ ID NO 265 | CUAGCCAACUAAUCACAAGAGUCGAUGAUGCUUAGCU |
| | AG |
| CLN11977 | GGGAGACAAGAAUAAAGCGAGUUGUAUUCUUGCAACC |
| SEQ ID NO 266 | UAGGUAACUUCUCACAAAGAGUCGAUGAUGCUUAGCU |
| | AG |
| CLN11978 | GGGAGACAAGAAUAAAGCGAGUUAGCUUAGUCAAACU |
| SEQ ID NO 267 | AACUAACCUCAACACUAAGAGUCGAUGAUGCUUAGCU |
| | AG |
| CLN11962 | GGGAGACAAGAAUAAAGCGAGUUUCCACAUCCAGCAA |
| SEQ ID NO 268 | CUAACUAACUUAUCACAAGAGUCGAUGAUGCUUAGCU |
| | AG |
| CLN11974 | GGGAGACAAGAAUAAAGCGAGUUAUAUUCUGUCACAC |
| SEQ ID NO 269 | UAGCUAACUUAUCACAAAGAGUCGAUGAUGCUUAGCU |
| | AG |
| CLN11975 | GGGAGACAAGAAUAAAGCGAGUUUCCUCACACCAAAC |
| SEQ ID NO 270 | UAUCUUCCAACAUCACAAGAGUCGAUGAUGCUUAGCU |
| | AG |
| CLN10242 | GGGAGACAAGAAUAAAGCGAGUUACGCUAUGUCAAAC |
| SEQ ID NO 271 | UAGCUAACUACAACACAAGAGUCGAUGAUGCUUAGCU |
| | AG |
| CLN11973 | GGGAGACAAGAAUAAAGCGAGUUAUAUUCUUGCAUAC |
| SEQ ID NO 272 | UAUCUAACAUCAACACAAGAGUCGAUGAUGCUUAGCU |
| | AG |
| CLN11971 | GGGAGACAAGAAUAAAGCGAGUUUCCACGCAAACAAA |
| SEQ ID NO 273 | ACUAGUUAGCACUCACAAGAGUCGAUGAUGCUUAGCU |
| | AG |
| CLN11972 | GGGAGACAAGAAUAAAGCGAGUUUCUUCACAUAACAA |
| SEQ ID NO 274 | CUAGCUCUCAAAUCACAAGAGUCGAUGAUGCUUAGCU |
| | AG |
| CLN11968 | GGGAGACAAGAAUAAAGCGAGUUUCUUCACGCAAAAC |
| SEQ ID NO 275 | CUAUCUAACUUAUCACAAGAGUCGAUGAUGCUUAGCU |
| | AG |
| CLN11969 | GGGAGACAAGAAUAAAGCGAGUUACCACAAAUCAACA |
| SEQ ID NO 276 | CUAGUUAACACAUCACAAGAGUCGAUGAUGCUUAGCU |
| | AG |
| CLN11970 | GGGAGACAAGAAUAAAGCGAGUUAGCUUUGUCAAUCU |
| SEQ ID NO 277 | AGUUUAACUUCUUCGCAAGAGUCGAUGAUGCUUAGCU |
| | AG |
| CLN11965 | GGGAGACAAGAAUAAAGCGAGUUUCCACGGUCCACAA |
| SEQ ID NO 278 | CUAUCUAACAUAUCACAAGAGUCGAUGAUGCUUAGCU |
| | AG |
| CLN11966 | GGGAGACAAGAAUAAAGCGAGUUUCCUUGACAACAAA |
| SEQ ID NO 279 | CUAACUAACUUAUCACAAGAGUCGAUGAUGCUUAGCU |
| | AG |
| CLN11967 | GGGAGACAAGAAUAAAGCGAGUUUUCCUCACAACCAC |
| SEQ ID NO 280 | AAAUCUAACUUAUCACAAGAGUCGAUGAUGCUUAGCU |
| | AG |
| CLN11963 | GGGAGACAAGAAUAAAGCGAGUUGUCCUUUAAUUCUU |
| SEQ ID NO 281 | UGGCUAACUAAAUCACAAGAGUCGAUGAUGCUUAGCU |
| | AG |
| CLN11964 | GGGAGACAAGAAUAAAGCGAGUUGUAUUCUUGCACAC |
| SEQ ID NO 282 | UAGCCUCCAUCACACCAAGAGUCGAUGAUGCUUAGCU |
| | AG |
| CLN11959 | GGGAGACAAGAAUAAAGCGAGUUUCCGCAAAACAAAC |
| SEQ ID NO 283 | CUAGCAAACUUAUCACAAGAGUCGAUGAUGCUUAGCU |
| | AG |
| CLN11960 | GGGAGACAAGAAUAAAGCGAGUUUCUUCAAAUACACA |
| SEQ ID NO 284 | CUAGUUAACUAUUCACAAGAGUCGAUGAUGCUUAGCU |
| | AG |
| CLN11961 | GGGAGACAAGAAUAAAGCGAGUUAUACAAUUCUACAU |
| SEQ ID NO 285 | AGCGCGCCUACCAACUAAGAGUCGAUGAUGCUUAGCU |
| | AG |
| CLN11956 | GGGAGACAAGAAUAAAGCGAGUUGUUCUUCCAUCCAA |
| SEQ ID NO 286 | CAGCUCUCGAUAUCACAAGAGUCGAUGAUGCUUAGCU |
| | AG |
| CLN11957 | GGGAGACAAGAAUAAAGCGAGUUUUCCUCAAAUCAUA |
| SEQ ID NO 287 | AUGGCUAACUUAUCACAAGAGUCGAUGAUGCUUAGCU |
| | AG |
| CLN11958 | GGGAGACAAGAAUAAAGCGAGUUUGCUCUCUCAACAC |
| SEQ ID NO 288 | CUAUCUAACUAAUCACAAGAGUCGAUGAUGCUUAGCU |
| | AG |
| CLN11954 | GGGAGACAAGAAUAAAGCGAGUUUCUACGCAACCAAA |
| SEQ ID NO 289 | CUAGUUUACAUUUCACAAGAGUCGAUGAUGCUUAGCU |
| | AG |
| CLN11955 | GGGAGACAAGAAUAAAGCGAGUUGAUCUAAACAUUUC |
| SEQ ID NO 290 | UCAUAACAAAUUGUUCAAGAGUCGAUGAUGCUUAGCU |
| | AG |
| CLN11952 | GGGAGACAAGAAUAAAGCGAGUUUCCACCAAACAAUC |
| SEQ ID NO 291 | UAUCUAACUAAUUCACAAGAGUCGAUGAUGCUUAGCU |
| | AG |
| CLN11953 | GGGAGACAAGAAUAAAGCGAGUUUCCACAAACACAAU |
| SEQ ID NO 292 | CUAACUAGCUCAACACAAGAGUCGAUGAUGCUUAGCU |
| | AG |
| CLN11765 | GGGAGACAAGAAUAAAGCGAGUUGUAUUCUUGCGAUC |
| SEQ ID NO 293 | UAGCUAUCAUAUUCACAAGAGUCGAUGAUGCUUAGCU |
| | AG |
| CLN11950 | GGGAGACAAGAAUAAAGCGAGUUCCCACAAACCAUCA |
| SEQ ID NO 294 | ACAGCUAAUAUUUCACAAGAGUCGAUGAUGCUUAGCU |
| | AG |
| CLN11951 | GGGAGACAAGAAUAAAGCGAGUUGGCCUUUACACAUU |
| SEQ ID NO 295 | AAAGCACUACGUUAUAAAGAGUCGAUGAUGCUUAGCU |
| | AG |
| CLN11949 | GGGAGACAAGAAUAAAGCGAGUUUGUAUUCCUUCAUA |
| SEQ ID NO 296 | CUAACUAACUUAUCACAAGAGUCGAUGAUGCUUAGCU |
| | AG |
| CLN11940 | GGGAGACAAGAAUAAAGCGAGUUUCCACAAAAUCAAU |
| SEQ ID NO 297 | CUAACUAACAUAUCACAAGAGUCGAUGAUGCUUAGCU |
| | AG |
| CLN11947 | GGGAGACAAGAAUAAAGCGAGUUUUCACACACACACA |
| SEQ ID NO 298 | CUAACUUUCACAUCACAAGAGUCGAUGAUGCUUAGCU |
| | AG |
| CLN11948 | GGGAGACAAGAAUAAAGCGAGUUUCCACACACCUCAA |
| SEQ ID NO 299 | CUAACUAACAGAUCACAAGAGUCGAUGAUGCUUAGCU |
| | AG |
| CLN11944 | GGGAGACAAGAAUAAAGCGAGUUGUAUUACUGUCAUA |
| SEQ ID NO 300 | CUAGUUAACUUAUCACAAGAGUCGAUGAUGCUUAGCU |
| | AG |
| CLN11945 | GGGAGACAAGAAUAAAGCGAGUUUCCACCAAUCCAAC |
| SEQ ID NO 301 | UAUCUCACGCCAUCACAAGAGUCGAUGAUGCUUAGCU |
| | AG |
| CLN11946 | GGGAGACAAGAAUAAAGCGAGUUUUCACAUCAACCAU |
| SEQ ID NO 302 | CUAGCUUUCAAAUCACAAGAGUCGAUGAUGCUUAGCU |
| | AG |
| CLN11942 | GGGAGACAAGAAUAAAGCGAGUUUCCACAAAUCCAAU |
| SEQ ID NO 303 | CUAGCAAACCCAUCACAAGAGUCGAUGAUGCUUAGCU |
| | AG |
| CLN11943 | GGGAGACAAGAAUAAAGCGAGUUUCUUCAACCAACAA |
| SEQ ID NO 304 | UUAGCUCUCUUAUCACAAGAGUCGAUGAUGCUUAGCU |
| | AG |
| CLN11938 | GGGAGACAAGAAUAAAGCGAGUUUCAUCAUGCAUAAU |
| SEQ ID NO 305 | CUAGCUAACUUAUCACAAGAGUCGAUGAUGCUUAGCU |
| | AG |
| CLN11939 | GGGAGACAAGAAUAAAGCGAGUUGUUCUUCCAUCAAC |
| SEQ ID NO 306 | AAGUUAACUACAUCACAAGAGUCGAUGAUGCUUAGCU |
| | AG |
| CLN11770 | GGGAGACAAGAAUAAAGCGAGUUUGUAUUUUCGAACU |
| SEQ ID NO 307 | UUCUAACACAUCACAAGAGUCGAUGAUGCUUAGCUAG |
| CLN10268 | GGGAGACAAGAAUAAAGCGAGUUAAACUAGUGCUAAC |
| SEQ ID NO 308 | UCAACACACUUAAUAAAGAGUCGAUGAUGCUUAGCUA |
| | G |
| CLN11769 | GGGAGACAAGAAUAAAGCGAGUUUCCACCACAAUCCAC |
| SEQ ID NO 309 | CUAGUUUUCAUAUCACAAGAGUCGAUGAUGCUUAGCUA |
| | G |
| CLN10238 | GGGAGACAAGAAUAAAGCGAGUUCUUCCUAAACUGCCG |
| SEQ ID NO 310 | AAACUGCACAAUAUCAAGAGUCGAUGAUGCUUAGCUAG |
| CLN10236 | GGGAGACAAGAAUAAAGCGAGUUUGCUAUGUCAAUCUA |
| SEQID NO 311 | GCUUCCAUCAGCACUAAGAGUCGAUGAUGCUUAGCUAG |
| CLN11768 | GGGAGACAAGAAUAAAGCGAGUUUUGCCGUCAAUAUGC |
| SEQ ID NO 312 | AUGUUAUCGUCUGUAAAGAGUCGAUGAUGCUUAGCUAG |
| CLN11767 | GGGAGACAAGAAUAAAGCGAGUUCCGUAGUGCGAGGCU |
| SEQ ID NO 313 | CAAGGUCUACCACUGAAGAGUCGAUGAUGCUUAGCUAG |
| CLN11766 | GGGAGACAAGAAUAAAGCGAGUUACAAACAAUGGCGUG |
| SEQ ID NO 314 | CUCAUGAUCGUGUUAAAGAGUCGAUGAUGCUUAGCUAG |
| CLN11764 | GGGAGACAAGAAUAAAGCGAGUUCUAUUUGCAAACACC |
| SEQ ID NO 315 | AAGACCCUCUACGUGAAGAGUCGAUGAUGCUUAGCUAG |
| CLN11762 | GGGAGACAAGAAUAAAGCGAGUUGAUCUGAUUUCAAGU |
| SEQ ID NO 316 | UAUCAUUGCAUAAUCAAGAGUCGAUGAUGCUUAGCUAG |
| CLN11763 | GGGAGACAAGAAUAAAGCGAGUUACCCUGUUUUUACCU |
| SEQ ID NO 317 | AACUAGCAUAUCACUAAGAGUCGAUGAUGCUUAGCUAG |
| CLN11761 | GGGAGACAAGAAUAAAGCGAGUUUCCACAAAUGCCUCC |
| SEQ ID NO 318 | UAGCUAACUUAUCACAAGAGUCGAUGAUGCUUAGCUAG |
| CLN11758 | GGGAGACAAGAAUAAAGCGAGUUUCGGCACAAUCCAUC |
| SEQ ID NO 319 | UAGUUAACAUAUCACAAGAGUCGAUGAUGCUUAGCUAG |
| CLN11759 | GGGAGACAAGAAUAAAGCGAGUUUCACGCAUUGAUCAC |
| SEQ ID NO 320 | AGUACAUUAGGAUUCAAGAGUCGAUGAUGCUUAGCUAG |
| CLN11760 | GGGAGACAAGAAUAAAGCGAGUUGGCCUUACAUGUACU |
| SEQ ID NO 321 | AUAAUUCAUAUGUCAAGAGUCGAUGAUGCUUAGCUAG |
| CLN11756 | GGGAGACAAGAAUAAAGCGAGUUUCCACAAGCCAAACC |
| SEQ ID NO 322 | UAACUAACAUAUCACAAGAGUCGAUGAUGCUUAGCUAG |
| CLN11757 | GGGAGACAAGAAUAAAGCGAGUUUCACCAACCACACCC |
| SEQ ID NO 323 | UAGUUUUCAUAUCACAAGAGUCGAUGAUGCUUAGCUAG |
| CLN11755 | GGGAGACAAGAAUAAAGCGAGUUCAUGCCUUAUAUUAC |
| SEQ ID NO 324 | UUGCAGAGUAGUAUCAAGAGUCGAUGAUGCUUAGCUAG |
| CLN11753 | GGGAGACAAGAAUAAAGCGAGUUUGCUAUGUCAUUCCA |
| SEQ ID NO 325 | GUUCACAUCUUCACAAAGAGUCGAUGAUGCUUAGCUAG |
| CLN11754 | GGGAGACAAGAAUAAAGCGAGUUUCAUUCUGGUCGAAA |
| SEQ ID NO 326 | CAGUUAACAUGUCACAAGAGUCGAUGAUGCUUAGCUAG |
| CLN10218 | GGGAGACAAGAAUAAAGCGAGUUGGCCUUACAUUGUAC |
| SEQ ID NO 327 | UAUAAUUCAUAUGUCAAGAGUCGAUGAUGCUUAGCUAG |
| CLN11742 | GGGAGACAAGAAUAAAGCGAGUUGUAUAUCUUAACACA |
| SEQ ID NO 328 | AGCGAGGUAAUUUGCAAGAGUCGAUGAUGCUUAGCUAG |

What is claimed is:

1. A method for selecting oligonucleotides, preferably aptamers, that persist in a biological compartment comprising: a) preparing a candidate mixture of oligonucleotides; b) introducing said candidate mixture of oligonucleotides into a biological compartment; c) partitioning the oligonucleotides having an increased persistence in the biological compartment from the remainder of the candidate mixture and d) amplifying the oligonucleotides having an increased persistence in the biological compartment to yield a mixture of oligonucleotides, preferably aptamers, enriched for oligonucleotides, preferably aptamers, with relatively greater persistence in a biological compartment than the remainder of the candidate mixture.

2. The method of claim 1 further comprising step: e) repeating steps b), c) and d).

3. The method of claim 1 wherein said candidate mixture is comprised of single-stranded oligonucleotides.

4. The method of claim 3 wherein said single-stranded oligonucleotides are nuclease stabilized.

5. The method of claim 3 wherein said nuclease stabilized single-stranded oligonucleotides are MNA.

6. The method of claim 1 wherein said biological compartment is within a living organism.

7. The method of claim 6 wherein said biological compartment comprises at least one tissue.

8. The method of claim 7 wherein said tissue is blood.

9. The methods of claim 8 wherein said blood is confined within the circulatory system.

10. The method of claim 1 wherein said persistence is in the range of 5 mins. to 64 hrs.

11. A method for selecting oligonucleotides, preferably aptamers, that persist in a specific organ comprising the steps of: a) preparing a candidate mixture of oligonucleotides, b) introducing said candidate mixture of oligonucleotides into an artery that perfuses an organ, c) waiting for a period of time to elapse, d) partitioning the oligonucleotides having an increased persistence in said organ from the remainder of the candidate mixture and e) amplifying the oligonucleotides having an increased persistence in said organ to yield a mixture of an oligonucleotides, preferably aptamers, enriched for oligonucleotides, preferably an aptamers, with relatively greater persistence in said organ than the remainder of the candidate mixture.

12. The method of claim 11 further comprising step: f) repeating steps b), c), d) and e).

13. The method of claim 11 wherein said candidate mixture is comprised of single-stranded oligonucleotides.

14. The method of claim 13 wherein said single-stranded oligonucleotides are nuclease stabilized.

15. The method of claim 13 wherein said nuclease stabilized single-stranded oligonucleotides are MNA.

16. The method of claim 11 wherein said organ is selected from the group heart, lung, brain, eye, stomach, spleen, bone, pancreas, kidney, liver, intestine, skin, urinary bladder, ovary, uterus and testicle.

17. The method of claim 11 wherein said persistence is in the range of 5 mins. to 64 hrs.

18. A method for selecting oligonucleotides, preferably aptamers, that persist in a tumor comprising the steps of: a) preparing a candidate mixture of oligonucleotides, b) introducing said candidate mixture of oligonucleotides into an artery that perfuses a tumor, c) waiting for a period of time to elapse, d) partitioning the oligonucleotides having an increased persistence in said tumor from the remainder of the candidate mixture and e) amplifying the oligonucleotides having an increased persistence in said tumor to yield a mixture of an oligonucleotides, preferably aptamers, enriched for oligonucleotides, preferably an aptamers, with relatively greater persistence in said tumor than the remainder of the candidate mixture.

19. The method of claim 18 further comprising step: f) repeating steps b), c), d) and e).
